SEQUENCE LISTING (346 sequences)

SEQ ID NO: 1 - DNA, 19 bp, synthetic construct
  source 1..19: mol_type = other DNA
gactcaaagtgggttcggg

SEQ ID NO: 2 - DNA, 25 bp, synthetic construct
  source 1..25: mol_type = other DNA
gcatcaggaggagctttttgtctta

SEQ ID NO: 3 - DNA, 28 bp, synthetic construct
  source 1..28: mol_type = other DNA
aaaatgactggatataagcacttggttg

SEQ ID NO: 4 - DNA, 21 bp, synthetic construct
  source 1..21: mol_type = other DNA
gctgcgtatcattttcatcgg

SEQ ID NO: 5 - DNA, 20 bp, synthetic construct
  source 1..20: mol_type = other DNA
agcgaacgcgttttacaaaa

SEQ ID NO: 6 - DNA, 25 bp, synthetic construct
  source 1..25: mol_type = other DNA
gctttaaaccatggtttatcccagg

SEQ ID NO: 7 - DNA, 17 bp, synthetic construct
  source 1..17: mol_type = other DNA
cgcagcacagctgaacc

SEQ ID NO: 8 - DNA, 27 bp, synthetic construct
  source 1..27: mol_type = other DNA
ccagtggtcatattaacgtatccagag

SEQ ID NO: 9 - DNA, 28 bp, synthetic construct
  source 1..28: mol_type = other DNA
aaactgtatatcttcctcctcaatagca

SEQ ID NO: 10 - DNA, 23 bp, synthetic construct
  source 1..23: mol_type = other DNA
ccagtcactacaccaagcttctt

SEQ ID NO: 11 - DNA, 20 bp, synthetic construct
  source 1..20: mol_type = other DNA
cgtaactgggttgtccgtaa

SEQ ID NO: 12 - DNA, 23 bp, synthetic construct
  source 1..23: mol_type = other DNA
atcagcagtgggtagaagtcact

SEQ ID NO: 13 - DNA, 19 bp, synthetic construct
  source 1..19: mol_type = other DNA
gcagaccggcaacaaacag

SEQ ID NO: 14 - DNA, 18 bp, synthetic construct
  source 1..18: mol_type = other DNA
cggatgagcaggagcact

SEQ ID NO: 15 - DNA, 24 bp, synthetic construct
  source 1..24: mol_type = other DNA
atgcgtataggtgtttctttgtcg

SEQ ID NO: 16 - DNA, 16 bp, synthetic construct
  source 1..16: mol_type = other DNA
atggcaactggcgggc

SEQ ID NO: 17 - DNA, 28 bp, synthetic construct
  source 1..28: mol_type = other DNA
tgacattgttcttcttcattagattcag

SEQ ID NO: 18 - DNA, 28 bp, synthetic construct
  source 1..28: mol_type = other DNA
atattccattaagcatcataattgggat

SEQ ID NO: 19 - DNA, 19 bp, synthetic construct
  source 1..19: mol_type = other DNA
ggacatggtttgggtggag

SEQ ID NO: 20 - DNA, 23 bp, synthetic construct
  source 1..23: mol_type = other DNA
catacatgaaccatctcccagag

SEQ ID NO: 21 - DNA, 20 bp, synthetic construct
  source 1..20: mol_type = other DNA
tacatgcgtccctcaacaca

SEQ ID NO: 22 - DNA, 23 bp, synthetic construct
  source 1..23: mol_type = other DNA
ctaggtagtggtttgggtggaga

SEQ ID NO: 23 - DNA, 27 bp, synthetic construct
  source 1..27: mol_type = other DNA
gtgaataagcgaaatattttgggtaga

SEQ ID NO: 24 - DNA, 25 bp, synthetic construct
  source 1..25: mol_type = other DNA
cctaaaccatgtccttcagctgaat

SEQ ID NO: 25 - DNA, 25 bp, synthetic construct
  source 1..25: mol_type = other DNA
acagttgtttcctgctcgttgaata

SEQ ID NO: 26 - DNA, 24 bp, synthetic construct
  source 1..24: mol_type = other DNA
gttctctctccagaacttatgtcg

SEQ ID NO: 27 - DNA, 23 bp, synthetic construct
  source 1..23: mol_type = other DNA
tgttgccacttacaattcaacaa

SEQ ID NO: 28 - DNA, 27 bp, synthetic construct
  source 1..27: mol_type = other DNA
gaaaatgtatccatacatcgaacgaca

SEQ ID NO: 29 - DNA, 23 bp, synthetic construct
  source 1..23: mol_type = other DNA
aggtaaaaacgctcatgatgcta

SEQ ID NO: 30 - DNA, 28 bp, synthetic construct
  source 1..28: mol_type = other DNA
ttagaataaatggatggatagtcctgta

SEQ ID NO: 31 - DNA, 27 bp, synthetic construct
  source 1..27: mol_type = other DNA
ttatgggcaatagcacattcaaaggta

SEQ ID NO: 32 - DNA, 25 bp, synthetic construct
  source 1..25: mol_type = other DNA
gaggatgatttgcccttcactcatt

SEQ ID NO: 33 - DNA, 20 bp, synthetic construct
  source 1..20: mol_type = other DNA
tgagatcccgtcgtatgagt

SEQ ID NO: 34 - DNA, 22 bp, synthetic construct
  source 1..22: mol_type = other DNA
tttcccttcaagacctttgccg

SEQ ID NO: 35 - DNA, 28 bp, synthetic construct
  source 1..28: mol_type = other DNA
gcagaaaaatatcttaccatcttttcca

SEQ ID NO: 36 - DNA, 20 bp, synthetic construct
  source 1..20: mol_type = other DNA
aactactccgtgaaacgcat

SEQ ID NO: 37 - DNA, 18 bp, synthetic construct
  source 1..18: mol_type = other DNA
ttgctcaaaacgcggcca

SEQ ID NO: 38 - DNA, 28 bp, synthetic construct
  source 1..28: mol_type = other DNA
agatttccaggatactatagcaagattt

SEQ ID NO: 39 - DNA, 28 bp, synthetic construct
  source 1..28: mol_type = other DNA
ctttagaacgtttttgtggtgtttcatg

SEQ ID NO: 40 - DNA, 26 bp, synthetic construct
  source 1..26: mol_type = other DNA
cataggcatgctagttcaaatagggt

SEQ ID NO: 41 - DNA, 18 bp, synthetic construct
  source 1..18: mol_type = other DNA
ccgctctctctgcagacc

SEQ ID NO: 42 - DNA, 25 bp, synthetic construct
  source 1..25: mol_type = other DNA
gattttatttgtattcggccacgat

SEQ ID NO: 43 - DNA, 27 bp, synthetic construct
  source 1..27: mol_type = other DNA
tttggatatttacagctcagatgcttc

SEQ ID NO: 44 - DNA, 28 bp, synthetic construct
  source 1..28: mol_type = other DNA
agaatattcccaaatgatggataagttt

SEQ ID NO: 45 - DNA, 24 bp, synthetic construct
  source 1..24: mol_type = other DNA
ccaaagaaactctagaggacaacg

SEQ ID NO: 46 - DNA, 28 bp, synthetic construct
  source 1..28: mol_type = other DNA
gccccttaattgtataacaaatttgttt

SEQ ID NO: 47 - DNA, 23 bp, synthetic construct
  source 1..23: mol_type = other DNA
gacatatcatgtcatggacgtct

SEQ ID NO: 48 - DNA, 22 bp, synthetic construct
  source 1..22: mol_type = other DNA
gtaaggacgccatttgttttct

SEQ ID NO: 49 - DNA, 25 bp, synthetic construct
  source 1..25: mol_type = other DNA
agtgactatgattatacgttgcagc

SEQ ID NO: 50 - DNA, 20 bp, synthetic construct
  source 1..20: mol_type = other DNA
ctttactagcgccgcttcga

SEQ ID NO: 51 - DNA, 26 bp, synthetic construct
  source 1..26: mol_type = other DNA
gtttccaagagtggattatgacaaaa

SEQ ID NO: 52 - DNA, 22 bp, synthetic construct
  source 1..22: mol_type = other DNA
cttaacgcctgtatccgaaccc

SEQ ID NO: 53 - DNA, 28 bp, synthetic construct
  source 1..28: mol_type = other DNA
ccctggcattacgacataattttacaaa

SEQ ID NO: 54 - DNA, 26 bp, synthetic construct
  source 1..26: mol_type = other DNA
tgcaaatctctttttcacgacacaac

SEQ ID NO: 55 - DNA, 18 bp, synthetic construct
  source 1..18: mol_type = other DNA
ttgagccacgcgaacagt

SEQ ID NO: 56 - DNA, 28 bp, synthetic construct
  source 1..28: mol_type = other DNA
tatcgtaacgttctataaagttttcgaa

SEQ ID NO: 57 - DNA, 26 bp, synthetic construct
  source 1..26: mol_type = other DNA
aaaaacaacgattaaggaggctatgc

SEQ ID NO: 58 - DNA, 19 bp, synthetic construct
  source 1..19: mol_type = other DNA
cgctacgcgtgaagaacat

SEQ ID NO: 59 - DNA, 28 bp, synthetic construct
  source 1..28: mol_type = other DNA
cgagaaaagtttttaacaccagaaattc

SEQ ID NO: 60 - DNA, 28 bp, synthetic construct
  source 1..28: mol_type = other DNA
atcatctctttgaccattttcttctgtt

SEQ ID NO: 61 - DNA, 20 bp, synthetic construct
  source 1..20: mol_type = other DNA
cgtctctagacgacttctgc

SEQ ID NO: 62 - DNA, 24 bp, synthetic construct
  source 1..24: mol_type = other DNA
attcagaaagatcaaaaggtgcag

SEQ ID NO: 63 - DNA, 16 bp, synthetic construct
  source 1..16: mol_type = other DNA
tgaaagcccgcggcac

SEQ ID NO: 64 - DNA, 23 bp, synthetic construct
  source 1..23: mol_type = other DNA
cttctttggggagtgtactcagc

SEQ ID NO: 65 - DNA, 21 bp, synthetic construct
  source 1..21: mol_type = other DNA
caaggtttaccactccctgct

SEQ ID NO: 66 - DNA, 16 bp, synthetic construct
  source 1..16: mol_type = other DNA
ttgtcgtccggcgtcc

SEQ ID NO: 67 - DNA, 19 bp, synthetic construct
  source 1..19: mol_type = other DNA
aacaacggcagacagcaaa

SEQ ID NO: 68 - DNA, 23 bp, synthetic construct
  source 1..23: mol_type = other DNA
ggcttctccgggtttctattctt

SEQ ID NO: 69 - DNA, 23 bp, synthetic construct
  source 1..23: mol_type = other DNA
ccatttccctctagcgactgaag

SEQ ID NO: 70 - DNA, 24 bp, synthetic construct
  source 1..24: mol_type = other DNA
tgtaacttattctccctgagtctg

SEQ ID NO: 71 - DNA, 16 bp, synthetic construct
  source 1..16: mol_type = other DNA
agtcggccgcgtgaca

SEQ ID NO: 72 - DNA, 23 bp, synthetic construct
  source 1..23: mol_type = other DNA
aagcctagatgatttctgcttcg

SEQ ID NO: 73 - DNA, 26 bp, synthetic construct
  source 1..26: mol_type = other DNA
ctgtggggtatttatagcttcacaga

SEQ ID NO: 74 - DNA, 19 bp, synthetic construct
  source 1..19: mol_type = other DNA
ggagacctgcagcactttc

SEQ ID NO: 75 - DNA, 21 bp, synthetic construct
  source 1..21: mol_type = other DNA
ggtaaccgagcatacgctgtg

SEQ ID NO: 76 - DNA, 21 bp, synthetic construct
  source 1..21: mol_type = other DNA
ggccatacttgacgaggttaa

SEQ ID NO: 77 - DNA, 23 bp, synthetic construct
  source 1..23: mol_type = other DNA
tcaggctttttctttttggcctg

SEQ ID NO: 78 - DNA, 22 bp, synthetic construct
  source 1..22: mol_type = other DNA
cggtaagaaccagagccagaag

SEQ ID NO: 79 - DNA, 18 bp, synthetic construct
  source 1..18: mol_type = other DNA
accttcccgctggatgaa

SEQ ID NO: 80 - DNA, 21 bp, synthetic construct
  source 1..21: mol_type = other DNA
ctgaagatgacatccggcacc

SEQ ID NO: 81 - DNA, 18 bp, synthetic construct
  source 1..18: mol_type = other DNA
ccgtccacgtagatcggg

SEQ ID NO: 82 - DNA, 19 bp, synthetic construct
  source 1..19: mol_type = other DNA
ccacctgggactacacgct

SEQ ID NO: 83 - DNA, 16 bp, synthetic construct
  source 1..16: mol_type = other DNA
gagggccgagccgaag

SEQ ID NO: 84 - DNA, 16 bp, synthetic construct
  source 1..16: mol_type = other DNA
tctccgccgagacgac

SEQ ID NO: 85 - DNA, 18 bp, synthetic construct
  source 1..18: mol_type = other DNA
cagacgtcgaagccggag

SEQ ID NO: 86 - DNA, 16 bp, synthetic construct
  source 1..16: mol_type = other DNA
gggtcgtacgcgagcc

SEQ ID NO: 87 - DNA, 16 bp, synthetic construct
  source 1..16: mol_type = other DNA
ggcgagaagagctgcg

SEQ ID NO: 88 - DNA, 19 bp, synthetic construct
  source 1..19: mol_type = other DNA
cctctacacgctgcacgac

SEQ ID NO: 89 - DNA, 16 bp, synthetic construct
  source 1..16: mol_type = other DNA
tggcggtctttggcgc

SEQ ID NO: 90 - DNA, 16 bp, synthetic construct
  source 1..16: mol_type = other DNA
cgagcgccagcagcag

SEQ ID NO: 91 - DNA, 18 bp, synthetic construct
  source 1..18: mol_type = other DNA
acctcctcgacgatgcag

SEQ ID NO: 92 - DNA, 16 bp, synthetic construct
  source 1..16: mol_type = other DNA
cctgaacgcgctcggc

SEQ ID NO: 93 - DNA, 21 bp, synthetic construct
  source 1..21: mol_type = other DNA
ctgcagttcacctacgaccac

SEQ ID NO: 94 - DNA, 16 bp, synthetic construct
  source 1..16: mol_type = other DNA
caggcgcgacatctcg

SEQ ID NO: 95 - DNA, 17 bp, synthetic construct
  source 1..17: mol_type = other DNA
ccgcaaccccatgaagg

SEQ ID NO: 96 - DNA, 17 bp, synthetic construct
  source 1..17: mol_type = other DNA
ccgcaaccccatgaagg

SEQ ID NO: 97 - DNA, 20 bp, synthetic construct
  source 1..20: mol_type = other DNA
tccaaggaggcgttaccgaa

SEQ ID NO: 98 - DNA, 18 bp, synthetic construct
  source 1..18: mol_type = other DNA
gggagaggcgggtgttga

SEQ ID NO: 99 - DNA, 24 bp, synthetic construct
  source 1..24: mol_type = other DNA
tcaatagtggaatctaggacaggt

SEQ ID NO: 100 - DNA, 28 bp, synthetic construct
  source 1..28: mol_type = other DNA
tagatgataactttgtaacaaaggccac

SEQ ID NO: 101 - DNA, 23 bp, synthetic construct
  source 1..23: mol_type = other DNA
tcctaccactcccggtactttac

SEQ ID NO: 102 - DNA, 22 bp, synthetic construct
  source 1..22: mol_type = other DNA
tggtctacatttccagcagttt

SEQ ID NO: 103 - DNA, 21 bp, synthetic construct
  source 1..21: mol_type = other DNA
gcaagaagagtggaagaagcg

SEQ ID NO: 104 - DNA, 19 bp, synthetic construct
  source 1..19: mol_type = other DNA
ctcctcattgccttcctcg

SEQ ID NO: 105 - DNA, 25 bp, synthetic construct
  source 1..25: mol_type = other DNA
gcagtattctgattaccagcaatca

SEQ ID NO: 106 - DNA, 24 bp, synthetic construct
  source 1..24: mol_type = other DNA
gtggattgttctgtagcattcttc

SEQ ID NO: 107 - DNA, 28 bp, synthetic construct
  source 1..28: mol_type = other DNA
cttactgatagaatgtggagctcctaga

SEQ ID NO: 108 - DNA, 21 bp, synthetic construct
  source 1..21: mol_type = other DNA
gcggaaatttctgacaaacgt

SEQ ID NO: 109 - DNA, 25 bp, synthetic construct
  source 1..25: mol_type = other DNA
gccagccataaaagtcatcaataac

SEQ ID NO: 110 - DNA, 27 bp, synthetic construct
  source 1..27: mol_type = other DNA
gaaaaagcaaatgggctgctaattttg

SEQ ID NO: 111 - DNA, 28 bp, synthetic construct
  source 1..28: mol_type = other DNA
tggtggagtatttctttgtgtagtatgt

SEQ ID NO: 112 - DNA, 26 bp, synthetic construct
  source 1..26: mol_type = other DNA
atgagacacagagctatattcagaag

SEQ ID NO: 113 - DNA, 28 bp, synthetic construct
  source 1..28: mol_type = other DNA
gatactaaagatgaaagttacactcagc

SEQ ID NO: 114 - DNA, 19 bp, synthetic construct
  source 1..19: mol_type = other DNA
ggagccaagtgtttgtggt

SEQ ID NO: 115 - DNA, 26 bp, synthetic construct
  source 1..26: mol_type = other DNA
ccgtagaagtctgtcattccagtttt

SEQ ID NO: 116 - DNA, 19 bp, synthetic construct
  source 1..19: mol_type = other DNA
ggaactaccagcgctcacc

SEQ ID NO: 117 - DNA, 19 bp, synthetic construct
  source 1..19: mol_type = other DNA
gagggaaagcccgaaacac

SEQ ID NO: 118 - DNA, 24 bp, synthetic construct
  source 1..24: mol_type = other DNA
ttccaacctctttgccgataataa

SEQ ID NO: 119 - DNA, 27 bp, synthetic construct
  source 1..27: mol_type = other DNA
ggagtggtattcatcggagaatattcg

SEQ ID NO: 120 - DNA, 23 bp, synthetic construct
  source 1..23: mol_type = other DNA
tgtctccatgtctttctttaccc

SEQ ID NO: 121 - DNA, 17 bp, synthetic construct
  source 1..17: mol_type = other DNA
cccgacgtctccgtcag

SEQ ID NO: 122 - DNA, 20 bp, synthetic construct
  source 1..20: mol_type = other DNA
aggccagcgagtattgcaag

SEQ ID NO: 123 - DNA, 24 bp, synthetic construct
  source 1..24: mol_type = other DNA
gtaaaccgcgtgattttaaaactg

SEQ ID NO: 124 - DNA, 24 bp, synthetic construct
  source 1..24: mol_type = other DNA
ccccattataaccatcccaccaag

SEQ ID NO: 125 - DNA, 17 bp, synthetic construct
  source 1..17: mol_type = other DNA
gcaagtcacagcgcact

SEQ ID NO: 126 - DNA, 26 bp, synthetic construct
  source 1..26: mol_type = other DNA
aactatacgtgctgtaagttacagag

SEQ ID NO: 127 - DNA, 28 bp, synthetic construct
  source 1..28: mol_type = other DNA
ttgatgatattgagttttgctcccatac

SEQ ID NO: 128 - DNA, 19 bp, synthetic construct
  source 1..19: mol_type = other DNA
atggtacccctctcaccac

SEQ ID NO: 129 - DNA, 26 bp, synthetic construct
  source 1..26: mol_type = other DNA
ggaccaacttcatctcaagacttgta

SEQ ID NO: 130 - DNA, 24 bp, synthetic construct
  source 1..24: mol_type = other DNA
cctcactttgtgtttgatgatcag

SEQ ID NO: 131 - DNA, 24 bp, synthetic construct
  source 1..24: mol_type = other DNA
tggattttgtgacatgatctccat

SEQ ID NO: 132 - DNA, 23 bp, synthetic construct
  source 1..23: mol_type = other DNA
agcgttgtcatattgagtaccgc

SEQ ID NO: 133 - DNA, 23 bp, synthetic construct
  source 1..23: mol_type = other DNA
agtgcttgatcggtaacacaact

SEQ ID NO: 134 - DNA, 23 bp, synthetic construct
  source 1..23: mol_type = other DNA
acaggaagttttccttacaggtc

SEQ ID NO: 135 - DNA, 28 bp, synthetic construct
  source 1..28: mol_type = other DNA
gttctgttagaactatgtaggtcactat

SEQ ID NO: 136 - DNA, 23 bp, synthetic construct
  source 1..23: mol_type = other DNA
ggcgagtatcagtactggtttga

SEQ ID NO: 137 - DNA, 22 bp, synthetic construct
  source 1..22: mol_type = other DNA
gaagtgagtgaagcctccttat

SEQ ID NO: 138 - DNA, 22 bp, synthetic construct
  source 1..22: mol_type = other DNA
caactgaatgacgggaccgtta

SEQ ID NO: 139 - DNA, 18 bp, synthetic construct
  source 1..18: mol_type = other DNA
tgtggtggtcgcacaatc

SEQ ID NO: 140 - DNA, 21 bp, synthetic construct
  source 1..21: mol_type = other DNA
ggtggacgggtgtcatagagt

SEQ ID NO: 141 - DNA, 19 bp, synthetic construct
  source 1..19: mol_type = other DNA
cccagtggccaattcctca

SEQ ID NO: 142 - DNA, 24 bp, synthetic construct
  source 1..24: mol_type = other DNA
ttcttctctgacgtcaggtcaaac

SEQ ID NO: 143 - DNA, 20 bp, synthetic construct
  source 1..20: mol_type = other DNA
ttcaaagcgacaggcttctt

SEQ ID NO: 144 - DNA, 17 bp, synthetic construct
  source 1..17: mol_type = other DNA
ggagggaccttacgccg

SEQ ID NO: 145 - DNA, 26 bp, synthetic construct
  source 1..26: mol_type = other DNA
acacttccacatggattatggaactg

SEQ ID NO: 146 - DNA, 18 bp, synthetic construct
  source 1..18: mol_type = other DNA
cgtcaggtccagagtgga

SEQ ID NO: 147 - DNA, 19 bp, synthetic construct
  source 1..19: mol_type = other DNA
ggttgtgtctgtggatccc

SEQ ID NO: 148 - DNA, 20 bp, synthetic construct
  source 1..20: mol_type = other DNA
ctgggtcacagaaccaatca

SEQ ID NO: 149 - DNA, 16 bp, synthetic construct
  source 1..16: mol_type = other DNA
gaaaggcgccggcctc

SEQ ID NO: 150 - DNA, 18 bp, synthetic construct
  source 1..18: mol_type = other DNA
cccgaggtaacacgcgac

SEQ ID NO: 151 - DNA, 22 bp, synthetic construct
  source 1..22: mol_type = other DNA
ggaaccgatggactttcgttca

SEQ ID NO: 152 - DNA, 19 bp, synthetic construct
  source 1..19: mol_type = other DNA
cgaccgaataggcggagtg

SEQ ID NO: 153 - DNA, 23 bp, synthetic construct
  source 1..23: mol_type = other DNA
ttcttgtccgaagttagagggct

SEQ ID NO: 154 - DNA, 21 bp, synthetic construct
  source 1..21: mol_type = other DNA
ccatttctcatgaaaacgggc

SEQ ID NO: 155 - DNA, 17 bp, synthetic construct
  source 1..17: mol_type = other DNA
accatgctccgctacga

SEQ ID NO: 156 - DNA, 23 bp, synthetic construct
  source 1..23: mol_type = other DNA
aaaacgtgcaacttgaaatcccg

SEQ ID NO: 157 - DNA, 22 bp, synthetic construct
  source 1..22: mol_type = other DNA
ggattggtcttgtctttagcca

SEQ ID NO: 158 - DNA, 23 bp, synthetic construct
  source 1..23: mol_type = other DNA
gtcttctaaccgaggtcgaaacg

SEQ ID NO: 159 - DNA, 20 bp, synthetic construct
  source 1..20: mol_type = other DNA
gactgcagcgtagacgcttt

SEQ ID NO: 160 - DNA, 19 bp, synthetic construct
  source 1..19: mol_type = other DNA
cagcagagtaactgagcgc

SEQ ID NO: 161 - DNA, 21 bp, synthetic construct
  source 1..21: mol_type = other DNA
gatccagccatttgttccatg

SEQ ID NO: 162 - DNA, 21 bp, synthetic construct
  source 1..21: mol_type = other DNA
cctagtatgcgcaacctgtga

SEQ ID NO: 163 - DNA, 23 bp, synthetic construct
  source 1..23: mol_type = other DNA
cattcgtttctgataggcctgta

SEQ ID NO: 164 - DNA, 23 bp, synthetic construct
  source 1..23: mol_type = other DNA
agcagcagaggctatggaaattg

SEQ ID NO: 165 - DNA, 28 bp, synthetic construct
  source 1..28: mol_type = other DNA
actgttacattcttttctagtactgtgt

SEQ ID NO: 166 - DNA, 28 bp, synthetic construct
  source 1..28: mol_type = other DNA
atgaaggcaatactagtagtcttgctat

SEQ ID NO: 167 - DNA, 26 bp, synthetic construct
  source 1..26: mol_type = other DNA
ataccatccatctatcattcctgtcc

SEQ ID NO: 168 - DNA, 28 bp, synthetic construct
  source 1..28: mol_type = other DNA
tgtccaaaatatgtcaaaagcacaaaat

SEQ ID NO: 169 - DNA, 28 bp, synthetic construct
  source 1..28: mol_type = other DNA
tggtaaatccttgttgattccagcttta

SEQ ID NO: 170 - DNA, 22 bp, synthetic construct
  source 1..22: mol_type = other DNA
ccacaaatgtgatgacacgtgc

SEQ ID NO: 171 - DNA, 25 bp, synthetic construct
  source 1..25: mol_type = other DNA
tctggtagagactttgttggtcagt

SEQ ID NO: 172 - DNA, 25 bp, synthetic construct
  source 1..25: mol_type = other DNA
tctacagaaacttaatatggctggt

SEQ ID NO: 173 - DNA, 28 bp, synthetic construct
  source 1..28: mol_type = other DNA
aaactgtaatcccgaatatcggatctag

SEQ ID NO: 174 - DNA, 25 bp, synthetic construct
  source 1..25: mol_type = other DNA
aattagattccctgtgctgttaatc

SEQ ID NO: 175 - DNA, 26 bp, synthetic construct
  source 1..26: mol_type = other DNA
gtacggtttcaggcatcaaaattctg

SEQ ID NO: 176 - DNA, 24 bp, synthetic construct
  source 1..24: mol_type = other DNA
tgatggaatttctcgttcgttttc

SEQ ID NO: 177 - DNA, 24 bp, synthetic construct
  source 1..24: mol_type = other DNA
attccttattttggaagccatcac

SEQ ID NO: 178 - DNA, 27 bp, synthetic construct
  source 1..27: mol_type = other DNA
ttgaagtaactaatgctactgagctgg

SEQ ID NO: 179 - DNA, 28 bp, synthetic construct
  source 1..28: mol_type = other DNA
ttctacgataaatttcaatgttatcgcc

SEQ ID NO: 180 - DNA, 28 bp, synthetic construct
  source 1..28: mol_type = other DNA
cagataacggtacaacaaatgatgtttc

SEQ ID NO: 181 - DNA, 27 bp, synthetic construct
  source 1..27: mol_type = other DNA
agaacaatctattgtcttaggcacagg

SEQ ID NO: 182 - DNA, 19 bp, synthetic construct
  source 1..19: mol_type = other DNA
ccctgcgcttgggtatttg

SEQ ID NO: 183 - DNA, 22 bp, synthetic construct
  source 1..22: mol_type = other DNA
cttgtcctggatcgcctttatc

SEQ ID NO: 184 - DNA, 17 bp, synthetic construct
  source 1..17: mol_type = other DNA
cgagcagggtcctaggg

SEQ ID NO: 185 - DNA, 26 bp, synthetic construct
  source 1..26: mol_type = other DNA
atacccgctcttgcattaacaatatc

SEQ ID NO: 186 - DNA, 23 bp, synthetic construct
  source 1..23: mol_type = other DNA
aatggtgccagaattaccaacgt

SEQ ID NO: 187 - DNA, 19 bp, synthetic construct
  source 1..19: mol_type = other DNA
agtagcagcaggtgaagct

SEQ ID NO: 188 - DNA, 19 bp, synthetic construct
  source 1..19: mol_type = other DNA
atccgttgctttcgcacca

SEQ ID NO: 189 - DNA, 26 bp, synthetic construct
  source 1..26: mol_type = other DNA
acggatatcgttgttcttgtagtagc

SEQ ID NO: 190 - DNA, 21 bp, synthetic construct
  source 1..21: mol_type = other DNA
ttctacacgctctaggtttgc

SEQ ID NO: 191 - DNA, 28 bp, synthetic construct
  source 1..28: mol_type = other DNA
aacaagagttattacaacacgaagtgat

SEQ ID NO: 192 - DNA, 27 bp, synthetic construct
  source 1..27: mol_type = other DNA
ttaccgcacttaattctaatacttccg

SEQ ID NO: 193 - DNA, 26 bp, synthetic construct
  source 1..26: mol_type = other DNA
aagaaggctcactatttgattgaacc

SEQ ID NO: 194 - DNA, 28 bp, synthetic construct
  source 1..28: mol_type = other DNA
atgaaacaaatcgttttaaaaacaagct

SEQ ID NO: 195 - DNA, 18 bp, synthetic construct
  source 1..18: mol_type = other DNA
ttgtcgggtcctgtaggg

SEQ ID NO: 196 - DNA, 28 bp, synthetic construct
  source 1..28: mol_type = other DNA
aatggcaagatttatggtgaaatctaca

SEQ ID NO: 197 - DNA, 27 bp, synthetic construct
  source 1..27: mol_type = other DNA
cctttaaagcccctctagaacaagaaa

SEQ ID NO: 198 - DNA, 28 bp, synthetic construct
  source 1..28: mol_type = other DNA
gaataccgtaacgttatctttatctaca

SEQ ID NO: 199 - DNA, 19 bp, synthetic construct
  source 1..19: mol_type = other DNA
ttcgctgcaatcggttcat

SEQ ID NO: 200 - DNA, 25 bp, synthetic construct
  source 1..25: mol_type = other DNA
atgaatcaagcggtcacagaaaaga

SEQ ID NO: 201 - DNA, 19 bp, synthetic construct
  source 1..19: mol_type = other DNA
tgatagccatgcaatgcca

SEQ ID NO: 202 - DNA, 24 bp, synthetic construct
  source 1..24: mol_type = other DNA
ggcaatagatcgtgattttggtcg

SEQ ID NO: 203 - DNA, 21 bp, synthetic construct
  source 1..21: mol_type = other DNA
tttgatgtggtaggactgcca

SEQ ID NO: 204 - DNA, 28 bp, synthetic construct
  source 1..28: mol_type = other DNA
cagagaggtgaaaaatacccctaaataa

SEQ ID NO: 205 - DNA, 22 bp, synthetic construct
  source 1..22: mol_type = other DNA
agtttcattttcgcctaaccca

SEQ ID NO: 206 - DNA, 28 bp, synthetic construct
  source 1..28: mol_type = other DNA
tgacattgaataactacttattgacaca

SEQ ID NO: 207 - DNA, 28 bp, synthetic construct
  source 1..28: mol_type = other DNA
ggacatatcaaccttaggtgtaagttcc

SEQ ID NO: 208 - DNA, 28 bp, synthetic construct
  source 1..28: mol_type = other DNA
atgaatattgcaacaaaattaatggcta

SEQ ID NO: 209 - DNA, 27 bp, synthetic construct
  source 1..27: mol_type = other DNA
ggtattttcggtaaaaatggcgaagta

SEQ ID NO: 210 - DNA, 28 bp, synthetic construct
  source 1..28: mol_type = other DNA
tttttatcttcttttgtggttgccgtag

SEQ ID NO: 211 - DNA, 28 bp, synthetic construct
  source 1..28: mol_type = other DNA
aaaaatatggctccacaaatgggtaatc

SEQ ID NO: 212 - DNA, 26 bp, synthetic construct
  source 1..26: mol_type = other DNA
tttccgcattacttttatccttactt

SEQ ID NO: 213 - DNA, 28 bp, synthetic construct
  source 1..28: mol_type = other DNA
cttctacagaagcctttaatttctgatt

SEQ ID NO: 214 - DNA, 28 bp, synthetic construct
  source 1..28: mol_type = other DNA
gcgatgaaaaagttagtttgaatgaata

SEQ ID NO: 215 - DNA, 28 bp, synthetic construct
  source 1..28: mol_type = other DNA
aataaaccggacggaaataatgggaata

SEQ ID NO: 216 - DNA, 28 bp, synthetic construct
  source 1..28: mol_type = other DNA
catctaaatctaaagctaaaggatcgta

SEQ ID NO: 217 - DNA, 28 bp, synthetic construct
  source 1..28: mol_type = other DNA
cgataagttattatctttttggctacct

SEQ ID NO: 218 - DNA, 25 bp, synthetic construct
  source 1..25: mol_type = other DNA
caaggattttatgaaaactgggcgg

SEQ ID NO: 219 - DNA, 23 bp, synthetic construct
  source 1..23: mol_type = other DNA
ggatttactcgaacgcgcctata

SEQ ID NO: 220 - DNA, 25 bp, synthetic construct
  source 1..25: mol_type = other DNA
ccaagaggttcaataccaaagtttt

SEQ ID NO: 221 - DNA, 27 bp, synthetic construct
  source 1..27: mol_type = other DNA
tgctcattcgataaacgaatattttct

SEQ ID NO: 222 - DNA, 28 bp, synthetic construct
  source 1..28: mol_type = other DNA
actgatattaatttatccgaactttggt

SEQ ID NO: 223 - DNA, 24 bp, synthetic construct
  source 1..24: mol_type = other DNA
gcttcaaaaacaggctcaagtgta

SEQ ID NO: 224 - DNA, 25 bp, synthetic construct
  source 1..25: mol_type = other DNA
atgtcaaatgccaaagcttacatcc

SEQ ID NO: 225 - DNA, 23 bp, synthetic construct
  source 1..23: mol_type = other DNA
gtatcgacttcgacctcttggtg

SEQ ID NO: 226 - DNA, 22 bp, synthetic construct
  source 1..22: mol_type = other DNA
gccatcgtagttccatacagaa

SEQ ID NO: 227 - DNA, 20 bp, synthetic construct
  source 1..20: mol_type = other DNA
gttagcacctgcaaggtagt

SEQ ID NO: 228 - DNA, 24 bp, synthetic construct
  source 1..24: mol_type = other DNA
cttgtcatggactcgtgaagaagt

SEQ ID NO: 229 - DNA, 27 bp, synthetic construct
  source 1..27: mol_type = other DNA
gatcggttacatgtacggtcaatacaa

SEQ ID NO: 230 - DNA, 22 bp, synthetic construct
  source 1..22: mol_type = other DNA
agtaaaccaaaccgtaaccagt

SEQ ID NO: 231 - DNA, 24 bp, synthetic construct
  source 1..24: mol_type = other DNA
cctgctcgttcaaccaattctttc

SEQ ID NO: 232 - DNA, 28 bp, synthetic construct
  source 1..28: mol_type = other DNA
actgtcgatgatgttttggactatttta

SEQ ID NO: 233 - DNA, 25 bp, synthetic construct
  source 1..25: mol_type = other DNA
aaaggaaaagagcccttcaatctca

SEQ ID NO: 234 - DNA, 20 bp, synthetic construct
  source 1..20: mol_type = other DNA
gaaaacgggatgaccatcct

SEQ ID NO: 235 - DNA, 19 bp, synthetic construct
  source 1..19: mol_type = other DNA
gagacggacacgagaaacg

SEQ ID NO: 236 - DNA, 19 bp, synthetic construct
  source 1..19: mol_type = other DNA
aggccattgcccagaaaat

SEQ ID NO: 237 - DNA, 22 bp, synthetic construct
  source 1..22: mol_type = other DNA
gagcagtttatcgcgttcctga

SEQ ID NO: 238 - DNA, 23 bp, synthetic construct
  source 1..23: mol_type = other DNA
aaaaatgagcaagaacacaaggc

SEQ ID NO: 239 - DNA, 28 bp, synthetic construct
  source 1..28: mol_type = other DNA
tatttcaacaagtgtgcaagcattactg

SEQ ID NO: 240 - DNA, 27 bp, synthetic construct
  source 1..27: mol_type = other DNA
atggtagttaaagttggtattaacggt

SEQ ID NO: 241 - DNA, 27 bp, synthetic construct
  source 1..27: mol_type = other DNA
gaagtactcaagagtacgaacaagttg

SEQ ID NO: 242 - DNA, 22 bp, synthetic construct
  source 1..22: mol_type = other DNA
agttatcgaagttgatggcgag

SEQ ID NO: 243 - DNA, 25 bp, synthetic construct
  source 1..25: mol_type = other DNA
cctgagataactgtttcagtaccat

SEQ ID NO: 244 - DNA, 20 bp, synthetic construct
  source 1..20: mol_type = other DNA
catccacgctaacggtgcta

SEQ ID NO: 245 - DNA, 21 bp, synthetic construct
  source 1..21: mol_type = other DNA
acagaacctgttggaactgga

SEQ ID NO: 246 - DNA, 23 bp, synthetic construct
  source 1..23: mol_type = other DNA
ctgcaaacatcgttcctaactca

SEQ ID NO: 247 - DNA, 28 bp, synthetic construct
  source 1..28: mol_type = other DNA
tacagagaaattagatagcactactaca

SEQ ID NO: 248 - DNA, 25 bp, synthetic construct
  source 1..25: mol_type = other DNA
cttaaatatgagtgtgttgatggca

SEQ ID NO: 249 - DNA, 28 bp, synthetic construct
  source 1..28: mol_type = other DNA
atggatatatttctgatactctagcaga

SEQ ID NO: 250 - DNA, 27 bp, synthetic construct
  source 1..27: mol_type = other DNA
cttttcaatcttgttctccagaatcta

SEQ ID NO: 251 - DNA, 27 bp, synthetic construct
  source 1..27: mol_type = other DNA
aaaagtttctcaattcagcaattcaat

SEQ ID NO: 252 - DNA, 27 bp, synthetic construct
  source 1..27: mol_type = other DNA
ttgcaaaattcttggttctctagaaat

SEQ ID NO: 253 - DNA, 28 bp, synthetic construct
  source 1..28: mol_type = other DNA
tgagtaagaactttaatgtcaactacgt

SEQ ID NO: 254 - DNA, 28 bp, synthetic construct
  source 1..28: mol_type = other DNA
atgacaatccacaaagtagcaatcaatg

SEQ ID NO: 255 - DNA, 28 bp, synthetic construct
  source 1..28: mol_type = other DNA
aatgaaacccttgcttatgtagaagatc

SEQ ID NO: 256 - DNA, 28 bp, synthetic construct
  source 1..28: mol_type = other DNA
gaagactcattatcatatcatgcataca

SEQ ID NO: 257 - DNA, 27 bp, synthetic construct
  source 1..27: mol_type = other DNA
ggtaatttgagtatggtttacgttgta

SEQ ID NO: 258 - DNA, 27 bp, synthetic construct
  source 1..27: mol_type = other DNA
gttcaggaagattcttaactaaggaag

SEQ ID NO: 259 - DNA, 28 bp, synthetic construct
  source 1..28: mol_type = other DNA
aattaattctggattcatgacaacagtt

SEQ ID NO: 260 - DNA, 25 bp, synthetic construct
  source 1..25: mol_type = other DNA
tgctcctgtagttgtaggaataatt

SEQ ID NO: 261 - DNA, 19 bp, synthetic construct
  source 1..19: mol_type = other DNA
aggaaacaggtggtcgact

SEQ ID NO: 262 - DNA, 21 bp, synthetic construct
  source 1..21: mol_type = other DNA
ccagagtggatttccgttggt

SEQ ID NO: 263 - DNA, 20 bp, synthetic construct
  source 1..20: mol_type = other DNA
gcatttgccatgagaggagg

SEQ ID NO: 264 - DNA, 23 bp, synthetic construct
  source 1..23: mol_type = other DNA
tctcttcactgtcacgtacgaca

SEQ ID NO: 265 - DNA, 20 bp, synthetic construct
  source 1..20: mol_type = other DNA
gcagcgtctcttcctctttt

SEQ ID NO: 266 - DNA, 25 bp, synthetic construct
  source 1..25: mol_type = other DNA
gaagaagggtacgtgttgcatcata

SEQ ID NO: 267 - DNA, 21 bp, synthetic construct
  source 1..21: mol_type = other DNA
cacctagtatcgtgcggcaat

SEQ ID NO: 268 - DNA, 22 bp, synthetic construct
  source 1..22: mol_type = other DNA
gagagacgctctacaaaatcca

SEQ ID NO: 269 - DNA, 16 bp, synthetic construct
  source 1..16: mol_type = other DNA
tcggctccgcggtctc

SEQ ID NO: 270 - DNA, 21 bp, synthetic construct
  source 1..21: mol_type = other DNA
agggaggaagacgaaagttgt

SEQ ID NO: 271 - DNA, 20 bp, synthetic construct
  source 1..20: mol_type = other DNA
cttctccctctccgactctc

SEQ ID NO: 272 - DNA, 21 bp, synthetic construct
  source 1..21: mol_type = other DNA
acagaagggacagaagtcgaa

SEQ ID NO: 273 - DNA, 19 bp, synthetic construct
  source 1..19: mol_type = other DNA
tgaaggcgagggtgaggat

SEQ ID NO: 274 - DNA, 19 bp, synthetic construct
  source 1..19: mol_type = other DNA
tcgtctcgtctggatcgca

SEQ ID NO: 275 - DNA, 17 bp, synthetic construct
  source 1..17: mol_type = other DNA
ccctcctcctcccttcg

SEQ ID NO: 276 - DNA, 18 bp, synthetic construct
  source 1..18: mol_type = other DNA
gctgcttttcctggaggg

SEQ ID NO: 277 - DNA, 28 bp, synthetic construct
  source 1..28: mol_type = other DNA
ttcaaaaggttttgttgctgatttgtta

SEQ ID NO: 278 - DNA, 22 bp, synthetic construct
  source 1..22: mol_type = other DNA
aatgaagacagaagcgcacatc

SEQ ID NO: 279 - DNA, 28 bp, synthetic construct
  source 1..28: mol_type = other DNA
aaaagtggaccatatgtatcttgtttat

SEQ ID NO: 280 - DNA, 28 bp, synthetic construct
  source 1..28: mol_type = other DNA
gaaaagatggaagcacttatagtaacta

SEQ ID NO: 281 - DNA, 27 bp, synthetic construct
  source 1..27: mol_type = other DNA
catttggtgtttcagaagcttggttaa

SEQ ID NO: 282 - DNA, 25 bp, synthetic construct
  source 1..25: mol_type = other DNA
acgtcatcgaagtttttggttttat

SEQ ID NO: 283 - DNA, 20 bp, synthetic construct
  source 1..20: mol_type = other DNA
gctcgctgtgtgcctaatac

SEQ ID NO: 284 - DNA, 28 bp, synthetic construct
  source 1..28: mol_type = other DNA
gcgataaataactatatccggtattagc

SEQ ID NO: 285 - DNA, 18 bp, synthetic construct
  source 1..18: mol_type = other DNA
gtcgtccccaccttcctc

SEQ ID NO: 286 - DNA, 26 bp, synthetic construct
  source 1..26: mol_type = other DNA
ctcgtgtcgtgagatgttaggttaag

SEQ ID NO: 287 - DNA, 21 bp, synthetic construct
  source 1..21: mol_type = other DNA
cgcctattggtgttcttccta

SEQ ID NO: 288 - DNA, 28 bp, synthetic construct
  source 1..28: mol_type = other DNA
tttttgctaagtctggagttaaatgctg

SEQ ID NO: 289 - DNA, 28 bp, synthetic construct
  source 1..28: mol_type = other DNA
agatctttatattcataacgggagacac

SEQ ID NO: 290 - DNA, 21 bp, synthetic construct
  source 1..21: mol_type = other DNA
tgcagcaatgaatcaaggact

SEQ ID NO: 291 - DNA, 28 bp, synthetic construct
  source 1..28: mol_type = other DNA
gaattatccgggaaattgcactaaaagt

SEQ ID NO: 292 - DNA, 24 bp, synthetic construct
  source 1..24: mol_type = other DNA
acagttccactaccgataactttt

SEQ ID NO: 293 - DNA, 24 bp, synthetic construct
  source 1..24: mol_type = other DNA
ggcgagaaactggatattcaagtt

SEQ ID NO: 294 - DNA, 27 bp, synthetic construct
  source 1..27: mol_type = other DNA
ggtgaacatggtgattcatcttttgtt

SEQ ID NO: 295 - DNA, 25 bp, synthetic construct
  source 1..25: mol_type = other DNA
tggactgagccaaaattagattttg

SEQ ID NO: 296 - DNA, 25 bp, synthetic construct
  source 1..25: mol_type = other DNA
gcatttgaacagcttttaatttcgc

SEQ ID NO: 297 - DNA, 28 bp, synthetic construct
  source 1..28: mol_type = other DNA
actttagaatgatttcatcttgattggt

SEQ ID NO: 298 - DNA, 25 bp, synthetic construct
  source 1..25: mol_type = other DNA
aaacttcctttctctgcaaatgatt

SEQ ID NO: 299 - DNA, 28 bp, synthetic construct
  source 1..28: mol_type = other DNA
ttagcctctctgattattgatatggatc

SEQ ID NO: 300 - DNA, 28 bp, synthetic construct
  source 1..28: mol_type = other DNA
acttaaatagacagttccgattttggta

SEQ ID NO: 301 - DNA, 22 bp, synthetic construct
  source 1..22: mol_type = other DNA
tttggcagcagttaaaactgtt

SEQ ID NO: 302 - DNA, 28 bp, synthetic construct
  source 1..28: mol_type = other DNA
tattcttatctaaatcagatcccgagtc

SEQ ID NO: 303 - DNA, 1941 bp, synthetic construct
  source 1..1941: mol_type = other DNA
atggcatcaggaggagctttttgtcttattgctaacgatgggaaggccgacaagattatattggcccaagacttgctgaatagcaggatctctaacattaaaaatgtgaacaaaagttatgggaaacccgatcccgaacccactttgagtcaaatcgaagaaacacatttggtgcattttaatgcgcattttaagccttatgttccagtagggtttgaatacaataaagtacgcccgcatacgggtacccccaccttgggaaacaagcttacctttggtattccccagtacggagactttttccatgatatggtgggccatcatatattgggtgcatgtcattcatcctggcaggatgctccgattcagggcacgtcccagatgggggcccatgggcagcttcaaacgtttcctcgcaacggatatgactgggacaaccaaacacccttagagggcgccgtttacacgcttgtagatccttttggaagacccattgtacccggcacaaagaatgcgtaccgaaacttggtttactactgcgaataccccggagaacgactttatgaaaacgtaagattcgatgtaaatggaaattccctagacgaatatagttcggatgtcacaacgcttgtgcgcaaattttgcatcccaggggataaaatgactggatataagcacttggttggccaggaggtatcggtggagggaaccagtggccctctcctatgcaacattcatgatttgcacaagccgcaccaaagcaaacctattcttaccgatgaaaatgatacgcagcgaacgtgtagccataccaacccgaaatttctttcacagcattttcccgagaactctcacaatatccaaacagcaggtaaacaagatattactcctatcacggacgcaacgtatctggacataagacgtaatgttcattacagctgtaatggacctcaaacccctaaatactatcagccccctcttgcgctctggattaagttgcgcttttggtttaatgagaacgtgaaccttgctattccctcagtatccattcccttcggcgagcgctttatcaccataaagcttgcatcgcaaaaggatttggtgaatgaatttcctggactttttgtacgccagtcacgttttatagctggacgccccagtagacgcaatatacgctttaaaccatggtttatcccaggagtcattaatgaaatctcgctcacgaataatgaactttacatcaataacctgtttgtaacccctgaaatacacaacctttttgtaaaacgcgttcgcttttcgctgatacgtgtccataaaacgcaggtgacccacaccaacaataaccaccacgatgaaaaactaatgtctgctcttaaatggcccattgaatatatgtttataggattaaaacctacctggaacatctccgatcaaaatcctcatcaacaccgagattggcacaagttcggacatgttgttaacgccattatgcagcccactcaccacgcagagataagctttcaggatagagatacagctcttccagacgcatgttcatctatatctgatattagccccgttacgtatccgatcacattacctattattaaaaacatttccgtaactgctcatggtatcaatcttatcgataaatttccatcaaagttctgcagctcttacatacccttccactacggaggcaatgcgattaaaacccccgatgatccgggtgcgatgatgattacctttgctttgaagccacgggaggaataccaacccagtggtcatattaacgtatccagagcaagagaattttatattagttgggacacggattacgtggggtctatcactacggctgatcttgtggtatcggcatctgctattaactttcttcttcttcagaacggttcagctgtgctgcgttacagtacctaa

SEQ ID NO: 304 - DNA, 555 bp, synthetic construct
  source 1..555: mol_type = other DNA
atggattctgaattttttcaaccggtttatccgcggcattatggtgagtgtttgtcaccagtcactacaccaagcttcttctccacacatatgtatactattctcattgctatcgtggtcttagtcatcattatcatcgttctaatctatctattctcttcaagaaagaaaaaagctgctgctattgaggaggaagatatacagtttataaatccttatcaagatcagcagtgggtagaagtcactccacaaccaggtacctctaaaccagctggagcgactacagcaagtgtaggcaagccagtcacgggcagaccggcaacaaacagaccagcaacaaacaaaccagttacggacaacccagttacggacagactagtcatggcaactggcgggccggcggccgcacctgcggccgcgagtgctcctgctcatccggctgagccttacacgacagtcactactcagaacactgcttcacaaacaatgtcggctattgaaaatttacgacaaagaaacacctatacgcataaagacctagaaaactccttgtaa

SEQ ID NO: 305 - DNA, 1083 bp, synthetic construct
  source 1..1083: mol_type = other DNA
atgataatacttatttttttaatattttctaacatagttttaagtattgattattgggttagttttaataaaacaataattttagatagtaatattactaatgataataatgatataaatggagtatcatggaatttttttaataattcttttaatacactagctacatgtggaaaagcaggtaacttttgtgaatgttctaattatagtacatcaatatataatataacaaataattgtagcttaactatttttcctcataatgatgtatttgatacaacatatcaagtagtatggaatcaaataattaattatacaataaaattattaacacctgctactcccccaaatatcacatataattgtactaattttttaataacatgtaaaaaaaataatggaacaaacactaatatatatttaaatataaatgatacttttgttaaatatactaatgaaagtatacttgaatataactggaataatagtaacattaacaattttacagctacatgtataattaataatacaattagtacatctaatgaaacaacacttataaattgtacttatttaacattgtcatctaactatttttatactttttttaaattatattatattccattaagcatcataattgggataacaataagtattcttcttatatccatcataacttttttatctttacgaaaaagaaaaaaacatgttgaagaaatagaaagtccaccacctgaatctaatgaagaagaacaatgtcagcatgatgacaccacttccatacatgaaccatctcccagagaaccattacttcctaagccttacagtcgttatcagtataatacacctatttactacatgcgtccctcaacacaaccactcaacccatttcccttacctaaaccgtgtcctccacccaaaccatgtccgccacccaaaccatgtcctccacctaaaccatgtccttcagctgaatcctattctccacccaaaccactacctagtatcccgctactacccaatatcccgccattatctacccaaaatatttcgcttattcacgtagatagaattatttaa

SEQ ID NO: 306 - DNA, 1596 bp, synthetic construct
  source 1..1596: mol_type = other DNA
atgttctctctccagaacttatgtcgaaaaacattacctaaccgtaaacttcctgaattttttgacgaatatatattacaactgctgggattatactgggaaaaccatggaactattcaacgagcaggaaacaactgtgtgcttatacagcaacataccctcattcccgtaaatgaagccctgagaacagcagcatctgaagaaaattatgagatcgtgagccttttattagcgtgggaggggaacctttactatgctattataggggctctagagggcaaccgccacgacttaattcgtaaatatgatgaccaaatcaaggaccatcatgaaattctgccattcattgacgatccagtcatatttcacaaatgccatatcatgcggcaatgcttttttgattgtattttatatcaagctgtaaaatatagtaagtttcgcgttcttctttactttaaacatagattagaggatgatttgcccttcactcatttacttattgaaaaggcatgtaaagatcataattatgaagttattaaatggatatatgaaaacctacatatctacaatatgatagatacctttgaatgtgctattgcccataaggatctacatctatattgtttggggtatagatttatatataacagaatcgtacccgataagtatcatcatttagatattcgcatgctttcaagcctacaactcctacataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaagcaaaatcttccaggaaaaccttgaacttactactgtctcacctaaacctttccatcaacctcatcaaaaaaataagccattatgttgccacttacaattcaacaaatataataggcattctgagtatgcggcggaaaaagaagatatatttagatatcatattgacaaaatttgtaaaaaaagctatttttaataagtttgtcgttcgatgtatggatacattttctataaacccggaaagaatccttaaaatagccgcgcgaataaataggatgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatttattctaaacaatacatgccttag

SEQ ID NO: 307 - DNA, 1581 bp, synthetic construct
  source 1..1581: mol_type = other DNA
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcaaaccatacatgtttacgacctaaagattatgtttaatattgccatctccaagagggatctgactatgtactccttaggatatattttcctttttgatagagggaacaccgaagctacgttgctaacgcaacatctcaagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggccgtgcaggaaaaggcttctaaaaaaacattgaacttactgttgtcacatttaaactactccgtgaaacgcatcaaaaaactaccgcgctatgtgatagagtacgagtccaccttggtgataaagattttattaaaaaaaagagtgaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgttttatttagccaagctctatgttgctcaaaacgcggccacccaattcagagacatttgtaaggactgttacaaactggatgtggcacggtttaaaccgcggtttaagcaactaatattagactgtttagaaattattactaaaaaatcttgctatagtatcctggaaatcttagaaaaacatattatttccctgtttactatgaaagttatgactgaagaagaaaaaaacctatgtttagaaatattatataaagtaattcattataaaacaatacaatgttaa

SEQ ID NO: 308 - DNA, 1062 bp, synthetic construct
  source 1..1062: mol_type = other DNA
atgtcgttgccgctctctctgcagaccctcgtcaaaaagacgatagccagccagtgtttgtcaatagatgaacactgcattttgaaatattgtggcctatggtggcatgatgctcctctcaagctttgtatggatcgtggccgaatacaaataaaatcaggatttttaggagaagatatagaccttcgtgtggcattaataatagctgttaaggaaaacaactatagtctgataaagctctttacagagtggggcgcaaatatcaactatggtttgctttctatcaatacgaagcacatccgagagttgtgtagacagctaggcgccaaagaaactctagaggacaacgatattttccgtatttttaccaggataatgcacaataaaaccagcggcagtattattttgtgccatgaaatttttatgaataatcctattttagaaaacaaatttgttatacaattaaggggcttaatttataaaagactatgggggctcatagaaataaaagaaacggacgagttaaatggtttactagtgaagtattggtacgccaaagcagtacaatacgattgtaaggacgccatttgttttctagatgagaaatatacggatcttaatgaatggcgattaaaatgtctcctgtattataacaaaatatatgagcttcatgagatgtaccacaaggaaaacatccaaatagacgtccatgacatgatatgtctggcttctaccaaggataacaatccattaacaatatattactgttacgcgctggggggcaacatcaaccaagctatgcttacttcagtacaatattataacatcggtaatatatttttctgtatagatttgggtggtaatgcctttgaagagggtcgtgccatagcggaacaaaaaggttataattttctgagccatagtttggctttggatatttacagctcagatgcttccttgccactaaacttaaaggaccccgaagaaataagcagtttattaaaagattataaatcaaaaaacttatccatcatttgggaatattctcataatatactatag

SEQ ID NO: 309 - DNA, 864 bp, synthetic construct
  source 1..864: mol_type = other DNA
atgttgtctttacaaacgttggccaaaaaagttgtggcatgcaattatctttcaagtgactatgattatacgttgcagcgttttggtttgtggtgggatttaggtcctattcacctatgtaacaattgtaagcaagttttttcgtataaacatttacagtgtttttctgaggatgatctttgtctcgaagcggcgctagtaaaggccgtgaagagcgataatcttgaacttatacgtttatttgtggattggggcgcaaatcctgaatatgggcttatacgtgttcctgccgtgtatctaaagcggctgtgtgcggaactgggaggcttaacgcctgtatccgaaccccgtcttctggaaattttaaaagaagtggccaggctaaaatcctgtgcaggagttctgctgggttatgacatgttttgtcataatccactcttggaaaccgtaactagaaccactttagacacagttacgtacacctgttcaaacattccgttgacgggggatacggcgcaccacctattaacaaagttttggtttgccctggcattacgacataattttacaaaggctattcactatttctataaaaggcataaaaatcacctctattggcgggtagcttgtagcctttattttaataacatttttgacatacacgagttgtgtcgtgaaaaagagatttgcatcagccctaatctgatgatgaaatttgcttgcttgcgggaaaaaaattacgcggccatttattactgtcataggttgggggctagtctcgattatggcatgaatctttctatctataacaataatactttaaacatgtttttctgtattgatttggggggctgccgattttga

SEQ ID NO: 310 - DNA, 618 bp, synthetic construct
  source 1..618: mol_type = other DNA
atggttgagccacgcgaacagttttttcaagacctgctttcagcagtggatcaacaaatggacactgtaaaaaatgacataaaagacatcatgaaagaaaaaacatcttttatggtgtcattcgaaaactttatagaacgttacgataccatggaaaaaaatattcaagaccttcagaataagtacgaagaaatggcggccaaccttatgaccgtcatgacggatacaaaaattcagcttggagccattatcgcccaacttgagattctgatgataaatggcactccacttccggcaaaaaaaacaacgattaaggaggctatgcccctaccttcatcaaacacgaacaatgatcaaacgagtcctcccgcctcaggcaaaacaagtgaaacacctaaaaaaaatcccacgaatgcaatgttcttcacgcgtagcgaatgggcatcctcgaaaacttttcgagaaaagtttttaacaccagaaattcaggccatattggatgagcagtttgcaaacaagaccgggatcgaaagattgcatgccgagggtctttacatgtggagaacccaattctctgacgaacagaagaaaatggtcaaagagatgatgaagaagtaa

SEQ ID NO: 311 - DNA, 525 bp, synthetic construct
  source 1..525: mol_type = other DNA
atggggtcgtctctagacgacttctgcaatgatagcacagctccacagaaggtgcttttggcgttttccattacctacacgccagtgatgatatatgctctaaaggtaagtcgcggccgactgctagggcttctgcaccttttgatctttctgaattgtgcttttaccttcgggtacatgacattcgtgcactttgagagcacaaatagggtcgcgctcactatgggagcagtagttgcacttctttggggagtgtactcagccatagaaacctggaaattcatcacctccagatgtcgtttgtgcttgctaggccgcaagtacattctggcccctgcccaccacgtcgaaagtgccgcgggctttcatccgattgcggcaaatgataaccacgcatttgtcgtccggcgtcccggctccactacggtcaacggcgcattggtgcccgggttgaaaagcctcgtgttgggtggcagaaaagctgttaagcagggagtggtaaaccttgttaaatatgccaaataa

SEQ ID NO: 312 - DNA, 372 bp, synthetic construct
  source 1..372: mol_type = other DNA
atgccaaataacaacggcagacagcaaaataaaaagaagggggatggccagccagtcaatcagctgtgccagatgctgggcaaaattatcgcccagcagaaccagtccagaggtaagggaccggggaagaagaataagaatagaaacccggagaagccccatttccctctagcgactgaagatgacgtcagacatcacttcaaccccagtgaacgacaattgtgcctgtcgtcaatccggactgcctttaaccaaggcgctgggacttgcaccctgtcagactcagggagaataagttacactgtggagtttagtttgcctactcatcacactgtgcgcttgattcgcgccacagcgtcaccctcagcatga

SEQ ID NO: 313 - DNA, 522 bp, synthetic construct
  source 1..522: mol_type = other DNA
atgggaagcctagatgatttctgcttcgatcacaccgccgcacaaaagcttgtgctagcctttagcatcacatatacacctataatgatatacgccctcaaggtgtcacgcggccgactcctggggctgttgcacatcctgatatttctgaactgtgccttcacgttcggatacatgacatatgtgcattttgaatccaccaaccgtgtcgcactcactatgggggctgttgttgcccttctgtggggtatttatagcttcacagagtcatggaagttcattacttccagatgcagattgtgttgcctaggccggcgatacattctggcccctgcccaccacgtagaaagtgctgcaggtctccattcaatcccagcgtctggtaaccgagcatacgctgtgagaaagcccggactaacatcagtgaacggcaccctagtaccagggcttcggagcctcgtgttgggcggcaaacgagctgttaaacgaggagtggttaacctcgtcaagtatggccggtaa

SEQ ID NO: 314 - DNA, 387 bp, synthetic construct
  source 1..387: mol_type = other DNA
atggccggtaagaaccagagccagaagaaaaggaaaaacacagctccaatggggaatggccagccagtcaatcaactgtgtcagttgctgggtgcaatgataaagtcccagcgccagcaatctaggggaggacaggccaaaaagaaaaagcctgagaagccgcattttcccctggccgctgaagatgacatccggcaccatctcacccagaccgaacgatccctttgcttgcaatcgatccagacggcttttaatcaaggcgcaggaactgcgtcgctttcatccagcgggaaggttggttttcaggttgagtttatgctgccggttgctcatacagtgcgcctgattcgcgtgacttctacatccgccagtcagggtgcaagttaa

SEQ ID NO: 315 - DNA, 1677 bp, synthetic construct
  source 1..1677: mol_type = other DNA
atgcggccctttctgctgcgcgccgcgcagctcctggcgctgctggccctggcgctctccaccgaggccccgagcctctccgccgagacgaccccgggccccgtcaccgaggtcccgagtccctcggccgaggtctgggacgacctctccaccgaggccggcgacgatgacctcaacggcgacctcgacggcgacgaccgccgcgcgggcttcggctcggccctcgcctccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccacccggacctggtgctgggccgcgcctgcgtccccgaggccccggagatgggcatcggcgactacctgccgcccgaggtgccgcggctccggcgcgagccgcccatcgtcaccccggagcggtggtcgccgcacctgagcgtcctgcgggccacgcccaacgacacgggcctctacacgctgcacgacgcctcggggccgcgggccgtgttctttgtggcggtgggcgaccggccgcccgcgccggcggacccggtgggccccgcgcgccacgagccccgcttccacgcgctcggcttccactcgcagctcttctcgcccggggacacgttcgacctgatgccgcgcgtggtctcggacatgggcgactcgcgcgagaactttaccgccacgctggactggtactacgcgcgcgcgcccccgcggtgcctgctgtactacgtgtacgagccctgcatctaccacccgcgcgcgcccgagtgcctgcgcccggtggacccggcgtgcagcttcacctcgccggcgcgcgcgcggctggtggcgcgccgcgcgtacgcctcgtgcagcccgctgctcggggaccggtggctgaccgcctgccccttcgacgccttcggcgaggaggtgcacacgaacgccaccgcggacgagtcggggctgtacgtgctcgtgatgacccacaacggccacgtcgccacctgggactacacgctcgtcgccaccgcggccgagtacgtcacggtcatcaaggagctgacggccccggcccgggccccgggcaccccgtggggccccggcggcggcgacgacccgatctacgtggacggcgtcacgacgccggcgccgcccgcgcgcccgtggaacccgtacggccggacgacgcccgggcggctgtttgtgctggcgctgggctccttcgtgatgacgtgcgtcgtcggggggccgtctggctctgcgtgctgtgctcccggcgccgggcggcctcgcggccgttccgggtgccgacgcgggcgcggacgcacatgctctctccggtgtacaccagcctgcccacgcacgaggactactacgacggcgacgacgacgacgaggaggcgggcgtcatccgccggcggcccgcctcccccggcggagacagcggctacgaggggtcgtacgcgagcctggaccccgaggacgagttcagcagcgacgaggacgacgggctgtacgtgcgccccgaggaggcgccccgctccggcttcgacgtctggttccgcgatccggagaaaccggaagtga

SEQ ID NO: 316 - DNA, 2745 bp, synthetic construct
  source 1..2745: mol_type = other DNA
atgcccgctggtggcggtctttggcgcgggccccgcgggcatcggcccgggcaccacggcggtgctggcctcggacgtctttggcctgctccacaccacgctgcagctgcgcggggcgccgtcgcgctagcgctgctgctgctggcgctcgccgcgaccccgacgtgcggcgcggcggccgtgacgcgggccgcctcggcctcgcccgcgcccgggacgggcgccaccccagacggcttctccgcggaggagtccctcgaggagatcgacggggccgtcacccccggcccctcgaacgcccccgacggcgagtacggcgacctggtcgcgcgcacggccgtgcgcgcggccgcgaccgagcgggaccgcttctacgtctgcccgccgccgtccggctccacggtggtgcgcctggagcccgagcaggcctgccccgagtactcgcaggggcgcaacttcacggaggggatcgccgtgctcttcaaggagaacatcgccccgcacaagttcaaggcccacatctactacaagaacgtcatcgtcacgaccgtgtggtccgggagcacgtacgcggccatcacgaaccgcttcacggaccgcgtgcccgtccccgtgcaggagatcacggacgtgatcgaccgccgcggcaagtgcgtctccaaggccgagtacgtgcgcaacaaccacaaggtgaccgccttcgactgcgacgagaaccccgtcgaggtgggcctgcgcccctcgcgcctgaacgcgctcggcacccgcggctggcacaccaccaacgacacctacaccaagatcggcgccgcgggcttctaccacacgggcacctccgtcaactgcatcgtcgaggaggtggaggcgcgctccgtgtacccctacgactccttcgccctgtccacgggggacatcgtgtacatgtcccccttctacggcctgcgcgagggggcccacggggagcacatcggctacgcgcccgggcgcttccagcaggtggagcactactaccccatcgacctggactcgcgcctccgcgcctccgagagcgtgacgcgcaactttctgcgcacgccgcacttcacggtggcctgggactgggcccccaagacgcggcgcgtgtgcagcctggccaagtggcgcgaggccgaggagatgatccgcgacgagacgcgcgacgggtccttccgcttcacgtcgcgggccctgggcgcctccttcgtcagcgacgtcacgcagctcgacctgcagcgcgtgcacctgggcgactgcgtcctccgcgaggcctcggaggccatcgacgccatctaccggcggcgctacaacaacacgcacgtgctggccggcgacaagcccgaggtgtacctcgcccgcgggggcttcgtggtggccttccgcccgctgatctcgaacgagctggcgcagctgtacgcgcgcgagctcgagcgcctcggcctcgccggcgtcgtgggccccgcgtcccccgcggccgcccgtcgggcccggcgctcccccggcccggcggggacgcccgagccgccggccgtcgacggcacggggcacctgcgcatcaccacgggctcggccgagtttgcgcgcctgcagttcacctacgaccacatccaggcgcacgtgaacgacatgctgagccgcatcgcggccgcctggtgcgagctgcagaacaaggaccgcaccctgtggggcgagatgtcgcgcctgaaccccagcgccgtggccacggccgcgctgggccagcgcgtctcggcgcgcatgctcggcgacgtgatggccatctcgcggtgcgtggaggtgcgcggcggcgtgtacgttcagaactccatgcgcgtgcccggcgagcgcggcacgtgctacagccgcccgctggtgaccttcgagcacaacggcacgggcgtgatcgagggccagctcggcgacgacaacgagctcctcatctcgcgcgacctcatcgagccctgcaccggcaaccaccggcgctactttaagctgggcggcgggtacgtgtactacgaggactactgctacgtgcgcatggtggaggtgcccgagacgatcagcacgcgggtgaccctgaacctgacgctgctcaaggaccgcgagttcctgccccccgaggtgtacacgcgcgaggagctcgccgacacgggcctcctggactacagcgagatccagcgccgcaaccagctgcacgcgctcaagttctacgacattgaccgcgtggtcaaggtggaccacaacgtggtgctgctgcgcggcatcgccaacttcttccagggcctcggcgacgtgggcgccgccgtcggcaaggtggtcctgggcgccacgggggccgtgatctcggccgtcggcggcatggtgtccttcccgtccaaccccttcggggcgctcgccatcgggctgctggtgctggccggcctggtcgcggccttcctggcctaccggcacatctcgcgcctgcgccgcaaccccatgaaggccctgtaccccgtcacgacgaaggcgctcaaggaggacggcgtcgaagaggacggcgtggacgaggccaagctggaccaggcccgggacatgatccggtacatgtccatcgtgtcggccctcgagcagcaggagcacaaggcgcgcaagaagaacagcgggcccgcgctgctggccagccgcgtcggggcgatggccacgcgccgccggcactaccagcgcctcgagaacgaggaccccgacgccctgtag

SEQ ID NO: 317 - DNA, 702 bp, synthetic construct
  source 1..702: mol_type = other DNA
atgacgtatccaaggaggcgttaccgaagaagaagacaccgcccccgcagccatcttggccagatcctccgccgccgcccctggctcgtccacccccgccaccgttaccgctggagaaggaaaaatggcatcttcaacacccgcctctcccgcaccttcggatatactgtcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattttagatgataactttgtaacaaaggccacagccctcacctatgacccctatgtaaactactcctcccgccataccataacccagcccttctcctaccactcccggtactttacccccaaacctgtcctagattccactattgattacttccaaccaaacaacaaaagaaaccagctgtggctgagactacaaactgctggaaatgtagaccacgtaggcctcggcactgcgttcgaaaacagtatatacgaccaggaatacaatatccgtgtaaccatgtatgtacaattcagagaatttaattttaaagaccccccacttaacccttaa

SEQ ID NO: 318 - DNA, 945 bp, synthetic construct
  source 1..945: mol_type = other DNA
atgcccagcaagaagagtggaagaagcggaccccaaccacataaaaggtgggtgttcacgctgaataatccttccgaagacgagcgcaagaaaatacgggagctcccaatctccctatttgattattttattgttggcgaggaaggcaatgaggagggccgaacaccccacctacaggggttcgctaattttgtgaagaagcaaacttttaataaagtgaagtggtattttggtgcccgctgccacatcgagaaagcgaaaggaacagatcagcagaataaagaatattgcagtaaagaaggcaacttactgatagaatgtggagctcctagatctcaaggacaacggagcgacctctctaccgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagacgaatgtacacgtcattatggggccacctgggtgtggaaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactctgtgatcgatatcctttgactgttgagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactga

SEQ ID NO: 319 - DNA, 645 bp, synthetic construct
  source 1..645: mol_type = other DNA
atgagacacagagctatattcagaagaagaccccgcccaaggagacgacgacgccacagaaggcgctatgtcagaagaaaactattcattaggaggcccacagctggcacatactacacaaagaaatactccaccatgaacgtcatttccgttggaacccctcagaataacaagccctggcacgccaaccacttcattacccgcctaaacgaatgggaaactgcaattagctttgaatattataagatactaaagatgaaagttacactcagccctgtaatttctccggctcagcaaacaaaaactatgttcgggcacacagccatagatctagacggcgcctggaccacaaacacttggctccaagacgacccttatgcggaaagttccactcgtaaagttatgacttctaaaaaaaaacacagccgttacttcacccccaaaccaattctggcgggaactaccagcgctcacccaggacaaagcctcttctttttctccagacccaccccatggctcaacacatatgaccccaccgttcaatggggagcactgctttggagcatttatgtcccggaaaaaactggaatgacagacttctacggcaccaaagaagtttggattcgttacaagtccgttctctaa

SEQ ID NO: 320 - DNA, 891 bp, synthetic construct
  source 1..891: mol_type = other DNA
gtccggagggaaagcccgaaacacaggtggtgttttacgataaacaactggaccccgaccgagtgggaatctattgtggagtgtggaggcagtatagcgagataccttattatcggcaaagaggttggaaaaagcggtaccccacacttgcaagggtacgtgaatttcaagaacaaaaggcgactcagctcggtgaagcgcttacccggatttggtcgggcccatctggagccggcgagggggagccacaaagaggccagcgagtattgcaagaaagagggggattacctcgagattggcgaagattcctcttcgggtaccagatcggatcttcaagcagcagctcggattctgacggagacgtcgggaaatctgactgaagttgcggagaagatgcctgcagtatttatacgctatgggcggggtttgcgtgatttttgcggggtgatggggttgggtaaaccgcgtgattttaaaactgaagtttatgtttttattggtcctccaggatgcgggaaaacgcgggaagcttgtgcggatgcggctgcgcgggaattgcagttgtatttcaagccacgggggccttggtgggatggttataatggggagggtgctgttattctggatgatttttatgggtgggttccatttgatgaattgctgagaattggggacaggtaccctctgagggttcctgttaagggtgggtttgttaattttgtggctaaggtattatatattactagtaatgttgtaccggaggagtggtattcatcggagaatattcgtggaaagttggaggccttgtttaggaggttcactaaggttgtttgttggggggaggggggggtaaagaaagacatggagacagtgtatccaataaactattga

SEQ ID NO: 321 - DNA, 11697 bp, synthetic construct
  source 1..11697: mol_type = other DNA
atggagttgaatcattttgaacttttatacaaaacaaacaaacaaaaaccaatgggagtggaggaaccggtatacgatgccacggggaggccattgtttggagacccgagtgaggtacacccacaatcaacactgaagctaccacatgatagggggagaggtaacatcaaaacaacactgaagaacctacctaggaaaggcgactgcaggagtggcaaccatctaggcccggttagtgggatatatgtaaagcccggccctgtcttttatcaggactacatgggcccggtctaccatagagcccctctagagttttttaacgaagcgcagttttgcgaggtgaccaaaaggataggtagggtgacaggtagtgacggaaagctttaccatatatatgtgtgcatcgatggttgcatactgctgaagctagccaagagggacgagccaagaaccctgaagtggattagaaatttcaccgactgtccattgtgggttaccagttgctctgatgatggcgcaagtggaagtaaagagaagaagccagataggatcaacaaaggcaaattaaaaatagccccaaaagagcatgagaaggacagcagaactaagccacctgacgctacgattgtagtggaaggagtaaaataccaggttaaaaagaagggtaaagttaaaggaaagagtacccaagacggcctgtaccacaacaagaataaaccaccagaatctaggaagaaattagaaaaagccctattggcatgggcggtaatagcaattatgttgtaccaaccagttgaagccgaaaatataactcaatggaacctgagtgacaacggcactaatggtatccagcatgctatgtaccttagaggggttagcaggagcttgcatgggatctggccggaaaaaatatgcaaaggagtccccacctacctggccacagacacggaactgaaagaaatacagggaatgatggatgccagcgaggggacaaactatacgtgctgtaagttacagagacatgaatggaacaaacatggatggtgtaactggtacaatatagacccctggatacagttgatgaatagaacccaagcaaacttggcagaaggccctccggccaaggagtgcgctgtgacttgcaggtacgataaagatgctgacatcaacgtggtcacccaggccagaaacaggccaacaaccctgaccggttgcaagaaaggaaaaaatttttcttttgcgggtacagttatagagggcccatgtaatttcaatgtttccgtggaggatatcttgtatggggatcatgagtgcggcagtttgcttcaggacacggctctgtacctagtggatggaatgaccaacactatagagaatgccagacagggagcagcgagggtaacatcttggctcgggaggcaactcagcactgccgggaagaggttggagggtagaagcaaaacctggtttggtgcctatgccctatcgccttactgtaatgtaacaagcaaaatagggtacatatggtacactaacaactgcaccccggcttgcctccccaaaaatacaaagataataggccccggaaaatttgacactaacgcggaagacggaaagattctccatgagatggggggtcacctatcagaatttctgctgctctctctggttgttctgtctgacttcgcccctgaaacagccagcgcgttatacctcattttgcactacatgattcctcaatcccatgaagaacctgaaggctgcgacacaaaccagctgaatctaacagtggaactcaggactgaagacgtaataccgtcatcagtctggaatgttggcaaatatgtgtgtgttagaccagactggtggccatatgaaaccaaggtggctttgttatttgaagaggcaggacaggtcgtaaaattagccttacgagcgctgagggatttaaccagggtctggaatagcgcatcaaccacggcattcctcatctgcttgataaaagtattaagaggacagatcgtgcaaggtgtgatatggctgctactagtaactggggcacaaggccggctagcctgcaaggaagattacaggtacgcaatatcatcgaccaatgagatagggctactcggggccgaaggtctcaccaccacctggaaagaatacaaccacgatttgcaactgaatgacgggaccgttaaggccatttgcgtggcaggttcctttaaagtcatagcacttaatgtggtcagtaggaggtatttggcatcattgcataaggaggcttcactcacttccgtgacatttgagctcctgttcgacgggaccaacccatcaactgaggaaatgggagatgacttcgggttcgggctgtgcccgttcgatacgagtcctgttgtcaagggaaagtacaatacaaccttgttgaacggtagtgctttctatcttgtctgcccaatagggtggacgggtgtcatagagtgcacagcagtgagcccaacaactctgagaacagaagtggtaaagaccttcaggagagacaagccctttccgcacagaatggattgtgcgaccaccacagtggaaaatggagatttattctactgtaagttggggggcaactggacatgtgtgaaaggtgaaccagtggtctacacgggggggctagtaaaacaatgcagatggtgtggcttcgacttcaatgagcccgacggactcccgcactaccccataggtaagtgcatcttggtaaatgagacaggttacagaatagtagattcaacggactgtaacagagatggcgttgtaatcagcacagatgggagtcatgagtgcttgatcggtaacacaactgtcaaggtgcatgcatcagatgaaagactgggccctatgccatgcagacccaaagagattgtctctagtgcaggacctgtaaggaaaacttcctgtacattcaactacgcaaaaactttgaagaacaagtactatgagcccagggacagctacttccagcaatatatgcttaagggcgagtatcagtactggtttgacctggacgtgactgaccgccactcagattacttcgcagaatttgtcgtcttggtagtggtagcactgttaggaggaagatatgtcctgtggctaatagtgacctacatagttctaacagaacaactcgccgctggtttaccattgggccagggtgaggtagtgttgatagggaacttaattacccacacagacattgaggtcgtagtatatttcttactactctatttggtcatgagggatgagcctataaagaaatggatactgctgctgttccatgctatgactaacaatccagtcaagaccataacagtggcattgcttatggttagtggggttgccaagggtggaaagatagacggcggttggcagcggctgccagagaccagctttgacatccaactcgcgctgacagttatagtagtcgctgtgatgttactggcaaagagagatccaactactgtccccttggttataacagtggcaaccctgagaacggctaagatgactaatggacttagcacggatatagccatagctacagtgtcaacagcgttgctaacctggacctacattagtgactattatagatacaagacttggctacagtaccttattagcacagtgacaggtatcttcttaataagggtactgaagggaataggtgagttggatttacacactccaaccttgccatcttacagacccctcttcttcattctcgtgtacctcatttccactgcagtggtaacaagatggaatctggacatagccggattgctgttgcagtgtgtcccaacccttttgatggtttttacgatgtgggcagacattctcaccctgatcctcatactgcccacttacgagctaacaaaactatattacctcaaggaagtgaagactggggcagaaaagggctggttatggaagaccaacttcaagagggtaaacgacatatacgaagttgaccaatctggtgaaggggtttaccttttcccgtcaaaacaaaagacaagttcaataacaggtaccatgttgccattgatcaaagccatactcatcagctgcatcagtaataagtggcagttcatatatctattgtacttgatatttgaagtgtcttactacctccacaagaagatcatagatgaaatagcaggagggaccaacttcatctcaagacttgtagccgctttgatcgaagccaattgggcctttgacaacgaagaagttaggggtttaaagaagttcttcctgttgtctagtagggttaaagaactgatcatcaaacacaaagtgaggaatgaagtaatggtccactggtttggtgacgaagaggtttatgggatgccaaagttggttggcttagtcaaggcagcaacattgagtaaaaataaacattgtattttgtgcaccgtctgtgaagacagagagtggagaggagaaacctgcccaaaatgcgggcgttttgggccaccaatgacctgtggtatgaccctagccgactttgaagaaaaacattataagaggatcttttttagagaggatcaatcagaagggccggttagagaggagtacgcagggtatctgcaatatagagccagagggcaattattcctgaggaatctcccggtgctagcaacaaaagtcaagatgctcctggtcggaaatcttgggacggaggtgggagacttggaacaccttggctgggtccttagggggcctgccgtttgcaagaaggttaccgaacatgagaaatgcaccacatccataatggacaaattgactgcttttttcggtgttatgccaaggggcaccacacctagagcccctgtgagattccccacctctctcttaaagataagaagggggttggaaactggctgggcgtacacacaccaaggtggcattagttcagtggaccatgtcacttgtgggaaagacttgctggtatgtgacactatgggccggacaagggtcgtttgccaatcaaataataagatgacagatgagtctgagtatggagttaaaactgactccggatgcccggaaggagctaggtgttatgtgttcaacccagaggcagttaacatatcagggactaaaggagccatggtccacttacaaaaaactggaggagaattcacctgtgtgacagcatcaggaactccggccttctttgatctcaagaacctcaaaggctggtcagggctaccgatatttgaggcatcaagtggaagggtagtcggcagggtcaaggtcgggaagaatgaggactctaaaccaaccaagcttatgagtggaatacaaacagtctccaaaagtaccacagacttgacagaaatggtaaagaaaataacgaccatgaacaggggagaattcagacaaataacccttgctacaggtgccggaaaaaccacggaactccctaggtcagtcatagaagagatagggaggcataagagagtcttggtcttgatccctctgagggcggcagcagagtcagtataccaatatatgagacaaaaacatccaagcatcgcatttaacctgaggataggggagatgaaggaaggggacatggccacagggataacctatgcttcatacggttacttctgtcagatgccacaacctaagttgcgagccgcgatggttgagtactccttcatatttcttgacgagtaccactgtgccaccccagaacaattggccatcatgggaaagatccacagattttcagagaacctgcgggtagtagccatgaccgcaacaccagcaggcacagtaacaaccacagggcagaaacaccctatagaagaattcatagccccagaagtgatgaaaggggaagacttaggctcagagtacttggacattgctggactaaagataccagtagaggagatgaagagcaacatgctggtttttgtgcccactaggaacatggcggtggagacagcaaagaaattgaaagctaagggttacaactcaggctactattatagtggagaggatccatctaacctgagggtggtaacgtcgcagtccccgtacgtggtggtggcaaccaacgcgatagaatcaggtgttactctcccggacttggatgtggttgtcgatacagggcttaagtgtgaaaagagaatacggctgtcacctaagatgcccttcatagtgacgggcctgaagagaatggctgtcacgattggggaacaagcccagagaagggggagagttgggagagtaaagcctgggagatactacaggagtcaagaaactcccgttggttctaaagattaccattatgatctactgcaagcacagaggtacggtattgaagatgggataaacatcaccaaatcctttagagagatgaactatgattggagcctttatgaggaggacagtctgatgattacacaattggaaatcctcaataatttgttgatatcagaagaactaccgatggcagtaaaaaatataatggccaggactgaccacccagaaccaattcagctggcgtacaacagctacgaaacacaagtgccagtgctattcccaaaaataaagaatggagaggtgactgatagttacgataactataccttcctcaacgcaagaaaattgggggatgatgtacccccttacgtgtatgccacagaggatgaggacttagcggtagagctactgggcttagactggccagaccctggaaaccaaggaaccgtagaggctggcagagcactaaaacaagtagttggtctatcaacagctgagaatgccctgttagtagccttattcggctatgtaggatatcaggcactttcaaagaggcatataccagtagtcacagatatatattcaattgaagatcacaggttggaagacaccacacacctacagtacgccccaaatgctatcaagacggaggggaaggagacagaattgaaggagctagcccagggggatgtgcagagatgtgtggaagctatgaccaattatgcaagagagggtatccaattcatgaagtctcaggcactgaaggtgaaagaaacccccacttacaaagagacaatgaacactgtgactgactatgtaaagaaattcatggaggcgctggcagacagtaaagaagacatcttaagatatgggttgtgggggacgcacacagccttatataagagcatcagtgccaggcttgggagtgagactgcgttcgctaccctggtcgtgaagtggctggcatttgggggggaatcaatagcagaccatgtcaaacaagcggccacagacttggtcgtctactatatcatcaacagacctcagttcccaggagacacagagacacaacaggaaggaaggaaatttgtggccagcctactggtctcagctctagctacttacacatacaaaagctggaattacaataatctgtccaagatagttgaaccggctttggccactctgccctatgccgccacagctctcaaactattcgcccccactcgattggagagcgttgtcatattgagtaccgcaatctacaagacctacctatcaatcaggcgcggaaaaagcgatggtttgctaggcacaggggttagtgcggctatggagatcatgtcacaaaatccagtatccgtgggcatagcagtcatgctaggggtaggggccgtggcagcccacaatgcaatcgaggccagtgagcagaagagaacactactcatgaaagtttttgtaaagaacttcttggaccaagcagccactgatgaattagtcaaggagagtcctgagaaaataataatggctttgtttgaagcagtgcagacagtcggtaaccctcttagactagtataccacctttatggagttttctataaggggtgggaggcaaaagagttggcccaaaggacagccggtaggaaccttttcactttgataatgttcgaggctgtggaactactgggagtagatagtgaaggaaagatccgccagctatcaagtaattacatactagagctcctgtataagttccgtgacagtatcaagtctagcgtgagggagatggcaatcagctgggcccctgcccctttcagctgtgattggacaccgacggatgacagaatagggctcccccaagacaatttcctccaagtggagacgaaatgcccctgtggttacaagatgaaggcagttaagaattgtgctggagagctgagactcttggaggaggaaggctcatttctctgcagaaataaattcgggagaggttcacggaactacagggtgacaaaatactatgatgacaatctatcagaaataaagccagtgataagaatggaagggcatgtggaactctactacaagggagccaccatcaaactggacttcaacaacagtaaaacaatactggcaaccgataaatgggagattgatcactccactctggtcagggtgctcaagaggcacacaggggctggatatcatggggcatacctgggcgagaaaccgaactacaaacatctgatagagagggactgtgcaaccatcaccaaagataaggtttgttttctcaaaatgaagagagggtgtgcatttacttatgacttatcccttcacaaccttacccgactgattgaattggtacacaagaataacttggaagacaaagagattcctgctgttacggttacaacctggctggcttacacgtttgtaaatgaagatatagggaccataaaaccagccttcggggagaaagtaacaccggagatgcaggaggaaataaccttgcagcctgctgtagtggtggatacaactgacgtgaccgtgactgtggtaggggaagcccctactatgactacaggggagactccgacagcgttcaccagctcaggttcagacccgaaaggccaacaagttttaaaactgggggtaggtgaaggccaataccccgggactaatccacagagggcaagcctgcacgaagccatacaaggtgcagatgagagaccctcggtgctgatattagggtctgataaagccacctctaatagagtgaaaactgcaaagaatgtaaaggtatacagaggcagggacccactagaagtgagagatatgatgaggaggggaaagatcctggtcatagccctgtctagggttgataatgctctattgaaatttgttgactacaaaggcacctttctaactagagagaccctagaggcattaagtttgggtaggcctaaaaagaaaaacataaccaaggcagaagcgcagtggttgctgtgcctcgaagaccaaatggaagagctacccgattggttcgcagccggggaacccatttttctagaggctaacattaaacatgacaggtaccatctggtgggggatatagctaatatcaaggaaaaagccaaacagttgggagctacagactccacaaagatatctaaggaggttggtgcaaaagtgtattctatgaaactgagtaattgggtgatgcaagaagaaaataaacagggcaacctgacccccttgtttgaagagctcctgcaacagtgtccacccggaggccagaacaaaactgcacatatggtctctgcttaccaactagctcaagggaactggatgccaaccagctgccatgtttttatggggaccatatctgccaggaggaccaagacccatccatatgaagcatacgtcaagttaagggagttggtagaggaacacaagatgaaaacattgtgtcctggatcaagcctgggtaagcacaacgaatggataattggtaaaatcaaataccagggaaacctgaggaccaaacacatgttgaaccccggcaaggtggcagagcaactgtgcagagagggacacagacacaatgtgtataacaagacaataggctcagtaatgacagctactggtatcaggttggagaagttgcccgtggttagggcccagacagacacaaccaacttccaccaagcaataagggataagatagacaaggaagagaacctacaaaccccgggtttacataagaaactaatggaagttttcaatgcattgaaacgacccgagttagagtcctcctacgatgccgtggaatgggaggaactggagagaggaataaacaggaagggtgctgctggtttctttgaacgcaaaaatataggggaaatattggattcagagaaaaacaaagtcgaagagattattgacaatctgaaaaaaggcagaaacatcaaatactatgaaaccgcgatcccaaagaatgagaagagggacgtcaatgatgactggactgctggtgacttcgtggaagagaagaaacccagagtcatacaataccctgaagcaaaaacaaggctggccatcaccaaggtgatgtataagtgggtgaagcagaagccagtagttatacccgggtatgaagggaagacacctctattccaaatttttgacaaagtaaagaaggaatgggatcaattccaaaatccagtggcagtgagttttgacactaaggcgtgggacacccaggtaaccacaaaagatttggagttgataaaggacatacaaaagtactatttcaagaagaaatggcataaatttattgacaccctgaccatgcacatgtcagaagtacccgtaatcagtgctgatggggaagtatacataaggaaagggcaaagaggcagtggacaacctgacacaagcgcaggcaatagcatgctaaatgtgttaacaatgatttacgccttctgcgaggccacgggagtaccctacaagagcttcgacagggtggcaaaaattcatgtgtgtggggatgatggtttcctgatcacagaaagagctctcggtgagaaattcgcgagtaagggagtccagatcctatatgaagctgggaagccccagaagatcactgaaggggacaagatgaaagtggcctaccaatttgatgatattgagttttgctcccatacaccaatacaagtaaggtggtcagataacacttctagttacatgccggggagaaatacaaccacaatcctggctaaaatggccacaaggttagattccagtggtgagaggggtaccatagcatatgagaaagcagtagcattcagcttcctgctgatgtactcctggaacccactaatcagaaggatctgcttactggtgctatcaactgaactgcaagtgaaaccagggaagtcaaccacttactactatgaaggggacccgatatctgcctacaaggaagtcatcggccacaatctttttgatcttaagagaacaagcttcgagaagctggccaagttaaatctcagcatgtcagtactcggagcctggactagacacaccagtaaaagactactacaagactgtgtcaatgtgggtgttaaagagggcaactggctagttaatgcagatagactagtaagtagcaagactggaaataggtacatacccggagagggccacaccctgcaagggagacattatgaagaactggtgttggcaagaaaacagatcaacaactttcaagggaccgacaggtacaatctaggcccaatagtcaatatggtgttaaggaggctgagagtcatgatgatgaccctgatagggagaggggtatga

SEQ ID NO: 322 - DNA, 1119 bp, synthetic construct
  source 1..1119: mol_type = other DNA
cggctagcctgcaaggaagattacaggtacgcaatatcatcgaccaatgagatagggctactcggggccgaaggtctcaccaccacctggaaagaatacaaccacgatttgcaactgaatgacgggaccgttaaggccatttgcgtggcaggttcctttaaagtcatagcacttaatgtggtcagtaggaggtatttggcatcattgcataaggaggcttcactcacttccgtgacatttgagctcctgttcgacgggaccaacccatcaactgaggaaatgggagatgacttcgggttcgggctgtgcccgttcgatacgagtcctgttgtcaagggaaagtacaatacaaccttgttgaacggtagtgctttctatcttgtctgcccaatagggtggacgggtgtcatagagtgcacagcagtgagcccaacaactctgagaacagaagtggtaaagaccttcaggagagacaagccctttccgcacagaatggattgtgcgaccaccacagtggaaaatggagatttattctactgtaagttggggggcaactggacatgtgtgaaaggtgaaccagtggtctacacgggggggctagtaaaacaatgcagatggtgtggcttcgacttcaatgagcccgacggactcccgcactaccccataggtaagtgcatcttggtaaatgagacaggttacagaatagtagattcaacggactgtaacagagatggcgttgtaatcagcacagatgggagtcatgagtgcttgatcggtaacacaactgtcaaggtgcatgcatcagatgaaagactgggccctatgccatgcagacccaaagagattgtctctagtgcaggacctgtaaggaaaacttcctgtacattcaactacgcaaaaactttgaagaacaagtactatgagcccagggacagctacttccagcaatatatgcttaagggcgagtatcagtactggtttgacctggacgtgactgaccgccactcagattacttcgcagaatttgtcgtcttggtagtggtagcactgttaggaggaagatatgtcctgtggctaatagtgacctacatagttctaacagaacaactcgccgctggt

SEQ ID NO: 323 - DNA, 6999 bp, synthetic construct
  source 1..6999: mol_type = other DNA
atgaacacagctggttgttttatcgctttgttgtacaccatcagagagataaaaacacgactgttttcaaggacacaggaagaaatggaattcacactttacaacggtgagaagaagatcttctactccaggcccaacaaccacgacaactgttggctgaacgccatccttcagctgttcaggtacgtcgatgaacctttcttcgactgggtatatgaatcacctgaaaacctcacccttgaggcgatcagacaactggagaacattactggtcttgagctgcacgagggtggtccgcccgccctcgtcatttggaacatcaaacacttgctccacaccgggatcggcaccgcctcgcgacccagcgaggtgtgcatggtggacggtacggacatgtgcctggctgacttccacgctggcatcttcctgaaaggacaggaacacgccgtgtttgcctgcgtcacctccaacgggtggtacgcgatcgacgacgaagaattctacccctggacgccagatccgtccgacgtgctggtctttgtcccgtacgatcaagaaccacttaatggggaatggaaagcaagggttcagagacggctcaagggagccggacaatccagtccggctactgggtcacagaaccaatcaggcaacaccgggagtatcatcaacaattactacatgcagcagtaccagaactccatggacacccaacttggtgacaatgctatcagcggaggctccaacgagggatccacagacacaacctccacccacacaaccaacactcagaacaatgactggttttcaaagttggccagctctgccttcagcggtctcttcggcgccctcctcgccgataagaaaaccgaggagaccactcttctcgaggaccgcatcctcaccacccgaaacggacacaccacctcgacaacccagtcgagtgttggcataacgcacgggtacgcaacagctgaggactttgtgaacgggccaaacacctctggtcttgagaccagagttgtccaggcggaacggttctttaaaacccacctgttcgactgggtcaccagtgatccgttcggacggtactacttgttggagctcccgactgaccacaaaggtgtctacggcagcctgaccgactcatacgcctacatgagaaacggttgggatgttgaagtcaccgctgtggggaatcagttcaacggaggctgcctactggtggccatggtgcctgaactttgttccatcgagcggagagagttgttccagcttacgctcttcccccatcagttcatcaacccccggacgaacatgacagcccacatcaaggtgccctttgttggcgtcaaccgttacgatcagtacaaggtacacaagccgtggacccttgtggttatggtcgtagccccactgactgtcaacaccgaaggcgctccgcaaatcaaggtgtatgccaacatcgcgcccaccaacgtgcacgtcgcgggtgagttcccttccaaagaggggattttccctgtggcatgtagcgacggttatggcggcttggtgacaactgacccaaagacggctgaccccgtttacggcaaagtgttcaaccccccccgcaacatgttgccggggcggttcaccaacctcctggacgtggctgaggcttgccccacgtttctgcacttcgatggtgacgtaccgtatgtgaccactaagacggattcggacagggtgctcgcacaatttgacttgtctttggcagcaaaacacatgtcaaacaccttccttgcaggtcttgcccagtactacacgcagtacagcggcaccgtcaacctgcacttcatgttcgcaggtcccactgacgcgaaagcgcgttacatgattgcgtatgcccctccgggcatggagccgcccaaaacacctgaggctgctgctcactgcattcacgcagagtgggacacgggtctgaactcaatgtttaccttttccatcccctacctctcggcggctgattacgcgtacaccgcgtctgacgctgctgagaccacaaatgttcagggatgggtctgcttatttcaaataacacacgggaaagctgagggtgacgctcttgtcgtgatggccagtgctggcaaagactttgagctgcgcctgcctgtggacgctcggcaacagaccacttcgacaggcgagtcggctgaccccgtgactgccaccgttgagaattacggcggcgagacacaggtccagaggcgccaccacacagacgtctcattcatattggacagatttgtgaaagtcacaccaaaagactcaataaatgtattggacctgatgcagaccccctcccacaccctagtaggggcgctcctccgcactgccacttactatttcgctgatctagaggtggcagtgaaacacgagggggaccttacctgggtgccaaatggagcacctgaagcagccttggacaacaccaccaacccaacggcgtaccataaggcgccgcttacccggcttgcattgccctacacggcaccacaccgtgttttggccaccgtttacaacgggaactgcaaatacgccgggggctcactgcccaacgtgagaggcgatctccaagtgctggctccgaaggcagcgaggccgctgcctacttctttcaactacggtgccatcaaagccactcgggtgacagaactgctgtaccgcatgaagagggccgaggcgtactgtcctcggcccctcttggctgttcacccgagtgcggccagacacaaacagaaaatagtggcgcctgtaaagcagtccttgaactttgatctgctcaagttggcaggggacgtggagtccaaccctgggcccttcttcttctctgacgtcaggtcaaacttcaccaaactggtggaaaccatcaaccagatgcaagaggacatgtcaacaaaacacggacccgactttaaccggttggtatccgcgtttgaggaattggccactggggtgaaagccatcaggaccggcctcgacgaggccaaaccctggtacaaactcatcaagctcctgagccgcttgtcatccatggccgctgtagcagcacggtccaaggacccagtccttgtggccatcatgctggctgacaccggtcttgagattctggacagcacatttgtcgtgaagaaaatctccgaccccctctccagtctctttcacgtgccggcccccgtcttcagtttcggagctccgattctgctagccgggttggtcaaggtcgcttcgagcttcttccggtccacacccgaggatctcgagagagcagagaaacagctcaaagcacgtgacatcaatgacatcttcgccattctcaagaacggcgagtggctggtcaagttgatcctagccatccgcgactggattaaagcatggatcgcctcagaagagaagtttgtcaccatgacagacttggtgcctggcatccttgaaaagcagcgggacctcaacgacccggccaagtacaaggaagccaaggaatggctcgacaacgcgcgccaaacgtgtttgaagagcgggaacgtccacattgccaacctgtgcaaagtggtcgccccagcgccgagcaagtcgagacctgaacccgtggtcgtgtgcctccgcggcaaatccggtcagggtaagagtttccttgcgaacgtgctggcacaagccatctctacccactttaccggcaggactgactcagtttggtactgtccgccagaccctgaccacttcgacggttacaaccagcagaccgttgttgtgatggatgatttgggccagaatcccgacggcaaggacttcaagtacttcgctcagatggtctcgaccacggggttcatcccgcccatggcttcacttgaggacaaaggcaagcctttcaacagcaaagtcatcattgccaccaccaacctgtactcgggcttcaccccgagaaccatggtgtgccccgatgctctgaaccgaaggtttcactttgacattgacgtgagtgccaaggacgggtacaaaattaacaacaaattggacataatcaaagctctcgaggacacccacaccaaccctgtggcaatgttccaatacgactgtgcccttctcaacggcatggccgttgaaatgaagagaatgcaacaagacatgttcaaaccccagccgcctttgcagaacatataccaacttgtgcaagaggtgattgaccgggtcgagctccacgagaaagtgtcgagccacccgattttcaagcagatctcaattccttcccaaaagtcagtgctgtatttcctcattgagaaaggccaacacgaagcagcaattgaattctttgaggggatggtccatgactccatcaaggaagagctccgacccctcatccaacagacatcatttgtcaagcgcgccttcaagcgcctgaaggaaaactttgagattgttgccctatgtttgactctcatggcaaacatagtgatcatgatccgcgaaactcgcaagagacagcagatggtggatgatgcagtgaatgagtacatcgagaaagcaaacatcaccacagatgacaagactcttgacgaggcggaaaagaaccctctagagactagcggtgccagcactgttggtttcagagagagaactctcccgggacacaaggtgggtgatgacgtgaactccgagcccgcccaccccggggatgagcaaccacaagctgaaggaccctacgccggaccactcgaccgtcagagacctctgaaagtgagagccaagctgccacagcaggagggaccttacgccggtccgatggagagacagaaaccactgaaagtgaaagcgaaagccccggtcgtgaaggaaggaccttacgagggaccggtgaagaagcctgtcgctttgaaagtgaaagctaggaacttgatcgtcaccgagagtggtgcccccccgaccgacttgcaaaagatggtcatgggtaacaccaagcccgttgagctcatactcgacgggaagacagtagccatctgctgtgctactggagtatttggcactgcctacctcgtgcctcgtcatcttttcgctgagaagtacgacaagatcatgttggacggtagaaccatgatagacagtgactacagagtgtttgagtttgagatcaaagtaaaaggacaggacatgctctcagacgctgcgctcatggtgttgcaccgtgggaaccgcgtgagagacatcacgaaacactttcgtgacacagcaagaatgaagaaaggtacccccgtcgttggtgtgatcaacaacgctgacgtcgggagactgattttctcaggtgaggccctcacctacaaggacattgtagtgtgcatggatggagacaccatgccgggcctatttgcctacaaagccgccaccaaggctggctactgcgggggagccgtccttgctaaggatggagctgacacattcatcgttggcactcactctgcaggtggcaatggagttgggtactgctcatgcgtatctagatccatgctccaaaaaatgaaggcacacatcgaccctgaaccacaccacgaggggttgatcgtagacaccagagatgtggaagagcgcgtgcacgtcatgcgcaaaaccaagcttgcacctaccgtggcacacggtgtgttcaaccctgagtacggccccgctgccttgtccaacaaggacccgcggctgaatgagggagttgtcctcgatgaggtcatcttctccaaacacaagggggacacaaagatgtcaccggaagacaaagcgctgttccgccgctgcgctgccgactacgcgtcgcgtcttcacagtgtgctgggtacagcaaatgccccattgagcatctacgaggccattaaaggcgttgacggactcgacgccatggaaccagacacagcgcctggccttccctgggcactccaggggaaacgccgcggcgcgctgattgacttcgagaacggcactgtcggacccgaagtccaggctgccctggagctcatggagaaaagagaatacaagtttgcctgtcagaccttcctgaaggacgaaattcgcccgatggaaaaagtacgtgccggcaagacgcgcatcgtcgatgttttgcctgttgaacacattctttacaccaggatgatgattggcagattttgtgctcaaatgcactcaaacaacggaccgcaaattggatcagcggtcggttgtaatcctgatgttgattggcaaagatttggcacacacttcgcccaatacagaaacgtgtgggatgtggactattcggcctttgatgctaaccactgtagtgatgcaatgagcatcatgtttgaggaggtgtttcgcacagactttggtttccacccgaatgctgagtggattctgaagaccctcgtgaacacggaacacgcctatgagaacaaacgcattacagttgaaggtggaatgccgtccggctgttccgcaaccagcatcatcaacacaattctgaacaacatctacgtgctctacgcgctgcgtagacactatgagggagttgagctggacacttacaccatgatctcctacggagacgacatcgtggttgctagtgattatgacttggactttgaggctctcaagccccactttaaatctcttggtcaaaccattactccagctgacaaaagcgacaaaggttttgttcttggtcactccattaccgatgtcactttcctcaaaagacacttccacatggattatggaactgggttttacaaacctgtgatggcttcgaagaccctcgaggctatcctctcctttgcacgccgtgggaccatacaggagaagttgatctccgtggcagggctcgccgtccactctggacctgacgagtaccggcgtctcttcgagcccttccagggtctctttgagattccaagctacagatcactttacctgcgttgggtgaacgccgtgtgcggtggcgcataa

SEQ ID NO: 324 - DNA, 1097 bp, synthetic construct
  source 1..1097: mol_type = other DNA
ttgaaagggggcgctagggtctcacccctaacatgccaacgacagctcctgcgtcgcactccacacttacgtctgtgcgcgcgcgggaaccgatggactttcgttcacccacctgcagctggactcacggcaccgcgtggccattttagctggactgagcggacgaacgtcgcttgcgcacctcgcgtgatcgactagtactctcaacactccgcctattcggtcgttagcgctgtcctgggcactcctgctgggggccgttcgacgctctacggtttccccccccgcgacaaactacggtgatggggccgcttcgtgcgagccgatcgcctggtgtgtttcggttgtcactcgaaacccacctttcacccccccccccccccccctaagtactaccgtcgctcccgacgttaaagggaggtaaccacaagatttgcgccttcttgtccgaagttagagggctgtaaccgcaaactttgaaccgcctttcccagcgttaacgggatgtaatcacaagatggaccttcatccggaagtaaaacggcaacttacacagttttgcccgttttcatgagaaatgggacgtcagcgcacgaaacgcgcagtcgcttgaggaggacttgtacaaacacgtctcacacaggtacccacaaccgacacaaaacgtgcaacttgaaatcccgcctggtctttccaggtctagaggggtgacactttgtactgtgattgactccacgctcggcccactggcgagtgttagtagtagtactgttgcttcgtagcggagcatggtggccgtgggactccctccttggtaacaaggacccacggggccgaaagccacgtctcaggacccaccatgtgtgcaaccccagcacggcaactttaccacgaaaaccattttaaggtgacactgaaactggtactcaaccactggtgacaggctaaggatgcccttcaggtaccccgaggtaacacgcgacactcaggatctgagaaggggattggggcttctgtaaaagcgcccagtttaaaaagcttctatgcctgaataggcgaccggaggccggcgcctttccttaactattactgcttac

SEQ ID NO: 325 - DNA, 759 bp, synthetic construct
  source 1..759: mol_type = other DNA
atgagtcttctaaccgaggtcgaaacgtatgttctctctattgttccgtcaggccccctcaaagccgagatagcgcagagacttgaagatgtttttgcagggaaaaacaccgatctagaggcactcatggaatggctaaagacaagaccaatcctgtcacctctgactaaggggattttaggatttgtattcacgctcaccgtgcccagtgagcgaggactgcagcgtagacgctttgtccagaatgccctcaatgggaatggtgacccgaacaacatggacaaagcagtcaaactgtacagaaaacttaaaagggaaataacatttcacggggctaaagaagtagcgctcagttactctgctggtgcacttgccagttgtatgggcctcatttacaacagaatggggactgtcaccactgaggtggcatttggcctagtatgcgcaacctgtgaacagattgccgattcccagcatcgatctcatagacaaatggagacaacaaccaatccactaattaggcatgagaacagaatggtattagccagcacgacagctaaagccatggaacaaatggctggatcaagtgaacaagcagcagaggctatggaaattgccagccaagctaggcaaatggtacaggcaatgagaacaattgggactcatcctagttccagcgctggtctaaaagatgatcttcttgaaaatttacaggcctatcagaaacgaatgggagtgcagatgcaacggttcaagtga

SEQ ID NO: 326 - DNA, 1701 bp, synthetic construct
  source 1..1701: mol_type = other DNA
atgaaggcaatactagtagtcttgctatatgcatttacaaccgcaaatgcagacacattatgtataggttaccatgcaaataattcaactgacactgttgacacagtactagaaaagaatgtaacagtaacacactctgttaaccttttagaagacagacataacgggaaactatgtaaactaaaaggggtagccccattgcatttgggtaaatgtaacattgctggatggctactgggaaacccagagtgtgaattactattcacagcaagctcatggtcttacattgtggaaacttctaattcagacaatgggacatgttacccaggagatttcatcaattatgaagagctaagagagcagttgagctcagtgtcatcatttgaaagatttgagatattccccaaggcaagttcatggcccaatcatgaaacgaacagaggtgtgacatcagcatgtccttattctggagcaaacagcttctacagaaacttaatatggctggtaaaaaaaggaaattcatacccaaaactcagcaaatcctatattaacaataaggggaaggaagttcttgttctatggggcattcaccatccacctaccagtactgaccaacaaagtctctaccagaatgcagatgcctatgtttttgtggggtcatcaaaatacaacaagaaattcatgccagaaatagcaacaagacccaaagtgagaggtcaagcagggagaatgaactattactggacactaattgagcctggagacacaataacattcgaagcaactggaaatctagtggcaccaagatatgccttcgcaatggaaagaggctctggatctggtattataatttcagatacaccagtctacgattgtaatacggcttgtcaaacacccaaaggtgctataaacaccagtcttccatttcaaaatatacatccagtcacaattggagaatgtccaaaatatgtcaaaagcacaaaattgagaatggctacaggattaaggaacatcccgtctattcaatctagaggcctgtttggagccattgctggctttattgagggggggtggacaggaatgatagatggatggtatggttatcatcaccaaaatgagcaggggtcaggatatgcaccagaccgaaagagcacacagaatgccatagacgggatcactaacaaagtaaactctgttattgaaaagatgaacacacaattcacagcagtgggtaaagaattcaatcacttggagaaaagaatagagaatttaaacaaaaaggttgatgatggttttctgaatgtttggacttacaatgccgaactgttggttctattggaaaatgaaagaactttggattatcacgattcaaatgtgaagaacctatatgagaaagtaagaagccaactaaaaaacaatgccaaggaaattggaaatggctgctttgaattttaccacaaatgtgatgacacgtgcatggagagcgtcaaaaatgggacttatgattatccaaaatactcagaagaagcaagactaaacagagaagagatagatggggtaaagctggaatcaacaaggatttaccagattttggcgatctattcaactatcgccagttcattggtactgttagtctccctgggggcaatcagtttctggatgtgctccaatgggtctttacagtgcagaatatgtatttaa

SEQ ID NO: 327 - DNA, 1701 bp, synthetic construct
  source 1..1701: mol_type = other DNA
atgaagactatcattgctttgagctacattttatgcctggttttcgctcaaaaacctcccggaaatgacaacagcacggcaacgctgtgcctggggcaccatgcagtgccaaacggaacgctagtgaaaacaatcacgaatgaccaaattgaagtaactaatgctactgagctggttcagagttcctcaacaggtaaaatatgcgacagtcctcaccaaatccttgatggagaaaactgcacactaatagatgctctattgggagaccctcattgtgatggcttccaaaataaggaatgggacctttttgttgaacgcagcaaagcctacagcaactgttacccttatgatgtgccggattatgtctcccttaggtcactagttgcctcatcaggcatgctggagtttaacaatgaaagcttcaattggactggagtcgctcagaatggaacaagctctgcttgcaaaaggagatctgataaaagtttctttagtagattgaattggttgcaccaattaaaatacaaatatccagcactgaacgtgaccatgccaaacaatgaaaaatttgacaaattgtacatttggggggttcaccacccgggtacagacagtgaccaaatcagcctatatgctcaagcatcagggagagtcacagtctctaccaaaagaagccaacaaactgtaatcccgaatatcggatctagaccctgggtaaggggtgtctccagcagaataagcatctattggacaatagtaaaaccgggagacatacttttgattaacagcacagggaatctaattgctcctcggggttacttcaaaatacgaagtgggaaaagctcaataatgaggtcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcggaatgtgccagagaaacaaactagaggcatattcggtgcaatcgcgggcttcatagaaaatggttgggagggaatgatggacggttggtacggtttcaggcatcaaaattctgagggcacagggcaagcagcagatcttaaaagcactcaagcagcaatcaaccaaatcaacgggaaactgaataggttaatcgagaaaacgaacgagaaattccatcaaattgaaaaagaattctcagaagtagaagggagaattcaggacctcgagaaatatgttgaggacactaaaatagatctctggtcgtacaatgcggagcttcttgttgccctagagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagggtcaatcagaaatggaacttatgaccatgatgtatacagagacgaagcattgaacaaccggttccagatcaaaggtgttgagctgaagtcaggatacaaagattggatcctatggatttcctttgccatatcatgttttttgctttgtattgttttgctggggttcatcatgtgggcctgccaaaaaggcaacattaggtgcaacatttgcatttga

SEQ ID NO: 328 - DNA, 6573 bp, synthetic construct
  source 1..6573: mol_type = other DNA
atgaataaaatatttagagttatttggagtcatgctcaacaggcttgggtggttgtatctgagttagtaaagtctcataccaaaacatccgcttacacggataaaagagctcaagtatgcacctcagattattttttagataaacagcaagataaatttaaattaagtcttttaagtctagtattactaggtatattttttagttcagtaggttcagctgcatatcttcaagatggtgctaatgagggatcgaatataggaactgacgacggtactattggtattggtcaagagagtagggcttcgtatggtgctgttgctatcggtcagaaggcaaaagctgaagctaggcataatattgcgataggttatggagcagattcaggaacacaagtaaactctcttgcgataggctaccgcgctacagtaagtgggactggagcaattgccttaggcaaacaagccaattcgacaaatagtcaaacaattgccatcgggagtgattcaaaagcgagcggagatgaatctattgccttaggcggacaagccaattcgacaaataatcaaacaattgccatcgggagtcagtcaaaagcgagcggagaacaatctattgtcttaggcacaggggccagtgtgacaggtactcaaacaattgtaataggcgcacgtgctagtgcaagtgggcaccaatctgttgctatcggggcaaatacccaagcgcaggggtatggctccatatcaatcggtggagatgatttagctacaacgaaatatcaagatgatgctcaagactattccagcacaacaactgcggcaggtcacgcctctgttgctattgggggtcgatctaaggctcagggcgatggatctattgttgtaggtcctttagcatatgcaaggtatgctgaaggcattgctatcggtgcgagaagtaagtctaacaatgagtacggtattgcggttggtagtggtgcatttgctggaaagcactccattgctgttggtaaaagtgctactgcaagtcaaacaggagcttccgcattcggtgaaagcgcacgagcaatcggacagtatacaacagcattggggagttatgcggaagcagaaacgcaagatggcgttgctctaggttatcgatccaaaactagtcgtcggtcagggcaaagaggttggacaccagagaatacaaattattctattaacggaagtacattatctgctacgcatgcagcggtagcagtaggtgatggcaccaccgtcactcgtcaaattaccagtgttgctgcagggacagcagatactgacgcagcaaacgtggcgcaattaaaagcactgactttaaagatcagtggcgatagaaatacgcagggtcacacaacattttataacgaaacgctctcaattgtaggggctgatggtattagtacggcagttgagcagggaaatggcaattcaaaaattaccattactggctcaaaaacgtattttcatactaactataacgaacagtctcaaggtcggggagatcctacgactaatttcggcacaattactgacaaagctggagctacaggaacctatgcaataacagcgggggtaaatgcctctgcggctggaaattatggtattgcgatggggtataagagtaatgctagtgcttatgctgttgctcttggtagtgaatcgaaaggtgctggggcagattccattgcgataggtaatcttgcgagaacaacaggggtagactctgttgttgtgggtgcccatatcaatgtgacaggtcaaaaatcagtggcagttggacgccaagcaaatgctagagattattctactgccttgggttatatggcctctgccagtggtacgtactctgttgctgtgggtgaaaatgccacgataaatgtaaatgctgctagagcaaccgcacttggtcataataccgttgtcaccgtgggtggcggtgtggcattaggttatggatctaatgcaaatatagctggcggtgtagaggggttaaaacaagctcattctgtcacaacgggaacaagcactgaagctaacggctttaaatccacacaaaaggttgatggtaataatattggtgcagtttctgtgggtggtacaaccatcaaacgccaaatcgtcaatgtggcggctggtacacaagataccgatgcggtaaacgtggcacagcttaaatctttgacgatgaaaattggtggtaataccaatgctaatacacagccaaaagtggggttgtgggatggtattcttaaagtgctaggcacaaatggcgaaatcaagaccaatgcgtctggctcaaccattacaatatcactagacgatacgattaaaaataaattagctgatgccagagcaggaagtttgatattcaagggcgaaaaaacagataacggtacaacaaatgatgtttcgggtcaaaaatggaaggccaacgaagataagaccgttaccattacaagtaacgaaacataccaaaacggtggtgttcgatacaaaggcgataacattgaaatttatcgtagaaatctagaattccatgtgttgatgaaagagacaccaacattcagcagcgttcaatatggcgataatggacctaagattaccagcaccactgacggtaatctaaaagtaacaggtacagacggcacttcctcagttaagatcaccaatttagcacgaggtacacaaaatagcgatgcggtgaactacatgcaattttcaaatgctggttggaaacttgcaattgctccaggaacggggggtcaagcaactccacctggggcacatcttatcaaaataaacgataccgtaacctttaccgctggaaataatattaaattagaacaagcgggcggaaatattacgatttctacgattggtaagttaattaaaacgactgaaagcctagaaaatggcgatctaaaaattatctatacagataattcgcataacattatcaagaaaggtgaaaaaggagatcgtggcgagcgaggtctaagaggtgaaacaggccctgcgggtccgattggtccagtgggtccagcaggggctaggggtgagcgaggccctgcaggtgtagctggtcctaagggtgagaaaggtgatccaggaccagagggccctcaaggtcctagaggtgagcaaggtctaagaggtgaacgaggcctagcgggtcctagaggcgaaactggtccgaggggtccagtgggaccacaaggagcgcaaggaatgccgggtgctcagggacagaagggtgaccgaggagaaactggccctgcgggtcctagaggcgaagctggtcctgctggagcaacaggaccacaaggtgaaaaaggagatcaaggtccgatgggtccggcaggcccagcgggagagcgaggcgagcggggtcctaaaggagatagaggtccaaaaggtgatacaggtgagagaggtgcaactggccctgcgggtccgatgggtccagcaggcccagcgggagagcgaggagaaactggacctgcaggtgtacctggtcctaggggtccagagggtcctagaggcgaaactggtttaacaggtccgaggggtccagtgggaccacaaggaccgcaaggaacaccgggtactccgggacagaagggggataaaggcgatccaggacaagcgggtccagcaggacctaggggtccagtgggtccgaaaggtgaaacaggagctcaaggtccgataggtccggcaggcccagcgggagagcgaggcgagcagggtcctagaggcgaagctggtccaacaggtccaacaggtccgaggggtccagtgggaccacaaggaccgcagggaacagcgggtgctcagggacctaagggtgagcgaggccctgcaggtgcagctggccctgcgggtccagtaggtccagcgggaccacaaggtgaaaaaggagatcaaggtccgatgggtccggcaggcccagcgggtgctcagggcatacaaggtccaaaaggtgatagaggcgaacaagggcttccaggggtagcaggtcctaagggcgatagaggcgaagctggtccagtgggtccagcaggaccagcgggagagcgaggcgagcagggtcctaggggggaacaaggtgcaactggccctgcgggtccaacggggcctaggggtgaaccgggtccaacgggaccacaaggaccgcaaggaacaccgggtactccgggacagaagggggataaaggcgatccaggacaagcgggtccagcaggacctaggggtccagtgggtccgaaaggtgaaacaggagctcaaggtccgataggtccggcaggcccagcgggagagcgaggcgagcagggtcctagaggcgaagctggtccaacaggtccaacaggtccgaggggtccagtgggaccacaaggaccgcagggaacagcgggtgctcagggacctaagggtgagcgaggccctgcaggtgcagctggccctgcgggtccagtaggtccagcgggaccacaaggtgaaaaaggagatcaaggtccgatgggtccggcaggcccagcgggtgctcagggcatacaaggtccaaaaggtgatagaggcgaacaagggcttccaggggtagcaggtcctaagggcgatagaggcgaagctggtccagtgggtccagtaggaccacgaggaccacagggaacagcgggtgctcagggacctaagggtgagcgaggccctgcaggtgaaactggacctaagggtgagaaaggtgatccaggaccaaaaggcgaaactggtccaacaggtccagtgggtccagcaggaccagcgggagagcgaggcgagcagggtcctaggggggaacaaggtgcaactggccctgcgggtccaacggggcctaggggtgaaccgggtccaacgggaccacaaggaccgcaaggaacaccgggtactccgggacagaagggggataaaggcgatccaggacaagcgggtccagcaggaccacgaggccctgcaggtgcagctggacctgcgggcccagcagggcctaggggtgaccgaggagaaactggtcctgcgggtccaacaggggctaagggtgaacagggtcaaaaaggagatacaggtccaatggggcctgctggtccaaaaggtgatgcaggtcctagaggcgaagctggtcctgctggagcaacaggaccacaaggtccaaaaggagataatggagctacaggtcctaggggagagaaaggtgaacctggccctgcgggtccgattggtccagtgggtccagcaggggctgctggtccagcaggcccagcgggagagcgaggccctacgggtcctaaaggagatgcaggtccaaaaggagatacaggtcagaaaggagaaactggccctgcgggtccagcaggggctaagggtgaaccgggtcctagaggtgagcaaggtattcaaggacctacgggtccaacgggaccacaaggaccgcagggaacagcgggtattcagggacctaagggtgagcgaggaaatgtgagtgtcagcggtttaccgatcgagtatgcaacggaagacggcaaatcaattatcaatatgggcggtaatttctatttggaagaacctgctaaagatggttcgattaagttaattccagtggtgaatgttaaaggtaaattctcaaccaaaacgcaaaatccagatggcagtattacgcttaagtcattagcagtaaaagtgaatttggcaaatgaaactccgatggtattaggtaatgtcgctgaaggggtagcagatacggacgctgttaatgtgaaacagttgaaatctgcgaaaactgaagtggaatctaccgatcacagtgtggtgattaaagagcgtcagggcgataatcagcaaatcgtgtatgatttggcggttgctaaaacgaaactcactgcctctaaggataaacgcaccattagtgcagcagataaaggcaaccattttgcgacaggagatgaagtcgcagtagcaattaataccgcaacagcagccgcaagaaccgaagttgaagcgggtaaaaatgtgaaagtgacttcaaaaacgggggcaaatggtcagaatatttacaatgtgagcgtgtttggagatttaagcgacattacttcaattagtaatggcgatacgaaagtatctttaggtaaagataagcaaggaaatccagttgtaaatatgaatggtgccagaattaccaacgttggagatggtagtgctgagggcgatattgtgaatgttcgtcagctcaacaaagtggtttcttctgtgaatacaggatttaatcaattatcaagagatattgttaatgcaagagcgggtattgcttctgctggggcgatggctaatttgccacaaatttctttaccaggtaaaagtgctatttctgtttctaatgcacaatatcgcgggcaatctgcctatgctataggttattccaaaatttctgataatggcaaatggcttattcgagcgtctgttagcagtaatactcagcgggatactatgattggaggaggggtaggttttgtgtggtaa

SEQ ID NO: 329 - DNA, 501 bp, synthetic construct
  source 1..501: mol_type = other DNA
aagcgaaagtagcagcaggtgaagctggtggtattactcagcatatcggtgcatatcacgttgaaaccgacgacggtaagatgattaccttcttagatacaccaggacacgcggcatttacctcaatgcgtgcgcgtggtgcgaaagcaacggatatcgttgttcttgtagtagcagctgacgatggcgtaatgccacaaaccattgaagcaatccaacacgcgaaagcagctggtgcgccgatcgtggttgcggtaaacaaaattgataaaccagaagcaaacctagagcgtgtagaacaagagttattacaacacgaagtgatttctgagaaattcggtggtgatgttcaatttgttcctgtttcagcgaaaaaaggaatggggattgacgacttacttgaagccattcttcttcaatcggaagtattagaattaagtgcggtaaaagagggtatggcaagcggtgtggttatcgaatcttacctcgataaaggtcgtg

SEQ ID NO: 330 - DNA, 1008 bp, synthetic construct
  source 1..1008: mol_type = other DNA
atgaaacaaatcgttttaaaaacaagcttattgatgaccctctcttcattattagttgcatgtagcggcggtggcggtagcgctggaaatcgtgctgaccgtgtagaggaaaaagcacaaccggttcaatcaaatagtgagccttcttccgctccaatcaaaaatcctactaataccgctacgaatgattctcttcatgacaaactttcaatgtcttctcatgacacatccaaagaaaatagtcaacaatcctcctttaaagcccctctagaacaagaaaaaaaccaacctgcacaagaaaatctcacttggacaggttatcatgtttcagaagtgggaaatgcgagtaataatgtagataaagataacgttacggtattcactttcgtaaaatataattctcaatacaatgatgatccagtttttgataaaacaaaaacacaaagtaaaacaatatcattagttgacggaaaaaatgagaataaagaggattattataactttacgttaaaagacgctttattttattatggaagttatggacaaccttcagcagattacaaaaaagtagaaaaaaattatatttatgcaattaaaccagatgcaataaataatgagaacctcaatgcactaactgcaacttattatcaagaagatggttttatatattccgtattaagtgatgtaaatcgagttggttcagaatatattcctcagtatggcaatgtgactcttactttccgaaatggcaagatttatggtgaaatctacagatataatagaggacgtgatgatttgtttcagctctcaggagaaggacaaaacttaactataacaccacacaaggacaatccccataaactatcccctacaggacccgacaacatggcaatggagctgaattttatcaacgcagaaaaaactgataaaaaatacgttgttggtgtaggaaaagctgaaaaatattatgggttattatttgctgaaaaaagtcaccaagcacaataa

SEQ ID NO: 331 - DNA, 1800 bp, synthetic construct
  source 1..1800: mol_type = other DNA
atgcattcaagtaaaccttatttttggggaagtgtgtttcttttgttcacgtttttaggtgcctatcttgcttcggcaatagatcgtgattttggtcgaattgaggtgagccatgtgagctttatgacagaagaaatgcaaccgatggtcgcgaagctatatcgtccagtttctgctacggcagacacaccgaagccaggacttttggcattgcatggctatcaaagtgataaagaagcaactagtacatttggtgcgttggagctggctaagcgcggttttgtggtgctagcgatcgatcattttggacatggctattctactaagctacccgcttcaaataaaaatatgagtggagcgaataatggttatcaatatttaaaaacgttgccgtttgtagataacacacggttaggtctctttggtcactctactggcgcgttaaatgcaattcgcgtggcaaaactcaatccagatcacaaagctgtcaatggattaagtagcaatggtggtgagatgacattgaataactacttattgacacaaggattgtacgaagaaattggtggctatcgtgaaagaaccttccctgtaaaaagtttgattcatcatcctaaccgcctgaaagcatttgggttaggcgaaaatgaaactttgcagtgggatcatacctatggtgactttaacaccggttctgcacgtcgtgcggcaatggtcgatggtacccatcttggggtgatgattgcttcccagagtaataaagaagccattctttggtttaatcaagcattgcaacatggcgaaaaaggtgcggattggattgatcctgaccaacagacctattggtataaagaacttacagggttatttgccttagcttgtgctttgcttgccacattatgttttgctagcggacttttaaaaacgacttattttggcgtgatgaatcaagcggtcacagaaaagacagcaatttcgagcaaacaatggtggggctttacgctgattaatattgtgctgacattactgctctatccgctatttacccagtggggcggtgcgaatgaaccgattgcagcgaaattgagttttatgccacttgaaatgggaaatggcattattttatggctcgttgtgagtgggcttgtcggtagtcttttatttggcttgtggcaaagaaaagcacagttttgttgggcggagtttggtgtgttgagccaatctgcttccttgacaacggcgcaactgattggacgttatttattactcagcttattgttatttgccggtttatatttccttgtcaatctgatttatcaatatttccatgttgagttacgtttcttatggccattattgaagccattaacgacagagcggtttaatttatttatcgtgtattggttacctattttggtctttttcttcgtgttcaacggtttgatcgtgtcagtccaaatgaaacaaaaagtagcgagttcgtttacggcaacattgctgatctggagtttcaaaaccgcactttttgctactggtggtttaatcattttatggttattccattttgttcccggttttatgcaaatcggtccgggatttgatgtggtaggactgccacaatttggtggacgttggatgatgatgttagccgtcattattccacagtttattgtcttcaccgtgatcaatcactggtgctatttaaaaacaggctatatttatttaggggtatttttcacctctctgttaatgacttgggtgttagtcggaggtcaagtcattgggcgattcttagcctaa

SEQ ID NO: 332 - DNA, 1098 bp, synthetic construct
  source 1..1098: mol_type = other DNA
atgaatattgcaacaaaattaatggctagcttagtcgctagtgtagtgcttaccgcatgtagtggcggcggctcatcgggttcatcgtctaaaccaaattcggaacttacacctaaggttgatatgtccgcaccaaaagcggagcagccaaaaaaagaggaagttccacaagcggataattcgaaagcggaagaaccaaaagagatggctccgcaagtagatagcccgaaagcggaagaaccaaaaaatatggctccacaaatgggtaatccaaaactaaatgacccacaagtaatggctccgaaaatggataatccgcaaaaagatgccccaaaaggagaagaactaagtaaggataaaagtaatgcggaaattcttaaggaattaggggttaaggatattaattcaggtatcattaataatgctgatgtagttctgaatttaaaaatagatgaaaaagatcacattacagtcgtattagataagggtaagattaatcgtaatcatctaaaagtaactaatacaatttctgctcaagacattaaaaccttaaaagattcttcaggcaaattgttgggttactatggatatatgcagttaaatcaagttcgacaagatgaaaattatagcgatgaaaaagttagtttgaatgaatattatttattatcaatgaacgatgccgataaaatacgtccgactaaatctatatcatataagggagacatgttttatagttacaaagatgtaggaaatcagaaattaaaggcttctgtagaagcttcttatgatgatgtaacaaaaaaagtatcaatgaaagtatttggtgagaataatgattactggaaattaggtgagtttggtagaactaatttattagaaaatcaagtgactggagcaaaagttggcgaagatggtaccattataaatggaactttatattctaaaatagataattttcctttaaaactaactcctgacgcaaacttctctgggggtattttcggtaaaaatggcgaagtattagccggaagtgctattagtgaaaaatggcaaggcgtaatcggtgctacggcaaccacaaaagaagataaaaaataa

SEQ ID NO: 333 - DNA, 5418 bp, synthetic construct
  source 1..5418: mol_type = other DNA
atgacaaaattaactatgcaagatgtgaccaatttatatttatataaaacgaaaactctacctaaagatagattggatgattcacttatttctgaaataggaaaaggagatgatgatattgatagaaaagaatttatggtggggccgggacgttttgtgaccgctgataactttagcgttgtaagagatttttttaatgctgggaaatcacgcattattgcgccgcaagtcccgcctattcgttcacagcaggaaaaaatcttggtcggtttaaaaccgggcaaatattccaaagcgcagatattggaaatgctgggttatacgaaaggcggagaagtggtaaatggcatgtttgccggtgaagtccagacattaggcttttatgacgatggcaaaggggatttactcgaacgcgcctatatctggaataccacaggatttaaaatgagcgacaatgccttttttgttatagaagaatcaggcaaacgctatattgaaaactttggtattgaacctcttggtaagcaagaagattttgattttgtcggcggcttttggtctaacttagtgaatcgtggtttggaaagtattatcgacccatccggtatcggtggaacggtaaaccttaactttaccggcgaggtggaaacctacacgttagacgaaacaaggtttaaagcggaagcggcgaagaaaagccattggagtttagtgaatgcggcgaaagtatacggcggtttagaccaaattattaaaaaactatgggacagtggctcaattaagcatttatatcaagataaagatacgggcaaattaaaaccgattatttacggcacggccggcaacgacagtaagattgaaggcactaaaatcacccgtaggattgcgggtaaagaagttacgcttgatattgccaatcagaaaattgaaaaaggcgtgttagagaaattggggctgtctgttagtggttcggatatcattaaattgttgtttggagcattgactccaactttaaatagaatgttgctatcacaacttatccagtctttttccgatagcttggctaaacttgataatcccttagccccttacactaaaaatggcgtggtttatgtcaccggcaaagggaatgatgtgcttaaaggaactgaacatgaggatttgtttctcggtggtgaggggaatgatacttattatgcgagagtaggcgatacaattgaagacgccgacggcaaaggtaaagtctattttgtgagagaaaaaggggtacctaaggcggatcctaagcgggtagagtttagcgagtacataacgaaagaagaaataaaagaggttgaaaaggggttattaacttacgcagttttagaaaattataattgggaagagaaaacggcgactttcgctcatgcgactatgcttaatgagctttttactgattatactaattatcgttatgaagttaaaggactaaaattgcccgccgttaaaaagttaaaaagtccgttggtggagtttacagctgatttattaactgttacgcctattgacgaaaacggaaaagcacttagcgaaaaaagtattacggttaaaaattttaaaaatggtgatttaggaataaggttgttggatcctaatagctattattatttccttgaaggccaagatacgggtttttatggtcctgctttttatattgaacgaaaaaacggtggcggcgctaaaaataactcgtcgggagcaggaaatagcaaagattggggcgggaacgggcatggaaatcaccgaaataatgcctccgacctgaataaaccggacggaaataatgggaataaccaaaataacggaagcaatcaagataatcatagcgatgtgaatgcgccaaataacccgggacgtaactatgatatttacgatcctttagctttagatttagatggagatgggcttgaaaccgtgtcgatgaacgggcgacaaggcgcgttattcgatcatgaaggaaaaggtattcgtaccgcaacgggctggctcgctgcggatgacggttttttagtgttagatcgtaaccaagacggcattattaatgatataagcgagttatttagtaataaaaatcaactttccgacggcagtatttctgcacacggttttgcgacattagccgatttggatacaaaccaagatcagcgtatcgaccaaaatgataagctgttttctaaactccaaatttggcgggatttaaatcaaaacggttttagtgaagcgaatgagctgtttagcttagaaagtttgaatattaaatctttacataccgcctatgaagagcgtaatgattttctagcgggcaataatatccttgctcagcttgggaagtatgaaaaaacggacggtacttttgcacaaatgggcgatttaaatttcagttttaacccgttttatagccgatttaccgaagcgttaaatttaaccgagcaacaacgtcgcacaattaatctaaccggcaccggtcgggttcgggatttgcgtgaagccgccgcactttctgaggagttggctgctttattacaacagtacactaaggcctccgattttcaggcacaacgagaattattgcctgccattttagataaatgggcggcaacggatttacagtatcaacattatgataaaacattacttaaaacggtagaaagtaccgatagtagtgcttctgtcgttagagtcacgccttctcaattaagtagtatacgcaatgcaaagcatgatcctaccgttatgcaaaactttgaacagagtaaggcaaaaattgcgactttaaattcgctctacgggttaaatatcgatcaactttattacacgacggataaagacattcgctatattactgataaagtgaataatatgtatcaaacaaccgtagaacttgcctaccgttctttacttttacaaacgcgtttgaagaaatatgtttatagcgttaatgcgaaacaattcgaagggaaatgggtaaccgattattctcgtactgaagccttatttaactctacttttaaacaatcgcctgaaaatgcattatatgatttaagcgaatacctttctttctttaacgatcctacggaatggaaagaagggctattactgttaagccgttatatagattatgctaaagcacaaggattttatgaaaactgggcggctacttctaacttaactattgcccgtttaagagaggctggagtaatttttgcagaatcgacggatttaaaaggcgatgaaaaaaataatattttgttaggtagccaaaaagataataacttatcgggtagtgcaggtgatgatctacttatcggcggagagggtaatgatacgttaaaaggcagctacggtgcagacacctatatctttagcaaaggacacggacaggatatcgtttatgaagataccaataatgataaccgcgcaagagatatcgacaccttaaaatttaccgatgtgaattatgcggaagtgaagtttcgacgagtagataatgacttaatgttattcggttatcatgatacggattcggtcacggtaaaatccttctacagccatgtagattatcaatttgacaaattggagtttgctgaccgcagtataactcgcgatgaactgattaaagcagggcttcatctatacggcaccgatggcaatgatgatataaaggatcatgcggattgggacagcattttggaaggcggcaaaggcaacgatattctaagaggtggctacggtgcggacacctatatctttagcaaaggacacggacaggatatcgtttatgaagataccaataatgataaccgcgcaagagatatcgacaccttaaaatttactgatgtgaattatgcggaagtgaaattccgacgagtagataatgacttaatgttattcggttatcatgatacggattcggtcacgataaaatccttctacaaccatgtagattatcaatttgacaaattggaatttgctgaccgcagtataactcgtgatgaactaggtaaacaaggtatggcattatttggcactgacggtgatgataatatcaacgactggggacgtaactcggtgattgatgccggtgcgggtaatgatacggttaatggcggtaatggcgatgacaccctcatcggcggcaaaggtaatgatattctaagaggtggctacggtgcggacacctatatctttagcaaaggacacggacaggatatcgtttatgaagataccaataatgataaccgcgcaagagatatcgacaccttaaaatttaccgatgtgaattatgcggaagtgaaattccgacgagtagataatgacttaatgttattcggttatcatgatacggattcggtcacggtaaaatccttctacagccatgtagattatcaatttgacaaattggagtttgctgaccgcagtataactcgcgatgaactgattaaagcagggcttcatctatacggcaccgatggcaatgatgatataaaggatcatgcggattgggacagcattttggaaggcggcaaaggcaacgatattctaagaggtggctacggtgcggacacctatatctttagcaaaggacacggacaggatatcgtttatgaagataccaataatgataaccgagcaagagatatcgacaccttaaaatttactgatgtgaattatgcggaagtgaaattccgacgagtagataatgacttaatgttattcggttatcatgatacggattcggtcacgataaaatccttctacaaccatgtagattatcaatttgacaaattggaatttgctgaccgcagtataactcgtgatgaactaggtaaacaaggtatggcattatttggcactgacggtgatgataatatcaacgactggggacgtaactcggtgattgatgccggtgcgggtaatgatacggttaatggcggtaatggcgatgacaccctcatcggcggcaaaggtaatgatattctaagaggtggctacggtgcggacacctatatctttagcaaaggacacggacaggatatcgtttatgaagataccaataatgataaccgcgcaagagatatcgacaccttaaaatttactgatattaatttatccgaactttggtttagccgagaaaataacgatttgattattaaatcattattaagtgaggataaagtcacggttcaaaattggtattcacaccaagatcataaaatagaaaatattcgtttatcgaatgagcaaacgttggtgagcactcaggtggagaagatggttgagtcgatggccggctttgctcagaagcacggaggagagatatctcttgtgtcgcttgaagaggtaaaacaatatatcaatagcttaacagctgctttataa

SEQ ID NO: 334 - DNA, 1347 bp, synthetic construct
  source 1..1347: mol_type = other DNA
atgtcaaatgccaaagcttacatccaagcttcttttgaagcagtcaaagcccgcaacccacatgaaacagaattcctccaagctgtagaagagctcttctctacacttgagcctgtttttgaagcacacccagaatacatcgaagaaaacatcttggctcgtatcgttgagcctgagcgtaccatcagcttccgtgttccatggacagataaagatggaaatgttcaagtcaaccgtggctaccgtgttcagttcaactcagctgtaggtccttataaaggcggtcttcgcttccacccaactgtaaaccaatccatcttgaagttcctcggttttgagcaaatcttcaaaaacgtcttgactggtcttccaatcggcggtggtaaaggtggttcagactttgatcctaaaggaaaaactgatgctgaaatcatgcgcttctgccaaagcttcatgactgaattgcaaaaacacatcggaccttcacttgacgtccctgctggtgacatcggtgtcggtggtcgtgagatcggttacatgtacggtcaatacaaacgcctccgccagtttgatgcaggtgtcttgactggtaaacctcttggcttcggtggttcattgatccgcccagaagcaactggttacggtttggtttacttcactgataacatgttggcagcaaacggtaaatccttcaaagaccaaactgtccttatctcaggttctggtaacgttgcccaatatgctgttcaaaaagcgactgaacttggtgcaaaagttatttctgtttcagactcaaatggttacatcattgacgaaactggtatcgacttcgacctcttggtggacatcaaagaaaaacgccgcgctcgtttgacagaatacgctgcagaaaaatcaactgctaagtacttcaaaggttctgtatggaactacgatggcaaggctgatattgcccttccatgtgcgactcaaaatgagatcaacggcaaacaagctgctgcccttgtaaaaaatggcgtgtactgtgtggctgaaggtgccaacatgccatctgaccttgatgccatcaaagtctacaaggaaaatggcgttctctacggactcgcaaaagctgccaacgctggtggtgtagctgtatctgcccttgaaatgagtcaaaacagccttcgcttgtcatggactcgtgaagaagtagacggccgtcttaaagacatcatggccaacatcttcaacacagccaaagaaactgctgaaaaatacgaccttggtacagactaccttgcaggtgctaacatcgcagcctttgaacaaattgcggatagcatgattgcccaaggtttggtataa

SEQ ID NO: 335 - DNA, 1662 bp, synthetic construct
  source 1..1662: mol_type = other DNA
atgttactagaagtgtccattaaaaacttcgccattattgagcaggtatcactgaattttgaaaacgggatgaccatcctatctggtgaaacaggtgcaggtaaatccattatcattgatgccatgaacctgatgctaggggcgcgtgcgacgacagatgttatcagacatggggccgccaaggctgagattgaagggctcttttcctttgaaaatagcagagctctcgagcaaattttgcttgaacaggggattgaagtagctgacgaactcattatccgccgtgaaatcctgcaaaatggccgttcggtcagccgtgtcaatggacaaatggtcaatttatctgtcttgaaacagattgggcaatatttagtagatattcatggtcaacatgaccaggaagaattgatgaaatcccagcaccatatccgtcttttggacagtttcggagaagatgaattttggagtcttaaagaccgttatcagacaacctttgatgcctatcgtagtcttcgcaaacgagttcttgaaaagcaaaaaaatgagcaagaacacaaggcgcggattgagatgctagaataccaaattgctgaaatcgaagcagcggatttgaagtctggggaagatattcaactcaatcaggaacgcgataaactgctcaaccacaaacaaattgcagatacactgaccaatgcctatgcactgttagacaatgaagatttttcaagcttgaacaacctacgctcagctatgagtgacttgcaaagtctggaagaatttgatccagactacaaacagctctcttctagcctgacagaagcttattatgtcgttgaagatataaccaagcgtctcagcgatgtggtcgataatctagattttgacggcaatcgtctcatgcaattggaaagtcgtctggatttgctcaataccattacgaagaaatatggtggaactgtcgatgatgttttggactattttagtaaaatcagcgaagaatacaatctattgacaggcaatgatttatctggagatgatttggaagttcagcttaagaacttagagaaagaattggttgaacgagcaggtcagctcagccaatcacgccatgaattggctgttgttttagaagatattatccgccaagaattgcaagacttgtatatggagaaagctcgtttccaagttcgatttactaaaggaaaatttaatcgagaaggaaatgaaacagtagaattttacatctctaccaacccaggtgaagacttcaagcctctggttaaagtggcttctggaggagaattgtctcgcttgatgttggccattaagtcagcctttgcccgtaaagaaggcaagacttccatcgtctttgacgaggtggatacaggggtgtctggacgtgtggcccaggccattgcccagaaaatttataaaatcggacaatatgggcaagtattggcgatttctcatctaccacaagtcattgctattgcagattatcagttctttattgagaaaatatccgacgaacattcgaccgtttctcgtgtccgtctcttgaccaaagaagagcgtattgaggaaattgccaagatgttagccggtgacaaaatcacagatgcagcccgtaatcaagcaaaagaattagtagaaaaaggttag

SEQ ID NO: 336 - DNA, 1012 bp, synthetic construct
  source 1..1012: mol_type = other DNA
atggtagttaaagttggtattaacggtttcggacgtatcggtcgtcttgctttccgtcgtatccaaaacgtagaaggtgttgaagttactcgtatcaacgaccttacagatccagtaatgcttgcacacttgttgaaatatgacacaactcaaggtcgtttcgacggtactgttgaagttaaagacggtggtttcgaagttaacggtaaattcgttaaagtttctgcagagcgcgagccaggaaacatcgactgggctactgatggcgtagatatcgttttggaagctacaggtttctttgcttctaaagaaaaagctgagcaacacatccacgctaacggtgctaaaaaagttgttatcactgctcctggtggtaacgatgttaagactgttgttttcaacactaaccacgacatccttgatggtactgaaacagttatctcaggtgcttcatgtactacaaactgtttggcaccaatggctaaagctcttcacgatgcatttggcgttcaaaaaggtttgatgactacaatccacggttacactggtgaccaaatggttcttgacggaccacaccgtggtggtgaccttcgtcgtgcacgtgctgctgctgcaaacatcgttcctaactcaactggtgctgctaaagctatcggcttggtaatcccagaattgaacggtaaacttgacggtgctgcacaacgtgttccagttccaacaggttctgtaactgaattggttgcaactcttgacaagaaagtaactgctgaagaagtaaacgctgctatgaaagctgctgctactgaatcatttggttacactgaagaccaactcgtatcttcagatatcgtaggtatctcattcggttcattgtttgatgcaactcaaactaaagttatcgaagttgatggcgagcaattggttaaagttgtttcatggtacgacaacgaaatgtcttacactgcacaacttgttcgtactcttgagtacttcgcaaaaattgctaaataat

SEQ ID NO: 337 - DNA, 495 bp, synthetic construct
  source 1..495: mol_type = other DNA
atgtcaaaaaataatatagtggagtgctttatagaaattgcaaagcactccaatcttaaatatgagtgtgttgatggcaaattgaaactagatagagtactatttggttcaatggtatatccgcacaactatggatatatttctgatactctagcagaggatggagatcctctagatgtagtagtgctatctaatttctctgtaactccaggaacttatttggattgcaaaattcttggttctctagaaatggtagattctggagaacaagattgaaaagttattgcaattatggatgcggatccaagactcaagcatataaattctctagatgatgttcctcaacattgaattgctgaattgagaaacttttttgaaagttacaagcaactagagaataagaaagtttcccttggaaactttatctctctagaatcaactctttctttaattgaagaatcaaaagctagatgaagaactcaagggggagaataa

SEQ ID NO: 338 - DNA, 1011 bp, synthetic construct
  source 1..1011: mol_type = other DNA
atgacaatccacaaagtagcaatcaatggattcggaagaatcggaagattactatttagaaatcttctttcttctcaaggagttcaagttgtagctgttaatgacgtagttgacattaaagttcttactcaccttttggtttatgacagtgctcaaggaaaactaaaagattgagaagtaagttgtgattcagaatacataagactaaagaatgtaaatactggagaagttagagaagttagagttttcaacttcaatactgaaaagatttatcactgaggtgaattagaaattgattgtgttgttgaatgttcaggaagattcttaactaaggaagcagttaagtgtcaccttgatgcaggagctcaaaaagttcttatttcagctcctgcaaaggatgacactaagacagttgtttacaacgtaaaccatactcaaattaccagctcagacaatgttatttcaggagcttcatgtacaactaatgcactagctcctatcgtaaaaattattcacagaaaatttggaattaattctggattcatgacaacagttcacgctttcacttctgaccaaagacttcaagactctcctcactctgacctaagaagagctagagcagctgctggatcaattattcctacaactacaggagcagctgctgcaattggaagagtaattccagaattgaatggaaaacttgatggaattgcacacagagtgcctgtattaactggttctctagttgacctatgtttaaaaataaataagtctgtttctgcagaagaaatcaatgaagcaattaaggatggagaaaatgaaacccttgcttatgtagaagatccaattgtatctgctgacattatcggagatacacatggttctgtttttgactcatctctaactaaagtattgccaactggagaagttaagctgtatgcatgatatgataatgagtcttcttatgtaaatcaacttgcaagaactttgaaatactacatttctctttaa

SEQ ID NO: 339 - DNA, 538 bp, synthetic construct
  source 1..538: mol_type = other DNA
ttcttcccagagtggatttccgttggttccgcctccttcgtccgtcgtaatatcaggccttctgttctgttcgctgtctgtctagggcacccttactgcaagagaagtatttgaggtcatatcgtcccatgaagtcgaccacctgtttcctctcttcactgtcacgtacgacatcgcattcaagggaagagatccagcagatctcgttcgtgtattcgagacaagagaggtccgcccccacaagacggctgaagaatgcaacattcttgtgctgcctcctctcatggcaaatgccagaagaagggtacgtgttgcatcataacaagagctgtatttcccgctggcaaatacaggtgaaatgtacctccagaaaagccacctagtatcgtgcggcaatgtgccacctcgcctcttgggagaaaaagaggaagagacgctgccgctgttttgcaaatgaaaaggattcattttcgcagtacaccaggagttggattttgtagagcgtctctcttcaagcagcgtattgtcgagaaaaaga

SEQ ID NO: 340 - DNA, 529 bp, synthetic construct
  source 1..529: mol_type = other DNA
ctgcagggaggaagacgaaagttgtttttttatttttttttctttttgtttttctgatttttgttttttttgactcgggcccagctgcgtctgtcgggatgagaccgcggagccgaagtgcgttttctttttttgacttttttttgttttttcacaggcaagctcgcctgtgcttggagccacagaagggacagaagtcgaaggggactacagacgcgatgccgctcctccagccgtcttggaggagagatatcaggactgtagatgaaggcgagggtgaggatgagggggtggcgtggttgggaagcgacgagagtcggagagggagaagatgtttccggcttggctgcttttcctggagggtggaaaaagagacaccggaatgcgatccagacgagacgacgctttcctcgtggtgatggcggagagaattgaagagtggagaagagggcgagggagacagagtcggaggcttggacgaagggaggaggaggggtaggagaggaatccagatgcactgtgtctgcag

SEQ ID NO: 341 - DNA, 1212 bp, synthetic construct
  source 1..1212: mol_type = other DNA
ttgctcaaaaaatttaaaaattttattctattttcatctatattttcaccaatagcatttgctatatcatgttctaatacaggagtagtcaagcaagaggatgtatcagttagtcaaggtcaatgagataaaagtataacatttggtgtttcagaagcttggttaaacaagaaaaaaggagataaagaagttaacaaagaagttattaatacatttttagaaaatttcaaaaaagaatttaataaactcaaaaatgcaaatgataaaaccaaaaacttcgatgacgttgattttaaagtaactccaattcaagactctactgtgttgttaaacaatttatctactgacaatcctgaattagattttggaattaatgcttcaggaaaattggtagaatttctaaaaaataatcctggtataataactcctgcattagaaacaacaactaattcttttgtatttgacaaagaaaaagataaattttatgttgatggtacagattcagatccacttgtaaaaattgctaaagaaattaataaaatttttgttgaaactccatatgcaagttgaactgatgaaaatcataagtgaaatggtaatgtttatcaaagtgtttacgatccaactgttcaagctaatttttatagaggaatgatttgaataaaaggtaatgatgaaactctagctaaaattaaaaaagcttgaaatgataaagattgaaatacatttagaaattttggaattttacacggtaaagataattcttcttctaaattcaagttagaagaaactatattaaaaaaccactttcaaaataaatttacaacactaaatgaagacagaagcgcacatccaaacgcatataaacaaaaatctgcagatacattgggaactttagatgatttccatattgctttttcagaagaaggttcttttgcttgaacacataacaaatcagcaacaaaaccttttgaaactaaagcaaatgaaaagatggaagcacttatagtaactaatccaattccgtatgatgttggagtgtttagaaaaagtgttaaccaattagaacaaaatttaattgttcaaacattcattaatttagctaaaaataaacaagatacatatggtccacttttagggtataatggttataaaaaaattgacaatttccaaaaagagattgtagaagtttatgaaaaagccattaaataa

SEQ ID NO: 342 - DNA, 1349 bp, synthetic construct
  source 1..1349: mol_type = other DNA
gatgaacgctcgctgtgtgcctaatacatgcatgttgaacgggatgtagcaatacattcagtagcgaatgggtgagtaacacgtacctaacctacctttaagactgggataactattggaaacaatagctaataccggatatagttatttatcgcatgatgagtaatagaaaggagcttcacagcttcacttaaaaatgggggtgcggaacattagttagttggtagggtaatggcctaccaagacgatgatgtttagccgggccgagaggctgtacggccacactgggactgagatacggcccagactcctacgggaggcagcagtaaggaattttccacaatgagcgaaagcttgatggagcgacacagcgtgcaggatgaagttcttcggaatgtaaactgctgttataagggaagaaaaaatagaataggaaatgattttatcttgacggtaccttattagaaagcgacggcaaactatgtgccagcagccgcggtaatacataggtcgcgagcgttatccggaattattgggcgtaaagcgtccgtaggttttttgctaagtctggagttaaatgctgaagctcaacttcagtccgctttggatactggcaaaatagaattataaagaggttagcggaattcctagtgaagcggtggaatgcgtagatattaggaagaacaccaataggcgaaggcagctaactggttatatattgacactaagggacgaaagcgtggggagcaaacaggattagataccctggtagtccacgccgtaaacgatgatcattagttggtggaataatttcactaacgcagctaacgcgttaaatgatccgcctgagtagtatgctcgcaagagtgaaacttaaaggaattgacgggaacccgcacaagcggtggagcatgtggtttaatttgaagatacgcgtagaaccttacccactcttgacatcttctgcaaagctatagagatatagtggaggttaacagaatgacagatggtgcatggttgtcgtcagctcgtgtcgtgagatgttaggttaagtcctgcaacgagcgcaacccttttctttagttactaatattaagttaaggactctagagatactgcctgggtaaccaggaggaaggtggggacgacgtcaaatcatcatgcctcttacgagtggggcaacacacgtgctacaatggtcggtacaaagagaagcaatatggtgacatggagcaaatctcaaaaaaccgatctcagttcggattgaagtctgcaactcgacttcatgaagtcggaatcgctagtaatcgtagatcagctacgctacggtgaatacgttctcgggtttt

SEQ ID NO: 343 - DNA, 948 bp, synthetic construct
  source 1..948: mol_type = other DNA
atgaaacctattaaaatagctctaattggtgctggaaatgtcggaaattctttcctttatgcagcaatgaatcaaggacttgcatccgagtatggaattattgatattaaccctgattttgccgatggtaatgcttttgattttgaagatgcctcagcttctttgccttttccgattagtgtctcccgttatgaatataaagatctaaaagatgctgattttattgtaattacagcgggaagaccacaaaaaccgggtgaaactcggcttgaattagtagctgataacatccgaattatccgggaaattgcactaaaagtcaaagaaagtggctttagtggaataagtattattgttgctaatcctgttgatataattacaagggcttaccgggatgcttctggattttccgatcaaaaagttatcggtagtggaactgttttagatacagcaaggcttcaatttgcaatcgcaaaaagagcaaaagtatcgcctaattcggttcaggcctacgtgatgggtgaacatggtgattcatcttttgttgcttattcaaatattaaaattgccggtgaatgtttctgtgcttattctaaactaaccggaattgatagctcaaattacgaaaaagaacttgaatatccagtttctcgccgggcttatgaaattattaatcgtaaaagggcaacattttatggaattggtgcagctattgccaaaatagtttctaatattatcaaagatacaaaaaatattatgattgccggagcaaatttacgaggagaatacggatttcacggagtaaatatcggagttccagttgttttaggggcaaacggaattgaaaaaattattgagattagttttaatgataaagaaaaagaaaaatttgccaaatcagttgcaatcattgataaaatttatcaggatgcaattaaaaatatttaa

SEQ ID NO: 344 - DNA, 2715 bp, synthetic construct
  source 1..2715: mol_type = other DNA
ttactttttaacatagtttctaatcaacccgttaattaattcacttctttttgttaaacttcctttctctgcaaatgatttatcattaaatattgccagcgtttttaatataattgtaggttcttttttagcgttttgttgtggttttttattttgttcgcctgggccgatttgataccaatcaagatgaaatcattctaaagtaaagaaatttggcaaggtgtctttagggttaatctcgaatttaagacgttttacacgttttgggtactttgttggaaaaaaataagaatttaccttaatcgaatccttgccagcaagaatttcatataaataggtaccattctcaacaatttctttggttttaggtacattattatttgaattttgaactttaattacaagcccttttccttttctatcggctaaaagattaatccaattaccatcggaaatattttttgcatcaaaaactaaataaattgctccggtatataaagaagttgatatttctttttctggattagacgattttgactgtaatttcaaaggtttctttagctctaaaccttgatcagtttgaattgcatcttctgatttaaatattaatttattatcacttaaatcttttaggataaattcttttttatcttttccttgattttgataattaattgttccatctaaaaacacatcgggattgaatttcttaattatatcataggcagatttattagaattaataatattattaagactaattttggactgagccaaaattagattttgattagttttatcaactaatttaaaatcaaaaaatagcttatttcctaaagttgagatctcatattcgctttttgtattttcaagattataaatattagcgaaattaaaagctgttcaaatgcttgtcccaaatttttcattttctaatacagaatttgagctgcttgtggaattaggaagtgaattttgactagtttgaggcgatgaagtggaaactggttgtgtttgctgactttgactttgacccttagcttcttctgcaaaagtgcttaccttgactgtttttgctgaattttctgttgttttagctagtgaaacctctggttttttcggttgtgtggggctatttttttctgttttttgagtatttgcctttgattttaagccctcaatcacggtttttagctcttgttttactttttcctggtcttggtcgactaaatattttcccgtatttaataaataaatgcttttaagaacttcttcacttggattttccaaattagtcactgtttttgctagttcaaaaagtcgatcaactcgctgttttatttcatttgaggataaagtttttgcatcttgatcttcaggcattgaaaattctttaactgcttttatcgttccaaacatttgggcaacagcggcttggtatttattaaaatcaacttttagatcaacaaaaactggaagatcctggatgataaattttttatcaatagcttttcgttttaaagaagcagtaatttcaaagcgaattttttgattttcaactagttttttcttatcaacaacagaaatctgacttggatctaaaagtttgggaataatacttaaatagacagttccgattttggtattaagaaaaaaatttgtctcacttaaatcaaaaagatctttaaattctttactttttgttaaaatttgggatgtatttacttttgaattcggagatccatatcaataatcagagaggctaattttatcttttactagtgccatttgcaaatcatctttggtttttaatttgccttgaagatttgtttcaagtcaggattttagaattttagaaatttcctgatttgaaatcgcgccgcgaacttcaagatttaaattcatagtttttttagttgaaatttgcactaaattaagtccaagaactaattttccatctttatttaacaaagttaataatttttttcggtccaaaactgggtctaattgataatctgggcctaaaaatacgggttctcctaaggaattataaagtgaaagactcgcttttgatgcaaccaaagctctttcaagtcgtgaaaatcaattacttcctctttcattttcaaaattttgggcaagcaaaatttggaattccatcaaagttagatttgctcttgaaagatcaacaaaagacttgcttgaatcaatttgactaaaatcaaaattagttctagcttgttctgtatttccaaagcctttaagtacaatttgttttgaataagttatttgatccttatcagattttgcataaataacaacatttttaattgaattttgatcaactactgcattttcaagatcaaaacttactaaataatcattgtttattagttcacttaaatcaaaagcagaaacaagattataacgataaagatcacttttggcagcagttaaaactgtttttgccgatcattttttaaaattatctttaattttaagatttaaaacaattgaatcaaatttttcctgactaattttagcattttttgctacttttagctgactcgggatctgatttagataagaataatatgatttattataagaattaattccgacaactgtaccaacagcggctgataaactaagactaattccggcaattgttgttattaatcaaaaaggttttttaagtaattttgctaacttcat

SEQ ID NO: 345 - DNA, 21 bp, synthetic construct
  source 1..21: mol_type = other DNA
gacatggctacgatccgactt

SEQ ID NO: 346 - DNA, 17 bp, synthetic construct
  source 1..17: mol_type = other DNA
cgcttggcctccgactt

(19) United States
(12) Patent Application Publication
(10) Pub. No.: US 2024/0167106 A1
(43) Pub. Date: May 23, 2024

(54) PRIMER GROUP FOR SIMULTANEOUS DETECTION OF 15 PORCINE PATHOGENS THROUGH HIGH-THROUGHPUT TARGETED AMPLICON SEQUENCING AND USE THEREOF

(71) Applicant: China Agricultural University, Beijing (CN)

(72) Inventors: Shaolin WANG, Beijing (CN); Yang ZUO, Beijing (CN); Zhangqi SHEN, Beijing (CN); Lei ZHOU, Beijing (CN); Jun HAN, Beijing (CN); Jianzhong SHEN, Beijing (CN)

(73) Assignee: China Agricultural University, Beijing (CN)

(21) Appl. No.: 18/509,322

(22) Filed: Nov. 15, 2023

(30) Foreign Application Priority Data
Nov. 18, 2022 (CN) 202211450766.0

(51) Int. Cl. C12Q 1/70; C12Q 1/6874; C12Q 1/6888

(52) U.S. Cl. CPC C12Q 1/701; C12Q 1/6874; C12Q 1/6888; C12Q 1/6844; C12Q 2600/16

(57) ABSTRACT

A primer group for simultaneous detection of 15 porcine pathogens through high-throughput targeted amplicon sequencing and use thereof are provided. The primer group for simultaneous detection of 15 porcine pathogens through high-throughput targeted amplicon sequencing includes: primers having nucleotide sequences set forth in SEQ ID NO: 1 to SEQ ID NO: 302. The nucleotide sequences of pathogenic target genes are set forth in SEQ ID NO: 303 to SEQ ID NO: 344. The primer group of the present invention is used for amplicon sequencing detection analysis method for 42 target genes of 15 porcine respiratory pathogens, has the technical advantages of rapidness, high efficiency, high targeting, strong specificity, and the like, and is suitable for rapid and accurate simultaneous diagnosis of multiple pathogens of diseased pigs in pig farms.

### CROSS REFERENCE TO THE RELATED APPLICATIONS

This application is based upon and claims priority to Chinese Patent Application No. 202211450766.0, filed on Nov. 18, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC153_Sequence Listing.xml, created on 10/26/2023, and is 379,175 bytes in size.

### TECHNICAL FIELD

The present invention relates to the technical field of high throughput of target gene detection, and in particular, to a primer group for simultaneous detection of 15 porcine pathogens through high-throughput targeted amplicon sequencing and use thereof.

### BACKGROUND

Amplicon sequencing is a high-targeting method, and the general idea is to target-capture a target region, then perform next generation sequencing (NGS) and analyze a sequencing result by designing primers near the conserved region of a target gene, performing PCR amplification and further performing sequencing analysis on an amplification product, thereby obtaining corresponding information.

Amplicon sequencing mainly comprises 16S rDNA sequencing, 18S rDNA sequencing, ITS sequencing, target region amplicon sequencing, and the like. At present, the amplicon sequencing is mainly applied to analysis of a metagenomics sample, and a sequence of a specific hypervariable region of 16S/18S/ITS determined by a second-generation high-throughput sequencing platform is used to reflect the differences between species of environmental samples in the classification of bacteria, fungi, and archaea, so that the microbial composition in the environments such as ocean, soil, and intestinal excrement is studied. In addition, few studies apply amplicon sequencing to pathogen detection, however, there is no related design for detecting porcine respiratory pathogens at present.

The respiratory diseases of pigs are mainly caused by single or mixed infection of viruses, bacteria and other pathogens, and the diagnosis methods of the pathogens generally comprise nucleic acid detection, antigen detection, and the like. Generally, most of detection means are single aiming at a certain pathogen, and the simultaneous rapid and accurate diagnosis of a plurality of pathogens cannot be implemented. In addition, at present, the most extensive fluorescent quantitative detection means for detecting the epidemic diseases in the pig farms can only detect 3 to 5 pathogens at most, however, the fluorescent quantitative detection means is only a simple diagnosis of pathogenic genes, and cannot further detect pathogenic gene mutations on the basis of disease diagnosis so as to discover a new mutant pathogen.

### SUMMARY

An objective of the present invention is to provide a primer group for simultaneous detection of 15 porcine pathogens through high-throughput targeted amplicon sequencing, which is used for amplicon sequencing detection analysis method for 42 target genes of 15 porcine respiratory pathogens, has the technical advantages of rapidness, high efficiency, high targeting, strong specificity, and the like, and is suitable for rapid and accurate simultaneous diagnosis of multiple pathogens of diseased pigs in pig farms.

In order to achieve the above objective, the present invention provides the following technical solution.

The present invention provides a primer group for simultaneous detection of 15 porcine pathogens through high-throughput targeted amplicon sequencing, which comprises primers having nucleotide sequences set forth in SEQ ID NO: 1 to SEQ ID NO: 302, the primer group is used to amplify pathogenic target genes, the pathogenic target genes are as follows:

| Pathogen name | Target gene | Sequence No. |
|---|---|---|
| African swine fever virus | ASFV-p72 | SEQ ID NO: 303 |
| | ASFV-p54 | SEQ ID NO: 304 |
| | ASFV-EP402R | SEQ ID NO: 305 |
| | ASFV-MGF505-1R | SEQ ID NO: 306 |
| | ASFV-MGF505-2R | SEQ ID NO: 307 |
| | ASFV-MGF360-13L | SEQ ID NO: 308 |
| | ASFV-MGF360-14L | SEQ ID NO: 309 |
| | ASFV-K205R | SEQ ID NO: 310 |
| Porcine reproductive and | PRRSV-NA-orf6/M | SEQ ID NO: 311 |
| respiratory syndrome virus | PRRSV-NA-orf7/N | SEQ ID NO: 312 |
| | PRRSV-EU-orf6/M | SEQ ID NO: 313 |
| | PRRSV-EU-orf7/N | SEQ ID NO: 314 |
| Pseudorabies virus | PRV-gE | SEQ ID NO: 315 |
| | PRV-gB | SEQ ID NO: 316 |
| Porcine circovirus | PCV2-cap | SEQ ID NO: 317 |
| | PCV2-rep | SEQ ID NO: 318 |
| | PCV3-cap | SEQ ID NO: 319 |
| | PCV3-rep | SEQ ID NO: 320 |
| Classical swine fever virus | CSFV-5′UTR | SEQ ID NO: 321 |
| | CSFV-E2 | SEQ ID NO: 322 |
| Foot and mouth diseases | FMDV-3D- | SEQ ID NO: 323 |
| virus | 1C2B(polyprotein) | |
| | FMDV-5′UTR | SEQ ID NO: 324 |
| Swine influenza A virus | swIAV-M | SEQ ID NO: 325 |
| | swIAV-H1 | SEQ ID NO: 326 |
| | swIAV-H3 | SEQ ID NO: 327 |
| Haemophilus parasuis | HPS-vtaA | SEQ ID NO: 328 |
| | HPS-infB | SEQ ID NO: 329 |
| Pasteurella multocida | Pm-plpE | SEQ ID NO: 330 |
| | Pm-kmt1 | SEQ ID NO: 331 |
| Actinobacillus | APP-omlA | SEQ ID NO: 332 |
| pleuropneumoniae | APP-apxIVA | SEQ ID NO: 333 |
| Streptococcus suis | S.suis-gdh | SEQ ID NO: 334 |
| | S.suis-recN | SEQ ID NO: 335 |
| | S.suis-gapdh | SEQ ID NO: 336 |
| Eperythrozoon suis | E.suis-ppa | SEQ ID NO: 337 |
| | E.suis-g1 | SEQ ID NO: 338 |
| Toxoplasma gondii | T.gondii-B1 | SEQ ID NO: 339 |
| | T.gondii-RE | SEQ ID NO: 340 |
| Mycoplasma hyorhinis | Mhr-p37 | SEQ ID NO: 341 |
| | Mhr-16SrRNA | SEQ ID NO: 342 |
| Mycoplasma | Mhp-p36 | SEQ ID NO: 343 |
| hyopneumoniae | Mhp-p102 | SEQ ID NO: 344; |

nucleotide sequences of the pathogenic target genes are set forth in SEQ ID NO: 303 to SEQ ID NO: 344;

SEQ ID NO: 303:
atggcatcaggaggagctttttgtcttattgctaacgatgggaaggccgacaagattatattggcccaagacttgctgaatagcaggatct
ctaacattaaaaatgtgaacaaaagttatgggaaacccgatcccgaacccactttgagtcaaatcgaagaaacacatttggtgcattttaa
tgcgcattttaagccttatgttccagtagggtttgaatacaataaagtacgcccgcatacgggtacccccaccttgggaaacaagcttacc
tttggtattccccagtacggagactttttccatgatatggtgggccatcatatattgggtgcatgtcattcatcctggcaggatgctccgattc
agggcacgtcccagatgggggcccatgggcagcttcaaacgtttcctcgcaacggatatgactgggacaaccaaacacccttagagg
gcgccgtttacacgcttgtagatccttttggaagacccattgtacccggcacaaagaatgcgtaccgaaacttggtttactactgcgaata
ccccggagaacgactttatgaaaacgtaagattcgatgtaaatggaaattccctagacgaatatagttcggatgtcacaacgcttgtgcg
caaattttgcatcccaggggataaaatgactggatataagcacttggttggccaggaggtatcggtggagggaaccagtggccctctcc
tatgcaacattcatgatttgcacaagccgcaccaaagcaaacctattcttaccgatgaaaatgatacgcagcgaacgtgtagccatacca
acccgaaatttctttcacagcattttcccgagaactctcacaatatccaaacagcaggtaaacaagatattactcctatcacggacgcaac
gtatctggacataagacgtaatgttcattacagctgtaatggacctcaaacccctaaatactatcagccccctcttgcgctctggattaagtt
gcgcttttggtttaatgagaacgtgaaccttgctattccctcagtatccattcccttcggcgagcgctttatcaccataaagcttgcatcgca
aaaggatttggtgaatgaatttcctggactttttgtacgccagtcacgttttatagctggacgccccagtagacgcaatatacgctttaaacc
atggtttatcccaggagtcattaatgaaatctcgctcacgaataatgaactttacatcaataacctgtttgtaacccctgaaatacacaacctt
tttgtaaaacgcgttcgcttttcgctgatacgtgtccataaaacgcaggtgacccacaccaacaataaccaccacgatgaaaaactaatgt
ctgctcttaaatggcccattgaatatatgtttataggattaaaacctacctggaacatctccgatcaaaatcctcatcaacaccgagattggc
acaagttcggacatgttgttaacgccattatgcagcccactcaccacgcagagataagctttcaggatagagatacagctcttccagacg
catgttcatctatatctgatattagccccgttacgtatccgatcacattacctattattaaaaacatttccgtaactgctcatggtatcaatcttat
cgataaatttccatcaaagttctgcagctcttacatacccttccactacggaggcaatgcgattaaaacccccgatgatccgggtgcgatg
atgattacctttgctttgaagccacgggaggaataccaacccagtggtcatattaacgtatccagagcaagagaattttatattagttggga
cacggattacgtggggtctatcactacggctgatcttgtggtatcggcatctgctattaactttcttcttcttcagaacggttcagctgtgctg
cgttacagtacctaa;
SEQ ID NO: 304:
atggattctgaattttttcaaccggtttatccgcggcattatggtgagtgtttgtcaccagtcactacaccaagcttcttctccacacatatgta
tactattctcattgctatcgtggtcttagtcatcattatcatcgttctaatctatctattctcttcaagaaagaaaaaagctgctgctattgagga
ggaagatatacagtttataaatccttatcaagatcagcagtgggtagaagtcactccacaaccaggtacctctaaaccagctggagcga
ctacagcaagtgtaggcaagccagtcacgggcagaccggcaacaaacagaccagcaacaaacaaaccagttacggacaacccagt
tacggacagactagtcatggcaactggcgggccggcggccgcacctgcggccgcgagtgctcctgctcatccggctgagccttaca
cgacagtcactactcagaacactgcttcacaaacaatgtcggctattgaaaatttacgacaaagaaacacctatacgcataaagacctag
aaaactccttgtaa;
SEQ ID NO: 305:
atgataatacttatttttttaatattttctaacatagttttaagtattgattattgggttagttttaataaaacaataattttagatagtaatatta
ctaatgataataatgatataaatggagtatcatggaatttttttaataattcttttaatacactagctacatgtggaaaagcaggtaacttttgtg
aatgttctaattatagtacatcaatatataatataacaaataattgtagcttaactatttttcctcataatgatgtatttgatacaacatatcaagt
agtatggaatcaaataattaattatacaataaaattattaacacctgctactcccccaaatatcacatataattgtactaattttttaataacatgt
aaaaaaaataatggaacaaacactaatatatatttaaatataaatgatacttttgttaaatatactaatgaaagtatacttgaatataactggaata
atagtaacattaacaattttacagctacatgtataattaataatacaattagtacatctaatgaaacaacacttataaattgtacttatttaacatt
gtcatctaactatttttatactttttttaaattatattatattccattaagcatcataattgggataacaataagtattcttcttatatccatcata
acttttttatctttacgaaaaagaaaaaaacatgttgaagaaatagaaagtccaccacctgaatctaatgaagaagaacaatgtcagcatgatgaca
ccacttccatacatgaaccatctcccagagaaccattacttcctaagccttacagtcgttatcagtataatacacctatttactacatgcgtcc
ctcaacacaaccactcaacccatttcccttacctaaaccgtgtcctccacccaaaccatgtccgccacccaaaccatgtcctccacctaa
accatgtccttcagctgaatcctattctccacccaaaccactacctagtatcccgctactacccaatatcccgccattatctacccaaaatat
ttcgcttattcacgtagatagaattatttaa;
SEQ ID NO: 306:
atgttctctctccagaacttatgtcgaaaaacattacctaaccgtaaacttcctgaattttttgacgaatatatattacaactgctgggattata
ctgggaaaaccatggaactattcaacgagcaggaaacaactgtgtgcttatacagcaacataccctcattcccgtaaatgaagccctga
gaacagcagcatctgaagaaaattatgagatcgtgagccttttattagcgtgggaggggaacctttactatgctattataggggctctaga
gggcaaccgccacgacttaattcgtaaatatgatgaccaaatcaaggaccatcatgaaattctgccattcattgacgatccagtcatatttc
acaaatgccatatcatgcggcaatgcttttttgattgtattttatatcaagctgtaaaatatagtaagtttcgcgttcttctttactttaaacatag
attagaggatgatttgcccttcactcatttacttattgaaaaggcatgtaaagatcataattatgaagttattaaatggatatatgaaaacctac
atatctacaatatgatagatacctttgaatgtgctattgcccataaggatctacatctatattgtttggggtatagatttatatataacagaatcg
tacccgataagtatcatcatttagatattcgcatgctttcaagcctacaactcctacataaggtggcagccaaaggatacttagattttatcct
agaaaccttaaagtatgatcataataaagataatataaatattattctaacacaagctgcaacctataaccatagaaaaattttaatctatttca
ttcctcaatcaacccacgcacagatagaacaatgtttactagtggcgataaaagcaaaatcttccaggaaaaccttgaacttactactgtct
cacctaaacctttccatcaacctcatcaaaaaaataagccattatgttgccacttacaattcaacaaatataataggcattctgagtatgcgg
cggaaaaagaagatatatttagatatcatattgacaaaatttgtaaaaaaagctatttttaataagtttgtcgttcgatgtatggatacattttct
ataaacccggaaagaatccttaaaatagccgcgcgaataaataggatgatgttagtgaaaaaaatatctgaacatgtttggaaaaatcat
gcggttagacttaaataccttaaacatgcggtacacacgatgaagcataaagatgggaaaaatagactcatgaactttatctatgatcgct
gttattaccatatgcaaggggaagaaatctttagcctcgcaagattttatgcaatccatcatgcaccaaagttgtttgacgttttttatgattgt
tgtatcctagatacgatacgattcaaaagccttcttttagattgttcacatatcataggtaaaaacgctcatgatgctaccaatatcaacatcg
tgaacaagtatatcggcaacctgtttgttatgggagttcttagcaaaaaagaaatcttacaggactatccatccatttattctaaacaatacat
gccttag;
SEQ ID NO: 307:
atgttttcccttcaagacctttgccgaaagcatctttttattcttcccgatgtttttggcgagcatgtactacaacgattaggactgtattggag
atgtcacggctcccttcaacgcataggagacgaccacatactcatacgacgggatctcatcctttccaccaacgaggccttaagaatgg
cgggagaggaaggaaacaatgaagtagtaaagctcttgttactgtggaagggaaatcttcattacgccgtcataggagccttgcagggt
gatcaatatgacctgatccataagtatgaaaaccaaatcggcgactttcattttatcttaccattgattcaagacgcgaatacgtttgaaaaa
tgccacgctttagaacgtttttgtggtgtttcatgtctgctaaaacatgctacaaaatacaacatgctccctattctccaaaaataccaagaa
gagctgtctatgagagcgtatcttcacgaaaccctatttgaactagcatgcctatggcagaggtatgatgtccttaaatggatagagcaaa
ccatacatgtttacgacctaaagattatgtttaatattgccatctccaagagggatctgactatgtactccttaggatatattttcctttttgatag
agggaacaccgaagctacgttgctaacgcaacatctcaagaagacagcggccaaagggctcctccactttgtgctagaaacgttaaaa
tacggcggcaacatagataccgtcctgacccaagccgtaaagtacaatcatagaaaacttttagattattttctgcgtcaactacctcgtaa
acatattgaaaaacttttgttgctggccgtgcaggaaaaggcttctaaaaaaacattgaacttactgttgtcacatttaaactactccgtgaa
acgcatcaaaaaactaccgcgctatgtgatagagtacgagtccaccttggtgataaagattttattaaaaaaaagagtgaacctgatagat
gccatgttggaaaagatggtaagatatttttctgcgacgaaagtgaggacgatcatggatgagctttcgattagtccggaaagagtcatta
agatggctatacagaaaatgagaacggatatcgtaatccatacttcttatgtttgggaggatgatctagaacgtcttactcgtcttaaaaata
tggtatacaccataaagtacgaacatgggaaaaaaatgttaattaaagtcatgcacggcatatacaaaaacttattatacggcgaaaggg
aaaaagtcatgttttatttagccaagctctatgttgctcaaaacgcggccacccaattcagagacatttgtaaggactgttacaaactggat
gtggcacggtttaaaccgcggtttaagcaactaatattagactgtttagaaattattactaaaaaatcttgctatagtatcctggaaatcttag
aaaaacatattatttccctgtttactatgaaagttatgactgaagaagaaaaaaacctatgtttagaaatattatataaagtaattcattataaa
acaatacaatgttaa;
SEQ ID NO: 308:
atgtcgttgccgctctctctgcagaccctcgtcaaaaagacgatagccagccagtgtttgtcaatagatgaacactgcattttgaaatattg
tggcctatggtggcatgatgctcctctcaagctttgtatggatcgtggccgaatacaaataaaatcaggatttttaggagaagatatagac
cttcgtgtggcattaataatagctgttaaggaaaacaactatagtctgataaagctctttacagagtggggcgcaaatatcaactatggttt
gctttctatcaatacgaagcacatccgagagttgtgtagacagctaggcgccaaagaaactctagaggacaacgatattttccgtattttta
ccaggataatgcacaataaaaccagcggcagtattattttgtgccatgaaatttttatgaataatcctattttagaaaacaaatttgttatacaa
ttaaggggcttaatttataaaagactatgggggctcatagaaataaaagaaacggacgagttaaatggtttactagtgaagtattggtacg
ccaaagcagtacaatacgattgtaaggacgccatttgttttctagatgagaaatatacggatcttaatgaatggcgattaaaatgtctcctgt
attataacaaaatatatgagcttcatgagatgtaccacaaggaaaacatccaaatagacgtccatgacatgatatgtctggcttctaccaa
ggataacaatccattaacaatatattactgttacgcgctggggggcaacatcaaccaagctatgcttacttcagtacaatattataacatcg
gtaatatatttttctgtatagatttgggtggtaatgcctttgaagagggtcgtgccatagcggaacaaaaaggttataattttctgagccatag
tttggctttggatatttacagctcagatgcttccttgccactaaacttaaaggaccccgaagaaataagcagtttattaaaagattataaatca
aaaaacttatccatcatttgggaatattctcataatatactatag;
SEQ ID NO: 309:
atgttgtctttacaaacgttggccaaaaaagttgtggcatgcaattatctttcaagtgactatgattatacgttgcagcgttttggtttgtggtg
ggatttaggtcctattcacctatgtaacaattgtaagcaagttttttcgtataaacatttacagtgtttttctgaggatgatctttgtctcgaagc
ggcgctagtaaaggccgtgaagagcgataatcttgaacttatacgtttatttgtggattggggcgcaaatcctgaatatgggcttatacgt
gttcctgccgtgtatctaaagcggctgtgtgcggaactgggaggcttaacgcctgtatccgaaccccgtcttctggaaattttaaaagaa
gtggccaggctaaaatcctgtgcaggagttctgctgggttatgacatgttttgtcataatccactcttggaaaccgtaactagaaccacttt
agacacagttacgtacacctgttcaaacattccgttgacgggggatacggcgcaccacctattaacaaagttttggtttgccctggcatta
cgacataattttacaaaggctattcactatttctataaaaggcataaaaatcacctctattgggggtagcttgtagcctttattttaataacatt
tttgacatacacgagttgtgtcgtgaaaaagagatttgcatcagccctaatctgatgatgaaatttgcttgcttgcgggaaaaaaattacgc
ggccatttattactgtcataggttgggggctagtctcgattatggcatgaatctttctatctataacaataatactttaaacatgtttttctgtatt
gatttggggggctgccgattttga;
SEQ ID NO: 310:
atggttgagccacgcgaacagttttttcaagacctgctttcagcagtggatcaacaaatggacactgtaaaaaatgacataaaagacatc
atgaaagaaaaaacatcttttatggtgtcattcgaaaactttatagaacgttacgataccatggaaaaaaatattcaagaccttcagaataa
gtacgaagaaatggcggccaaccttatgaccgtcatgacggatacaaaaattcagcttggagccattatcgcccaacttgagattctgat
gataaatggcactccacttccggcaaaaaaaacaacgattaaggaggctatgcccctaccttcatcaaacacgaacaatgatcaaacg
agtcctcccgcctcaggcaaaacaagtgaaacacctaaaaaaaatcccacgaatgcaatgttcttcacgcgtagcgaatgggcatcctc
gaaaacttttcgagaaaagtttttaacaccagaaattcaggccatattggatgagcagtttgcaaacaagaccgggatcgaaagattgca
tgccgagggtctttacatgtggagaacccaattctctgacgaacagaagaaaatggtcaaagagatgatgaagaagtaa;
SEQ ID NO: 311:
atggggtcgtctctagacgacttctgcaatgatagcacagctccacagaaggtgcttttggcgttttccattacctacacgccagtgatgat
atatgctctaaaggtaagtcgcggccgactgctagggcttctgcaccttttgatctttctgaattgtgcttttaccttcgggtacatgacattc
gtgcactttgagagcacaaatagggtcgcgctcactatgggagcagtagttgcacttctttggggagtgtactcagccatagaaacctg
gaaattcatcacctccagatgtcgtttgtgcttgctaggccgcaagtacattctggcccctgcccaccacgtcgaaagtgccgcgggctt
tcatccgattgcggcaaatgataaccacgcatttgtcgtccggcgtcccggctccactacggtcaacggcgcattggtgcccgggttga
aaagcctcgtgttgggtggcagaaaagctgttaagcagggagtggtaaaccttgttaaatatgccaaataa;
SEQ ID NO: 312:
atgccaaataacaacggcagacagcaaaataaaaagaagggggatggccagccagtcaatcagctgtgccagatgctgggcaaaat
tatcgcccagcagaaccagtccagaggtaagggaccggggaagaagaataagaatagaaacccggagaagccccatttccctctag
cgactgaagatgacgtcagacatcacttcaaccccagtgaacgacaattgtgcctgtcgtcaatccggactgcctttaaccaaggcgct
gggacttgcaccctgtcagactcagggagaataagttacactgtggagtttagtttgcctactcatcacactgtgcgcttgattcgcgcca
cagcgtcaccctcagcatga;
SEQ ID NO: 313:
atgggaagcctagatgatttctgcttcgatcacaccgccgcacaaaagcttgtgctagcctttagcatcacatatacacctataatgatata
cgccctcaaggtgtcacgcggccgactcctggggctgttgcacatcctgatatttctgaactgtgccttcacgttcggatacatgacatat
gtgcattttgaatccaccaaccgtgtcgcactcactatgggggctgttgttgcccttctgtggggtatttatagcttcacagagtcatggaa
gttcattacttccagatgcagattgtgttgcctaggccggcgatacattctggcccctgcccaccacgtagaaagtgctgcaggtctccat
tcaatcccagcgtctggtaaccgagcatacgctgtgagaaagcccggactaacatcagtgaacggcaccctagtaccagggcttcgg
agcctcgtgttgggcggcaaacgagctgttaaacgaggagtggttaacctcgtcaagtatggccggtaa;
SEQ ID NO: 314:
atggccggtaagaaccagagccagaagaaaaggaaaaacacagctccaatggggaatggccagccagtcaatcaactgtgtcagtt
gctgggtgcaatgataaagtcccagcgccagcaatctaggggaggacaggccaaaaagaaaaagcctgagaagccgcattttcccc
tggccgctgaagatgacatccggcaccatctcacccagaccgaacgatccctttgcttgcaatcgatccagacggcttttaatcaaggc
gcaggaactgcgtcgctttcatccagcgggaaggttggttttcaggttgagtttatgctgccggttgctcatacagtgcgcctgattcgcg
tgacttctacatccgccagtcagggtgcaagttaa;
SEQ ID NO: 315:
atgcggccctttctgctgcgcgccgcgcagctcctggcgctgctggccctggcgctctccaccgaggccccgagcctctccgccgag
acgaccccgggccccgtcaccgaggtcccgagtccctcggccgaggtctgggacgacctctccaccgaggccggcgacgatgacc
tcaacggcgacctcgacggcgacgaccgccgcgcgggcttcggctcggccctcgcctccctgagggaggcgcccccggcccatct
ggtgaacgtgtccgagggcgccaacttcaccctcgacgcgcgcggcgacggcgccgtgctggccgggatctggacgttcctgccc
gtccgcggctgcgacgccgtgtcggtgaccacggtgtgcttcgagaccgcgtgccacccggacctggtgctgggccgcgcctgcgt
ccccgaggccccggagatgggcatcggcgactacctgccgcccgaggtgccgcggctccggcgcgagccgcccatcgtcacccc
ggagcggtggtcgccgcacctgagcgtcctgcgggccacgcccaacgacacgggcctctacacgctgcacgacgcctcggggcc
gcgggccgtgttctttgtggcggtgggcgaccggccgcccgcgccggcggacccggtgggccccgcgcgccacgagccccgctt
ccacgcgctcggcttccactcgcagctcttctcgcccggggacacgttcgacctgatgccgcgcgtggtctcggacatgggcgactcg
cgcgagaactttaccgccacgctggactggtactacgcgcgcgcgcccccgcggtgcctgctgtactacgtgtacgagccctgcatct
accacccgcgcgcgcccgagtgcctgcgcccggtggacccggcgtgcagcttcacctcgccggcgcgcgcgcggctggtggcgc
gccgcgcgtacgcctcgtgcagcccgctgctcggggaccggtggctgaccgcctgccccttcgacgccttcggcgaggaggtgcac
acgaacgccaccgcggacgagtcggggctgtacgtgctcgtgatgacccacaacggccacgtcgccacctgggactacacgctcgt
cgccaccgcggccgagtacgtcacggtcatcaaggagctgacggccccggcccgggccccgggcaccccgtggggccccggcg
gcggcgacgacccgatctacgtggacggcgtcacgacgccggcgccgcccgcgcgcccgtggaacccgtacggccggacgacg
cccgggcggctgtttgtgctggcgctgggctccttcgtgatgacgtgcgtcgtcggggggccgtctggctctgcgtgctgtgctcccgg
cgccgggcggcctcgcggccgttccgggtgccgacgcgggcgcggacgcacatgctctctccggtgtacaccagcctgcccacgc
acgaggactactacgacggcgacgacgacgacgaggaggcgggcgtcatccgccggcggcccgcctcccccggcggagacagc
ggctacgaggggtcgtacgcgagcctggaccccgaggacgagttcagcagcgacgaggacgacgggctgtacgtgcgccccgag
gaggcgccccgctccggcttcgacgtctggttccgcgatccggagaaaccggaagtga;
SEQ ID NO: 316:
atgcccgctggtggcggtctttggcgcgggccccgcgggcatcggcccgggcaccacggcggtgctggcctcggacgtctttggcc
tgctccacaccacgctgcagctgcgcggggcgccgtcgcgctagcgctgctgctgctggcgctcgccgcgaccccgacgtgcggc
gcggcggccgtgacgcgggccgcctcggcctcgcccgcgcccgggacgggcgccaccccagacggcttctccgcggaggagtc
cctcgaggagatcgacggggccgtcacccccggcccctcgaacgcccccgacggcgagtacggcgacctggtcgcgcgcacggc
cgtgcgcgcggccgcgaccgagcgggaccgcttctacgtctgcccgccgccgtccggctccacggtggtgcgcctggagcccgag
caggcctgccccgagtactcgcaggggcgcaacttcacggaggggatcgccgtgctcttcaaggagaacatcgccccgcacaagtt
caaggcccacatctactacaagaacgtcatcgtcacgaccgtgtggtccgggagcacgtacgcggccatcacgaaccgcttcacgga
ccgcgtgcccgtccccgtgcaggagatcacggacgtgatcgaccgccgcggcaagtgcgtctccaaggccgagtacgtgcgcaac
aaccacaaggtgaccgccttcgactgcgacgagaaccccgtcgaggtgggcctgcgcccctcgcgcctgaacgcgctcggcaccc
gcggctggcacaccaccaacgacacctacaccaagatcggcgccgcgggcttctaccacacgggcacctccgtcaactgcatcgtc
gaggaggtggaggcgcgctccgtgtacccctacgactccttcgccctgtccacgggggacatcgtgtacatgtcccccttctacggcc
tgcgcgagggggcccacggggagcacatcggctacgcgcccgggcgcttccagcaggtggagcactactaccccatcgacctgga
ctcgcgcctccgcgcctccgagagcgtgacgcgcaactttctgcgcacgccgcacttcacggtggcctgggactgggcccccaaga
cgcggcgcgtgtgcagcctggccaagtggcgcgaggccgaggagatgatccgcgacgagacgcgcgacgggtccttccgcttcac
gtcgcgggccctgggcgcctccttcgtcagcgacgtcacgcagctcgacctgcagcgcgtgcacctgggcgactgcgtcctccgcg
aggcctcggaggccatcgacgccatctaccggcggcgctacaacaacacgcacgtgctggccggcgacaagcccgaggtgtacct
cgcccgcgggggcttcgtggtggccttccgcccgctgatctcgaacgagctggcgcagctgtacgcgcgcgagctcgagcgcctcg
gcctcgccggcgtcgtgggccccgcgtcccccgcggccgcccgtcgggcccggcgctcccccggcccggggggacgcccgag
ccgccggccgtcgacggcacggggcacctgcgcatcaccacgggctcggccgagtttgcgcgcctgcagttcacctacgaccacat
ccaggcgcacgtgaacgacatgctgagccgcatcgcggccgcctggtgcgagctgcagaacaaggaccgcaccctgtggggcga
gatgtcgcgcctgaaccccagcgccgtggccacggccgcgctgggccagcgcgtctcggcgcgcatgctcggcgacgtgatggcc
atctcgcggtgcgtggaggtgcgcggcggcgtgtacgttcagaactccatgcgcgtgcccggcgagcgcggcacgtgctacagccg
cccgctggtgaccttcgagcacaacggcacgggcgtgatcgagggccagctcggcgacgacaacgagctcctcatctcgcgcgac
ctcatcgagccctgcaccggcaaccaccggcgctactttaagctgggcggcgggtacgtgtactacgaggactactgctacgtgcgc
atggtggaggtgcccgagacgatcagcacgcgggtgaccctgaacctgacgctgctcaaggaccgcgagttcctgccccccgaggt
gtacacgcgcgaggagctcgccgacacgggcctcctggactacagcgagatccagcgccgcaaccagctgcacgcgctcaagttct
acgacattgaccgcgtggtcaaggtggaccacaacgtggtgctgctgcgcggcatcgccaacttcttccagggcctcggcgacgtgg
gcgccgccgtcggcaaggtggtcctgggcgccacgggggccgtgatctcggccgtcggcggcatggtgtccttcccgtccaacccc
ttcggggcgctcgccatcgggctgctggtgctggccggcctggtcgcggccttcctggcctaccggcacatctcgcgcctgcgccgc
aaccccatgaaggccctgtaccccgtcacgacgaaggcgctcaaggaggacggcgtcgaagaggacggcgtggacgaggccaa
gctggaccaggcccgggacatgatccggtacatgtccatcgtgtcggccctcgagcagcaggagcacaaggcgcgcaagaagaac
agcgggcccgcgctgctggccagccgcgtcggggcgatggccacgcgccgccggcactaccagcgcctcgagaacgaggaccc
cgacgccctgtag;
SEQ ID NO: 317:
atgacgtatccaaggaggcgttaccgaagaagaagacaccgcccccgcagccatcttggccagatcctccgccgccgcccctggct
cgtccacccccgccaccgttaccgctggagaaggaaaaatggcatcttcaacacccgcctctcccgcaccttcggatatactgtcaag
cgaaccacagtcagaacgccctcctgggcggtggacatgatgagattcaatattaatgactttcttcccccaggagggggctcaaaccc
ccgctctgtgccctttgaatactacagaataagaaaggttaaggttgaattctggccctgctccccgatcacccagggtgacaggggag
tgggctccagtgctgttattttagatgataactttgtaacaaaggccacagccctcacctatgacccctatgtaaactactcctcccgccat
accataacccagcccttctcctaccactcccggtactttacccccaaacctgtcctagattccactattgattacttccaaccaaacaacaa
aagaaaccagctgtggctgagactacaaactgctggaaatgtagaccacgtaggcctcggcactgcgttcgaaaacagtatatacgac
caggaatacaatatccgtgtaaccatgtatgtacaattcagagaatttaattttaaagaccccccacttaacccttaa;
SEQ ID NO: 318:
atgcccagcaagaagagtggaagaagcggaccccaaccacataaaaggtgggtgttcacgctgaataatccttccgaagacgagcg
caagaaaatacgggagctcccaatctccctatttgattattttattgttggcgaggaaggcaatgaggagggccgaacaccccacctac
aggggttcgctaattttgtgaagaagcaaacttttaataaagtgaagtggtattttggtgcccgctgccacatcgagaaagcgaaaggaa
cagatcagcagaataaagaatattgcagtaaagaaggcaacttactgatagaatgtggagctcctagatctcaaggacaacggagcga
cctctctaccgctgtgagtaccttgttggagagcgggagtctggtgaccgttgcagagcagcaccctgtaacgtttgtcagaaatttccg
cgggctggctgaacttttgaaagtgagcgggaaaatgcagaagcgtgattggaagacgaatgtacacgtcattatggggccacctgg
gtgtggaaaaagcaaatgggctgctaattttgcagacccggaaaccacatactggaaaccacctagaaacaagtggtgggatggttac
catggtgaagaagtggttgttattgatgacttttatggctggctgccgtgggatgatctactgagactctgtgatcgatatcctttgactgttg
agactaaaggtggaactgtaccttttttggcccgcagtattctgattaccagcaatcagaccccgttggaatggtactcctcaactgctgtc
ccagctgtagaagctctctatcggaggattacttccttggtattttggaagaatgctacagaacaatccacggaggaagggggccagttc
gtcaccctttcccccccatgccctgaatttccatatgaaataaattactga;
SEQ ID NO: 319:
atgagacacagagctatattcagaagaagaccccgcccaaggagacgacgacgccacagaaggcgctatgtcagaagaaaactatt
cattaggaggcccacagctggcacatactacacaaagaaatactccaccatgaacgtcatttccgttggaacccctcagaataacaagc
cctggcacgccaaccacttcattacccgcctaaacgaatgggaaactgcaattagctttgaatattataagatactaaagatgaaagttac
actcagccctgtaatttctccggctcagcaaacaaaaactatgttcgggcacacagccatagatctagacggcgcctggaccacaaac
acttggctccaagacgacccttatgcggaaagttccactcgtaaagttatgacttctaaaaaaaaacacagccgttacttcacccccaaa
ccaattctggcgggaactaccagcgctcacccaggacaaagcctcttctttttctccagacccaccccatggctcaacacatatgacccc
accgttcaatggggagcactgctttggagcatttatgtcccggaaaaaactggaatgacagacttctacggcaccaaagaagtttggatt
cgttacaagtccgttctctaa;
SEQ ID NO: 320:
gtccggagggaaagcccgaaacacaggtggtgttttacgataaacaactggaccccgaccgagtgggaatctattgtggagtgtgga
ggcagtatagcgagataccttattatcggcaaagaggttggaaaaagcggtaccccacacttgcaagggtacgtgaatttcaagaaca
aaaggcgactcagctcggtgaagcgcttacccggatttggtcgggcccatctggagccggcgagggggagccacaaagaggccag
cgagtattgcaagaaagagggggattacctcgagattggcgaagattcctcttcgggtaccagatcggatcttcaagcagcagctcgg
attctgacggagacgtcgggaaatctgactgaagttgcggagaagatgcctgcagtatttatacgctatgggcggggtttgcgtgattttt
gcggggtgatggggttgggtaaaccgcgtgattttaaaactgaagtttatgtttttattggtcctccaggatgcgggaaaacgcgggaag
cttgtgcggatgcggctgcgcgggaattgcagttgtatttcaagccacgggggccttggtgggatggttataatggggagggtgctgtt
attctggatgatttttatgggtgggttccatttgatgaattgctgagaattggggacaggtaccctctgagggttcctgttaagggtgggttt
gttaattttgtggctaaggtattatatattactagtaatgttgtaccggaggagtggtattcatcggagaatattcgtggaaagttggaggcct
tgtttaggaggttcactaaggttgtttgttggggggaggggggggtaaagaaagacatggagacagtgtatccaataaactattga;
SEQ ID NO: 321:
atggagttgaatcattttgaacttttatacaaaacaaacaaacaaaaaccaatgggagtggaggaaccggtatacgatgccacggggag
gccattgtttggagacccgagtgaggtacacccacaatcaacactgaagctaccacatgatagggggagaggtaacatcaaaacaac
actgaagaacctacctaggaaaggcgactgcaggagtggcaaccatctaggcccggttagtgggatatatgtaaagcccggccctgt
cttttatcaggactacatgggcccggtctaccatagagcccctctagagttttttaacgaagcgcagttttgcgaggtgaccaaaaggata
ggtagggtgacaggtagtgacggaaagctttaccatatatatgtgtgcatcgatggttgcatactgctgaagctagccaagagggacga
gccaagaaccctgaagtggattagaaatttcaccgactgtccattgtgggttaccagttgctctgatgatggcgcaagtggaagtaaaga
gaagaagccagataggatcaacaaaggcaaattaaaaatagccccaaaagagcatgagaaggacagcagaactaagccacctgac
gctacgattgtagtggaaggagtaaaataccaggttaaaaagaagggtaaagttaaaggaaagagtacccaagacggcctgtaccac
aacaagaataaaccaccagaatctaggaagaaattagaaaaagccctattggcatgggcggtaatagcaattatgttgtaccaaccagt
tgaagccgaaaatataactcaatggaacctgagtgacaacggcactaatggtatccagcatgctatgtaccttagaggggttagcagga
gcttgcatgggatctggccggaaaaaatatgcaaaggagtccccacctacctggccacagacacggaactgaaagaaatacaggga
atgatggatgccagcgaggggacaaactatacgtgctgtaagttacagagacatgaatggaacaaacatggatggtgtaactggtaca
atatagacccctggatacagttgatgaatagaacccaagcaaacttggcagaaggccctccggccaaggagtgcgctgtgacttgcag
gtacgataaagatgctgacatcaacgtggtcacccaggccagaaacaggccaacaaccctgaccggttgcaagaaaggaaaaaattt
ttcttttgcgggtacagttatagagggcccatgtaatttcaatgtttccgtggaggatatcttgtatggggatcatgagtgcggcagtttgctt
caggacacggctctgtacctagtggatggaatgaccaacactatagagaatgccagacagggagcagcgagggtaacatcttggctc
gggaggcaactcagcactgccgggaagaggttggagggtagaagcaaaacctggtttggtgcctatgccctatcgccttactgtaatg
taacaagcaaaatagggtacatatggtacactaacaactgcaccccggcttgcctccccaaaaatacaaagataataggccccggaaa
atttgacactaacgcggaagacggaaagattctccatgagatggggggtcacctatcagaatttctgctgctctctctggttgttctgtctg
acttcgcccctgaaacagccagcgcgttatacctcattttgcactacatgattcctcaatcccatgaagaacctgaaggctgcgacacaa
accagctgaatctaacagtggaactcaggactgaagacgtaataccgtcatcagtctggaatgttggcaaatatgtgtgtgttagaccag
actggtggccatatgaaaccaaggtggctttgttatttgaagaggcaggacaggtcgtaaaattagccttacgagcgctgagggatttaa
ccagggtctggaatagcgcatcaaccacggcattcctcatctgcttgataaaagtattaagaggacagatcgtgcaaggtgtgatatgg
ctgctactagtaactggggcacaaggccggctagcctgcaaggaagattacaggtacgcaatatcatcgaccaatgagatagggctac
tcggggccgaaggtctcaccaccacctggaaagaatacaaccacgatttgcaactgaatgacgggaccgttaaggccatttgcgtggc
aggttcctttaaagtcatagcacttaatgtggtcagtaggaggtatttggcatcattgcataaggaggcttcactcacttccgtgacatttga
gctcctgttcgacgggaccaacccatcaactgaggaaatgggagatgacttcgggttcgggctgtgcccgttcgatacgagtcctgttg
tcaagggaaagtacaatacaaccttgttgaacggtagtgctttctatcttgtctgcccaatagggtggacgggtgtcatagagtgcacag
cagtgagcccaacaactctgagaacagaagtggtaaagaccttcaggagagacaagccctttccgcacagaatggattgtgcgacca
ccacagtggaaaatggagatttattctactgtaagttggggggcaactggacatgtgtgaaaggtgaaccagtggtctacacgggggg
gctagtaaaacaatgcagatggtgtggcttcgacttcaatgagcccgacggactcccgcactaccccataggtaagtgcatcttggtaa
atgagacaggttacagaatagtagattcaacggactgtaacagagatggcgttgtaatcagcacagatgggagtcatgagtgcttgatc
ggtaacacaactgtcaaggtgcatgcatcagatgaaagactgggccctatgccatgcagacccaaagagattgtctctagtgcaggac
ctgtaaggaaaacttcctgtacattcaactacgcaaaaactttgaagaacaagtactatgagcccagggacagctacttccagcaatatat
gcttaagggcgagtatcagtactggtttgacctggacgtgactgaccgccactcagattacttcgcagaatttgtcgtcttggtagtggta
gcactgttaggaggaagatatgtcctgtggctaatagtgacctacatagttctaacagaacaactcgccgctggtttaccattgggccag
ggtgaggtagtgttgatagggaacttaattacccacacagacattgaggtcgtagtatatttcttactactctatttggtcatgagggatgag
cctataaagaaatggatactgctgctgttccatgctatgactaacaatccagtcaagaccataacagtggcattgcttatggttagtggggt
tgccaagggtggaaagatagacggcggttggcagcggctgccagagaccagctttgacatccaactcgcgctgacagttatagtagt
cgctgtgatgttactggcaaagagagatccaactactgtccccttggttataacagtggcaaccctgagaacggctaagatgactaatgg
acttagcacggatatagccatagctacagtgtcaacagcgttgctaacctggacctacattagtgactattatagatacaagacttggcta
cagtaccttattagcacagtgacaggtatcttcttaataagggtactgaagggaataggtgagttggatttacacactccaaccttgccatc
ttacagacccctcttcttcattctcgtgtacctcatttccactgcagtggtaacaagatggaatctggacatagccggattgctgttgcagtg
tgtcccaacccttttgatggtttttacgatgtgggcagacattctcaccctgatcctcatactgcccacttacgagctaacaaaactatattac
ctcaaggaagtgaagactggggcagaaaagggctggttatggaagaccaacttcaagagggtaaacgacatatacgaagttgaccaa
tctggtgaaggggtttaccttttcccgtcaaaaaaaagacaagttcaataacaggtaccatgttgccattgatcaaagccatactcatca
gctgcatcagtaataagtggcagttcatatatctattgtacttgatatttgaagtgtcttactacctccacaagaagatcatagatgaaatagc
aggagggaccaacttcatctcaagacttgtagccgctttgatcgaagccaattgggcctttgacaacgaagaagttaggggtttaaaga
agttcttcctgttgtctagtagggttaaagaactgatcatcaaacacaaagtgaggaatgaagtaatggtccactggtttggtgacgaaga
ggtttatgggatgccaaagttggttggcttagtcaaggcagcaacattgagtaaaaataaacattgtattttgtgcaccgtctgtgaagaca
gagagtggagaggagaaacctgcccaaaatgcgggcgttttgggccaccaatgacctgtggtatgaccctagccgactttgaagaaa
aacattataagaggatcttttttagagaggatcaatcagaagggccggttagagaggagtacgcagggtatctgcaatatagagccaga
gggcaattattcctgaggaatctcccggtgctagcaacaaaagtcaagatgctcctggtcggaaatcttgggacggaggtgggagactt
ggaacaccttggctgggtccttagggggcctgccgtttgcaagaaggttaccgaacatgagaaatgcaccacatccataatggacaaa
ttgactgcttttttcggtgttatgccaaggggcaccacacctagagcccctgtgagattccccacctctctcttaaagataagaagggggtt
ggaaactggctgggcgtacacacaccaaggtggcattagttcagtggaccatgtcacttgtgggaaagacttgctggtatgtgacacta
tgggccggacaagggtcgtttgccaatcaaataataagatgacagatgagtctgagtatggagttaaaactgactccggatgcccgga
aggagctaggtgttatgtgttcaacccagaggcagttaacatatcagggactaaaggagccatggtccacttacaaaaaactggagga
gaattcacctgtgtgacagcatcaggaactccggccttctttgatctcaagaacctcaaaggctggtcagggctaccgatatttgaggca
tcaagtggaagggtagtcggcagggtcaaggtcgggaagaatgaggactctaaaccaaccaagcttatgagtggaatacaaacagtc
tccaaaagtaccacagacttgacagaaatggtaaagaaaataacgaccatgaacaggggagaattcagacaaataacccttgctacag
gtgccggaaaaaccacggaactccctaggtcagtcatagaagagatagggaggcataagagagtcttggtcttgatccctctgagggc
ggcagcagagtcagtataccaatatatgagacaaaaacatccaagcatcgcatttaacctgaggataggggagatgaaggaagggga
catggccacagggataacctatgcttcatacggttacttctgtcagatgccacaacctaagttgcgagccgcgatggttgagtactccttc
atatttcttgacgagtaccactgtgccaccccagaacaattggccatcatgggaaagatccacagattttcagagaacctgcgggtagta
gccatgaccgcaacaccagcaggcacagtaacaaccacagggcagaaacaccctatagaagaattcatagccccagaagtgatgaa
aggggaagacttaggctcagagtacttggacattgctggactaaagataccagtagaggagatgaagagcaacatgctggtttttgtgc
ccactaggaacatggcggtggagacagcaaagaaattgaaagctaagggttacaactcaggctactattatagtggagaggatccatc
taacctgagggtggtaacgtcgcagtccccgtacgtggtggtggcaaccaacgcgatagaatcaggtgttactctcccggacttggatg
tggttgtcgatacagggcttaagtgtgaaaagagaatacggctgtcacctaagatgcccttcatagtgacgggcctgaagagaatggct
gtcacgattggggaacaagcccagagaagggggagagttgggagagtaaagcctgggagatactacaggagtcaagaaactcccg
ttggttctaaagattaccattatgatctactgcaagcacagaggtacggtattgaagatgggataaacatcaccaaatcctttagagagat
gaactatgattggagcctttatgaggaggacagtctgatgattacacaattggaaatcctcaataatttgttgatatcagaagaactaccga
tggcagtaaaaaatataatggccaggactgaccacccagaaccaattcagctggcgtacaacagctacgaaacacaagtgccagtgc
tattcccaaaaataaagaatggagaggtgactgatagttacgataactataccttcctcaacgcaagaaaattgggggatgatgtacccc
cttacgtgtatgccacagaggatgaggacttagcggtagagctactgggcttagactggccagaccctggaaaccaaggaaccgtag
aggctggcagagcactaaaacaagtagttggtctatcaacagctgagaatgccctgttagtagccttattcggctatgtaggatatcagg
cactttcaaagaggcatataccagtagtcacagatatatattcaattgaagatcacaggttggaagacaccacacacctacagtacgccc
caaatgctatcaagacggaggggaaggagacagaattgaaggagctagcccagggggatgtgcagagatgtgtggaagctatgac
caattatgcaagagagggtatccaattcatgaagtctcaggcactgaaggtgaaagaaacccccacttacaaagagacaatgaacact
gtgactgactatgtaaagaaattcatggaggcgctggcagacagtaaagaagacatcttaagatatgggttgtgggggacgcacacag
ccttatataagagcatcagtgccaggcttgggagtgagactgcgttcgctaccctggtcgtgaagtggctggcatttgggggggaatca
atagcagaccatgtcaaacaagcggccacagacttggtcgtctactatatcatcaacagacctcagttcccaggagacacagagacac
aacaggaaggaaggaaatttgtggccagcctactggtctcagctctagctacttacacatacaaaagctggaattacaataatctgtcca
agatagttgaaccggctttggccactctgccctatgccgccacagctctcaaactattcgcccccactcgattggagagcgttgtcatatt
gagtaccgcaatctacaagacctacctatcaatcaggcgcggaaaaagcgatggtttgctaggcacaggggttagtgcggctatggag
atcatgtcacaaaatccagtatccgtgggcatagcagtcatgctaggggtaggggccgtggcagcccacaatgcaatcgaggccagt
gagcagaagagaacactactcatgaaagtttttgtaaagaacttcttggaccaagcagccactgatgaattagtcaaggagagtcctga
gaaaataataatggctttgtttgaagcagtgcagacagtcggtaaccctcttagactagtataccacctttatggagttttctataaggggtg
ggaggcaaaagagttggcccaaaggacagccggtaggaaccttttcactttgataatgttcgaggctgtggaactactgggagtagata
gtgaaggaaagatccgccagctatcaagtaattacatactagagctcctgtataagttccgtgacagtatcaagtctagcgtgagggaga
tggcaatcagctgggcccctgcccctttcagctgtgattggacaccgacggatgacagaatagggctcccccaagacaatttcctccaa
gtggagacgaaatgcccctgtggttacaagatgaaggcagttaagaattgtgctggagagctgagactcttggaggaggaaggctcat
ttctctgcagaaataaattcgggagaggttcacggaactacagggtgacaaaatactatgatgacaatctatcagaaataaagccagtga
taagaatggaagggcatgtggaactctactacaagggagccaccatcaaactggacttcaacaacagtaaaacaatactggcaaccg
ataaatgggagattgatcactccactctggtcagggtgctcaagaggcacacaggggctggatatcatggggcatacctgggcgaga
aaccgaactacaaacatctgatagagagggactgtgcaaccatcaccaaagataaggtttgttttctcaaaatgaagagagggtgtgca
tttacttatgacttatcccttcacaaccttacccgactgattgaattggtacacaagaataacttggaagacaaagagattcctgctgttacg
gttacaacctggctggcttacacgtttgtaaatgaagatatagggaccataaaaccagccttcggggagaaagtaacaccggagatgc
aggaggaaataaccttgcagcctgctgtagtggtggatacaactgacgtgaccgtgactgtggtaggggaagcccctactatgactac
aggggagactccgacagcgttcaccagctcaggttcagacccgaaaggccaacaagttttaaaactgggggtaggtgaaggccaata
ccccgggactaatccacagagggcaagcctgcacgaagccatacaaggtgcagatgagagaccctcggtgctgatattagggtctga
taaagccacctctaatagagtgaaaactgcaaagaatgtaaaggtatacagaggcagggacccactagaagtgagagatatgatgag
gaggggaaagatcctggtcatagccctgtctagggttgataatgctctattgaaatttgttgactacaaaggcacctttctaactagagag
accctagaggcattaagtttgggtaggcctaaaaagaaaaacataaccaaggcagaagcgcagtggttgctgtgcctcgaagaccaa
atggaagagctacccgattggttcgcagccggggaacccatttttctagaggctaacattaaacatgacaggtaccatctggtggggga
tatagctaatatcaaggaaaaagccaaacagttgggagctacagactccacaaagatatctaaggaggttggtgcaaaagtgtattctat
gaaactgagtaattgggtgatgcaagaagaaaataaacagggcaacctgacccccttgtttgaagagctcctgcaacagtgtccaccc
ggaggccagaacaaaactgcacatatggtctctgcttaccaactagctcaagggaactggatgccaaccagctgccatgtttttatggg
gaccatatctgccaggaggaccaagacccatccatatgaagcatacgtcaagttaagggagttggtagaggaacacaagatgaaaac
attgtgtcctggatcaagcctgggtaagcacaacgaatggataattggtaaaatcaaataccagggaaacctgaggaccaaacacatgt
tgaaccccggcaaggtggcagagcaactgtgcagagagggacacagacacaatgtgtataacaagacaataggctcagtaatgaca
gctactggtatcaggttggagaagttgcccgtggttagggcccagacagacacaaccaacttccaccaagcaataagggataagatag
acaaggaagagaacctacaaaccccgggtttacataagaaactaatggaagttttcaatgcattgaaacgacccgagttagagtcctcc
tacgatgccgtggaatgggaggaactggagagaggaataaacaggaagggtgctgctggtttctttgaacgcaaaaatataggggaa
atattggattcagagaaaaacaaagtcgaagagattattgacaatctgaaaaaaggcagaaacatcaaatactatgaaaccgcgatccc
aaagaatgagaagagggacgtcaatgatgactggactgctggtgacttcgtggaagagaagaaacccagagtcatacaataccctga
agcaaaaacaaggctggccatcaccaaggtgatgtataagtgggtgaagcagaagccagtagttatacccgggtatgaagggaaga
cacctctattccaaatttttgacaaagtaaagaaggaatgggatcaattccaaaatccagtggcagtgagttttgacactaaggcgtggga
cacccaggtaaccacaaaagatttggagttgataaaggacatacaaaagtactatttcaagaagaaatggcataaatttattgacaccct
gaccatgcacatgtcagaagtacccgtaatcagtgctgatggggaagtatacataaggaaagggcaaagaggcagtggacaacctga
cacaagcgcaggcaatagcatgctaaatgtgttaacaatgatttacgccttctgcgaggccacgggagtaccctacaagagcttcgaca
gggggcaaaaattcatgtgtgtggggatgatggtttcctgatcacagaaagagctctcggtgagaaattcgcgagtaagggagtccag
atcctatatgaagctgggaagccccagaagatcactgaaggggacaagatgaaagtggcctaccaatttgatgatattgagttttgctcc
catacaccaatacaagtaaggtggtcagataacacttctagttacatgccggggagaaatacaaccacaatcctggctaaaatggccac
aaggttagattccagtggtgagaggggtaccatagcatatgagaaagcagtagcattcagcttcctgctgatgtactcctggaacccact
aatcagaaggatctgcttactggtgctatcaactgaactgcaagtgaaaccagggaagtcaaccacttactactatgaaggggacccga
tatctgcctacaaggaagtcatcggccacaatctttttgatcttaagagaacaagcttcgagaagctggccaagttaaatctcagcatgtc
agtactcggagcctggactagacacaccagtaaaagactactacaagactgtgtcaatgtgggtgttaaagagggcaactggctagtta
atgcagatagactagtaagtagcaagactggaaataggtacatacccggagagggccacaccctgcaagggagacattatgaagaa
ctggtgttggcaagaaaacagatcaacaactttcaagggaccgacaggtacaatctaggcccaatagtcaatatggtgttaaggaggct
gagagtcatgatgatgaccctgatagggagaggggtatga;
SEQ ID NO: 322:
cggctagcctgcaaggaagattacaggtacgcaatatcatcgaccaatgagatagggctactcggggccgaaggtctcaccaccacc
tggaaagaatacaaccacgatttgcaactgaatgacgggaccgttaaggccatttgcgtggcaggttcctttaaagtcatagcacttaat
gtggtcagtaggaggtatttggcatcattgcataaggaggcttcactcacttccgtgacatttgagctcctgttcgacgggaccaacccat
caactgaggaaatgggagatgacttcgggttcgggctgtgcccgttcgatacgagtcctgttgtcaagggaaagtacaatacaaccttg
ttgaacggtagtgctttctatcttgtctgcccaatagggtggacgggtgtcatagagtgcacagcagtgagcccaacaactctgagaaca
gaagtggtaaagaccttcaggagagacaagccctttccgcacagaatggattgtgcgaccaccacagtggaaaatggagatttattcta
ctgtaagttggggggcaactggacatgtgtgaaaggtgaaccagtggtctacacgggggggctagtaaaacaatgcagatggtgtgg
cttcgacttcaatgagcccgacggactcccgcactaccccataggtaagtgcatcttggtaaatgagacaggttacagaatagtagattc
aacggactgtaacagagatggcgttgtaatcagcacagatgggagtcatgagtgcttgatcggtaacacaactgtcaaggtgcatgcat
cagatgaaagactgggccctatgccatgcagacccaaagagattgtctctagtgcaggacctgtaaggaaaacttcctgtacattcaact
acgcaaaaactttgaagaacaagtactatgagcccagggacagctacttccagcaatatatgcttaagggcgagtatcagtactggtttg
acctggacgtgactgaccgccactcagattacttcgcagaatttgtcgtcttggtagtggtagcactgttaggaggaagatatgtcctgtg
gctaatagtgacctacatagttctaacagaacaactcgccgctggt;
SEQ ID NO: 323:
atgaacacagctggttgttttatcgctttgttgtacaccatcagagagataaaaacacgactgttttcaaggacacaggaagaaatggaat
tcacactttacaacggtgagaagaagatcttctactccaggcccaacaaccacgacaactgttggctgaacgccatccttcagctgttca
ggtacgtcgatgaacctttcttcgactgggtatatgaatcacctgaaaacctcacccttgaggcgatcagacaactggagaacattactg
gtcttgagctgcacgagggtggtccgcccgccctcgtcatttggaacatcaaacacttgctccacaccgggatcggcaccgcctcgcg
acccagcgaggtgtgcatggtggacggtacggacatgtgcctggctgacttccacgctggcatcttcctgaaaggacaggaacacgc
cgtgtttgcctgcgtcacctccaacgggtggtacgcgatcgacgacgaagaattctacccctggacgccagatccgtccgacgtgctg
gtctttgtcccgtacgatcaagaaccacttaatggggaatggaaagcaagggttcagagacggctcaagggagccggacaatccagt
ccggctactgggtcacagaaccaatcaggcaacaccgggagtatcatcaacaattactacatgcagcagtaccagaactccatggaca
cccaacttggtgacaatgctatcagcggaggctccaacgagggatccacagacacaacctccacccacacaaccaacactcagaac
aatgactggttttcaaagttggccagctctgccttcagcggtctcttcggcgccctcctcgccgataagaaaaccgaggagaccactctt
ctcgaggaccgcatcctcaccacccgaaacggacacaccacctcgacaacccagtcgagtgttggcataacgcacgggtacgcaac
agctgaggactttgtgaacgggccaaacacctctggtcttgagaccagagttgtccaggcggaacggttctttaaaacccacctgttcg
actgggtcaccagtgatccgttcggacggtactacttgttggagctcccgactgaccacaaaggtgtctacggcagcctgaccgactca
tacgcctacatgagaaacggttgggatgttgaagtcaccgctgtggggaatcagttcaacggaggctgcctactggtggccatggtgc
ctgaactttgttccatcgagcggagagagttgttccagcttacgctcttcccccatcagttcatcaacccccggacgaacatgacagccc
acatcaaggtgccctttgttggcgtcaaccgttacgatcagtacaaggtacacaagccgtggacccttgtggttatggtcgtagccccac
tgactgtcaacaccgaaggcgctccgcaaatcaaggtgtatgccaacatcgcgcccaccaacgtgcacgtcgcgggtgagttcccttc
caaagaggggattttccctgtggcatgtagcgacggttatggcggcttggtgacaactgacccaaagacggctgaccccgtttacggc
aaagtgttcaaccccccccgcaacatgttgccggggcggttcaccaacctcctggacgtggctgaggcttgccccacgtttctgcactt
cgatggtgacgtaccgtatgtgaccactaagacggattcggacagggtgctcgcacaatttgacttgtctttggcagcaaaacacatgtc
aaacaccttccttgcaggtcttgcccagtactacacgcagtacagcggcaccgtcaacctgcacttcatgttcgcaggtcccactgacg
cgaaagcgcgttacatgattgcgtatgcccctccgggcatggagccgcccaaaacacctgaggctgctgctcactgcattcacgcaga
gtgggacacgggtctgaactcaatgtttaccttttccatcccctacctctcggcggctgattacgcgtacaccgcgtctgacgctgctgag
accacaaatgttcagggatgggtctgcttatttcaaataacacacgggaaagctgagggtgacgctcttgtcgtgatggccagtgctgg
caaagactttgagctgcgcctgcctgtggacgctcggcaacagaccacttcgacaggcgagtcggctgaccccgtgactgccaccgt
tgagaattacggcggcgagacacaggtccagaggcgccaccacacagacgtctcattcatattggacagatttgtgaaagtcacacca
aaagactcaataaatgtattggacctgatgcagaccccctcccacaccctagtaggggcgctcctccgcactgccacttactatttcgct
gatctagaggtggcagtgaaacacgagggggaccttacctgggtgccaaatggagcacctgaagcagccttggacaacaccaccaa
cccaacggcgtaccataaggcgccgcttacccggcttgcattgccctacacggcaccacaccgtgttttggccaccgtttacaacggg
aactgcaaatacgccgggggctcactgcccaacgtgagaggcgatctccaagtgctggctccgaaggcagcgaggccgctgcctac
ttctttcaactacggtgccatcaaagccactcgggtgacagaactgctgtaccgcatgaagagggccgaggcgtactgtcctcggccc
ctcttggctgttcacccgagtgcggccagacacaaacagaaaatagtggcgcctgtaaagcagtccttgaactttgatctgctcaagttg
gcaggggacgtggagtccaaccctgggcccttcttcttctctgacgtcaggtcaaacttcaccaaactggtggaaaccatcaaccagat
gcaagaggacatgtcaacaaaacacggacccgactttaaccggttggtatccgcgtttgaggaattggccactggggtgaaagccatc
aggaccggcctcgacgaggccaaaccctggtacaaactcatcaagctcctgagccgcttgtcatccatggccgctgtagcagcacgg
tccaaggacccagtccttgtggccatcatgctggctgacaccggtcttgagattctggacagcacatttgtcgtgaagaaaatctccgac
cccctctccagtctctttcacgtgccggcccccgtcttcagtttcggagctccgattctgctagccgggttggtcaaggtcgcttcgagctt
cttccggtccacacccgaggatctcgagagagcagagaaacagctcaaagcacgtgacatcaatgacatcttcgccattctcaagaac
ggcgagtggctggtcaagttgatcctagccatccgcgactggattaaagcatggatcgcctcagaagagaagtttgtcaccatgacag
acttggtgcctggcatccttgaaaagcagcgggacctcaacgacccggccaagtacaaggaagccaaggaatggctcgacaacgcg
cgccaaacgtgtttgaagagcgggaacgtccacattgccaacctgtgcaaagtggtcgccccagcgccgagcaagtcgagacctga
acccgtggtcgtgtgcctccgcggcaaatccggtcagggtaagagtttccttgcgaacgtgctggcacaagccatctctacccactttac
cggcaggactgactcagtttggtactgtccgccagaccctgaccacttcgacggttacaaccagcagaccgttgttgtgatggatgattt
gggccagaatcccgacggcaaggacttcaagtacttcgctcagatggtctcgaccacggggttcatcccgcccatggcttcacttgag
gacaaaggcaagcctttcaacagcaaagtcatcattgccaccaccaacctgtactcgggcttcaccccgagaaccatggtgtgccccg
atgctctgaaccgaaggtttcactttgacattgacgtgagtgccaaggacgggtacaaaattaacaacaaattggacataatcaaagctc
tcgaggacacccacaccaaccctgtggcaatgttccaatacgactgtgcccttctcaacggcatggccgttgaaatgaagagaatgca
acaagacatgttcaaaccccagccgcctttgcagaacatataccaacttgtgcaagaggtgattgaccgggtcgagctccacgagaaa
gtgtcgagccacccgattttcaagcagatctcaattccttcccaaaagtcagtgctgtatttcctcattgagaaaggccaacacgaagcag
caattgaattctttgaggggatggtccatgactccatcaaggaagagctccgacccctcatccaacagacatcatttgtcaagcgcgcct
tcaagcgcctgaaggaaaactttgagattgttgccctatgtttgactctcatggcaaacatagtgatcatgatccgcgaaactcgcaagag
acagcagatggtggatgatgcagtgaatgagtacatcgagaaagcaaacatcaccacagatgacaagactcttgacgaggcggaaa
agaaccctctagagactagcggtgccagcactgttggtttcagagagagaactctcccgggacacaaggtgggtgatgacgtgaactc
cgagcccgcccaccccggggatgagcaaccacaagctgaaggaccctacgccggaccactcgaccgtcagagacctctgaaagtg
agagccaagctgccacagcaggagggaccttacgccggtccgatggagagacagaaaccactgaaagtgaaagcgaaagccccg
gtcgtgaaggaaggaccttacgagggaccggtgaagaagcctgtcgctttgaaagtgaaagctaggaacttgatcgtcaccgagagt
ggtgcccccccgaccgacttgcaaaagatggtcatgggtaacaccaagcccgttgagctcatactcgacgggaagacagtagccatc
tgctgtgctactggagtatttggcactgcctacctcgtgcctcgtcatcttttcgctgagaagtacgacaagatcatgttggacggtagaac
catgatagacagtgactacagagtgtttgagtttgagatcaaagtaaaaggacaggacatgctctcagacgctgcgctcatggtgttgca
ccgtgggaaccgcgtgagagacatcacgaaacactttcgtgacacagcaagaatgaagaaaggtacccccgtcgttggtgtgatcaa
caacgctgacgtcgggagactgattttctcaggtgaggccctcacctacaaggacattgtagtgtgcatggatggagacaccatgccg
ggcctatttgcctacaaagccgccaccaaggctggctactgcgggggagccgtccttgctaaggatggagctgacacattcatcgttg
gcactcactctgcaggtggcaatggagttgggtactgctcatgcgtatctagatccatgctccaaaaaatgaaggcacacatcgaccct
gaaccacaccacgaggggttgatcgtagacaccagagatgtggaagagcgcgtgcacgtcatgcgcaaaaccaagcttgcacctac
cgtggcacacggtgtgttcaaccctgagtacggccccgctgccttgtccaacaaggacccgcggctgaatgagggagttgtcctcgat
gaggtcatcttctccaaacacaagggggacacaaagatgtcaccggaagacaaagcgctgttccgccgctgcgctgccgactacgc
gtcgcgtcttcacagtgtgctgggtacagcaaatgccccattgagcatctacgaggccattaaaggcgttgacggactcgacgccatg
gaaccagacacagcgcctggccttccctgggcactccaggggaaacgccgcggcgcgctgattgacttcgagaacggcactgtcgg
acccgaagtccaggctgccctggagctcatggagaaaagagaatacaagtttgcctgtcagaccttcctgaaggacgaaattcgcccg
atggaaaaagtacgtgccggcaagacgcgcatcgtcgatgttttgcctgttgaacacattctttacaccaggatgatgattggcagatttt
gtgctcaaatgcactcaaacaacggaccgcaaattggatcagcggtcggttgtaatcctgatgttgattggcaaagatttggcacacact
tcgcccaatacagaaacgtgtgggatgtggactattcggcctttgatgctaaccactgtagtgatgcaatgagcatcatgtttgaggaggt
gtttcgcacagactttggtttccacccgaatgctgagtggattctgaagaccctcgtgaacacggaacacgcctatgagaacaaacgca
ttacagttgaaggtggaatgccgtccggctgttccgcaaccagcatcatcaacacaattctgaacaacatctacgtgctctacgcgctgc
gtagacactatgagggagttgagctggacacttacaccatgatctcctacggagacgacatcgtggttgctagtgattatgacttggactt
tgaggctctcaagccccactttaaatctcttggtcaaaccattactccagctgacaaaagcgacaaaggttttgttcttggtcactccattac
cgatgtcactttcctcaaaagacacttccacatggattatggaactgggttttacaaacctgtgatggcttcgaagaccctcgaggctatcc
tctcctttgcacgccgtgggaccatacaggagaagttgatctccgtggcagggctcgccgtccactctggacctgacgagtaccggcg
tctcttcgagcccttccagggtctctttgagattccaagctacagatcactttacctgcgttgggtgaacgccgtgtgcggtggcgcataa;
SEQ ID NO: 324:
ttgaaagggggcgctagggtctcacccctaacatgccaacgacagctcctgcgtcgcactccacacttacgtctgtgcgcgcgcggg
aaccgatggactttcgttcacccacctgcagctggactcacggcaccgcgtggccattttagctggactgagcggacgaacgtcgcttg
cgcacctcgcgtgatcgactagtactctcaacactccgcctattcggtcgttagcgctgtcctgggcactcctgctgggggccgttcgac
gctctacggtttccccccccgcgacaaactacggtgatggggccgcttcgtgcgagccgatcgcctggtgtgtttcggttgtcactcga
aacccacctttcacccccccccccccccccctaagtactaccgtcgctcccgacgttaaagggaggtaaccacaagatttgcgccttctt
gtccgaagttagagggctgtaaccgcaaactttgaaccgcctttcccagcgttaacgggatgtaatcacaagatggaccttcatccgga
agtaaaacggcaacttacacagttttgcccgttttcatgagaaatgggacgtcagcgcacgaaacgcgcagtcgcttgaggaggacttg
tacaaacacgtctcacacaggtacccacaaccgacacaaaacgtgcaacttgaaatcccgcctggtctttccaggtctagaggggtga
cactttgtactgtgattgactccacgctcggcccactggcgagtgttagtagtagtactgttgcttcgtagcggagcatggtggccgtggg
actccctccttggtaacaaggacccacggggccgaaagccacgtctcaggacccaccatgtgtgcaaccccagcacggcaactttac
cacgaaaaccattttaaggtgacactgaaactggtactcaaccactggtgacaggctaaggatgcccttcaggtaccccgaggtaaca
cgcgacactcaggatctgagaaggggattggggcttctgtaaaagcgcccagtttaaaaagcttctatgcctgaataggcgaccggag
gccggcgcctttccttaactattactgcttac;
SEQ ID NO: 325:
atgagtcttctaaccgaggtcgaaacgtatgttctctctattgttccgtcaggccccctcaaagccgagatagcgcagagacttgaagat
gtttttgcagggaaaaacaccgatctagaggcactcatggaatggctaaagacaagaccaatcctgtcacctctgactaaggggatttta
ggatttgtattcacgctcaccgtgcccagtgagcgaggactgcagcgtagacgctttgtccagaatgccctcaatgggaatggtgaccc
gaacaacatggacaaagcagtcaaactgtacagaaaacttaaaagggaaataacatttcacggggctaaagaagtagcgctcagttac
tctgctggtgcacttgccagttgtatgggcctcatttacaacagaatggggactgtcaccactgaggggcatttggcctagtatgcgcaa
cctgtgaacagattgccgattcccagcatcgatctcatagacaaatggagacaacaaccaatccactaattaggcatgagaacagaatg
gtattagccagcacgacagctaaagccatggaacaaatggctggatcaagtgaacaagcagcagaggctatggaaattgccagccaa
gctaggcaaatggtacaggcaatgagaacaattgggactcatcctagttccagcgctggtctaaaagatgatcttcttgaaaatttacag
gcctatcagaaacgaatgggagtgcagatgcaacggttcaagtga;
SEQ ID NO: 326:
atgaaggcaatactagtagtcttgctatatgcatttacaaccgcaaatgcagacacattatgtataggttaccatgcaaataattcaactga
cactgttgacacagtactagaaaagaatgtaacagtaacacactctgttaaccttttagaagacagacataacgggaaactatgtaaacta
aaaggggtagccccattgcatttgggtaaatgtaacattgctggatggctactgggaaacccagagtgtgaattactattcacagcaagc
tcatggtcttacattgtggaaacttctaattcagacaatgggacatgttacccaggagatttcatcaattatgaagagctaagagagcagtt
gagctcagtgtcatcatttgaaagatttgagatattccccaaggcaagttcatggcccaatcatgaaacgaacagaggtgtgacatcagc
atgtccttattctggagcaaacagcttctacagaaacttaatatggctggtaaaaaaaggaaattcatacccaaaactcagcaaatcctata
ttaacaataaggggaaggaagttcttgttctatggggcattcaccatccacctaccagtactgaccaacaaagtctctaccagaatgcag
atgcctatgtttttgtggggtcatcaaaatacaacaagaaattcatgccagaaatagcaacaagacccaaagtgagaggtcaagcaggg
agaatgaactattactggacactaattgagcctggagacacaataacattcgaagcaactggaaatctagtggcaccaagatatgccttc
gcaatggaaagaggctctggatctggtattataatttcagatacaccagtctacgattgtaatacggcttgtcaaacacccaaaggtgcta
taaacaccagtcttccatttcaaaatatacatccagtcacaattggagaatgtccaaaatatgtcaaaagcacaaaattgagaatggctac
aggattaaggaacatcccgtctattcaatctagaggcctgtttggagccattgctggctttattgagggggggtggacaggaatgataga
tggatggtatggttatcatcaccaaaatgagcaggggtcaggatatgcaccagaccgaaagagcacacagaatgccatagacgggat
cactaacaaagtaaactctgttattgaaaagatgaacacacaattcacagcagtgggtaaagaattcaatcacttggagaaaagaataga
gaatttaaacaaaaaggttgatgatggttttctgaatgtttggacttacaatgccgaactgttggttctattggaaaatgaaagaactttggat
tatcacgattcaaatgtgaagaacctatatgagaaagtaagaagccaactaaaaaacaatgccaaggaaattggaaatggctgctttga
attttaccacaaatgtgatgacacgtgcatggagagcgtcaaaaatgggacttatgattatccaaaatactcagaagaagcaagactaaa
cagagaagagatagatggggtaaagctggaatcaacaaggatttaccagattttggcgatctattcaactatcgccagttcattggtactg
ttagtctccctgggggcaatcagtttctggatgtgctccaatgggtctttacagtgcagaatatgtatttaa;
SEQ ID NO: 327:
atgaagactatcattgctttgagctacattttatgcctggttttcgctcaaaaacctcccggaaatgacaacagcacggcaacgctgtgcct
ggggcaccatgcagtgccaaacggaacgctagtgaaaacaatcacgaatgaccaaattgaagtaactaatgctactgagctggttcag
agttcctcaacaggtaaaatatgcgacagtcctcaccaaatccttgatggagaaaactgcacactaatagatgctctattgggagaccct
cattgtgatggcttccaaaataaggaatgggacctttttgttgaacgcagcaaagcctacagcaactgttacccttatgatgtgccggatta
tgtctcccttaggtcactagttgcctcatcaggcatgctggagtttaacaatgaaagcttcaattggactggagtcgctcagaatggaaca
agctctgcttgcaaaaggagatctgataaaagtttctttagtagattgaattggttgcaccaattaaaatacaaatatccagcactgaacgt
gaccatgccaaacaatgaaaaatttgacaaattgtacatttggggggttcaccacccgggtacagacagtgaccaaatcagcctatatg
ctcaagcatcagggagagtcacagtctctaccaaaagaagccaacaaactgtaatcccgaatatcggatctagaccctgggtaagggg
tgtctccagcagaataagcatctattggacaatagtaaaaccgggagacatacttttgattaacagcacagggaatctaattgctcctcgg
ggttacttcaaaatacgaagtgggaaaagctcaataatgaggtcagatgcacccattggcaaatgcaattctgaatgcatcactccaaat
ggaagcattcccaatgacaaaccatttcaaaatgtaaacaggatcacatatggggcctgtcccagatatgttaagcaaaacactctgaaa
ttggcaacagggatgcggaatgtgccagagaaacaaactagaggcatattcggtgcaatcgcgggcttcatagaaaatggttgggag
ggaatgatggacggttggtacggtttcaggcatcaaaattctgagggcacagggcaagcagcagatcttaaaagcactcaagcagca
atcaaccaaatcaacgggaaactgaataggttaatcgagaaaacgaacgagaaattccatcaaattgaaaaagaattctcagaagtaga
agggagaattcaggacctcgagaaatatgttgaggacactaaaatagatctctggtcgtacaatgcggagcttcttgttgccctagagaa
ccaacatacaattgatctaactgactcagaaatgaacaaactgtttgaaagaacaaagaagcaactgagggaaaatgctgaggatatgg
gcaatggttgtttcaaaatataccacaaatgtgacaatgcctgcatagggtcaatcagaaatggaacttatgaccatgatgtatacagaga
cgaagcattgaacaaccggttccagatcaaaggtgttgagctgaagtcaggatacaaagattggatcctatggatttcctttgccatatca
tgttttttgctttgtattgttttgctggggttcatcatgtgggcctgccaaaaaggcaacattaggtgcaacatttgcatttga;
SEQ ID NO: 328:
atgaataaaatatttagagttatttggagtcatgctcaacaggcttgggggttgtatctgagttagtaaagtctcataccaaaacatccgctt
acacggataaaagagctcaagtatgcacctcagattattttttagataaacagcaagataaatttaaattaagtcttttaagtctagtattacta
ggtatattttttagttcagtaggttcagctgcatatcttcaagatggtgctaatgagggatcgaatataggaactgacgacggtactattggt
attggtcaagagagtagggcttcgtatggtgctgttgctatcggtcagaaggcaaaagctgaagctaggcataatattgcgataggttat
ggagcagattcaggaacacaagtaaactctcttgcgataggctaccgcgctacagtaagtgggactggagcaattgccttaggcaaac
aagccaattcgacaaatagtcaaacaattgccatcgggagtgattcaaaagcgagcggagatgaatctattgccttaggcggacaagc
caattcgacaaataatcaaacaattgccatcgggagtcagtcaaaagcgagcggagaacaatctattgtcttaggcacaggggccagt
gtgacaggtactcaaacaattgtaataggcgcacgtgctagtgcaagtgggcaccaatctgttgctatcggggcaaatacccaagcgc
aggggtatggctccatatcaatcggtggagatgatttagctacaacgaaatatcaagatgatgctcaagactattccagcacaacaactg
cggcaggtcacgcctctgttgctattgggggtcgatctaaggctcagggcgatggatctattgttgtaggtcctttagcatatgcaaggta
tgctgaaggcattgctatcggtgcgagaagtaagtctaacaatgagtacggtattgcggttggtagtggtgcatttgctggaaagcactc
cattgctgttggtaaaagtgctactgcaagtcaaacaggagcttccgcattcggtgaaagcgcacgagcaatcggacagtatacaaca
gcattggggagttatgcggaagcagaaacgcaagatggcgttgctctaggttatcgatccaaaactagtcgtcggtcagggcaaagag
gttggacaccagagaatacaaattattctattaacggaagtacattatctgctacgcatgcagcggtagcagtaggtgatggcaccaccg
tcactcgtcaaattaccagtgttgctgcagggacagcagatactgacgcagcaaacgtggcgcaattaaaagcactgactttaaagatc
agtggcgatagaaatacgcagggtcacacaacattttataacgaaacgctctcaattgtaggggctgatggtattagtacggcagttgag
cagggaaatggcaattcaaaaattaccattactggctcaaaaacgtattttcatactaactataacgaacagtctcaaggtcggggagatc
ctacgactaatttcggcacaattactgacaaagctggagctacaggaacctatgcaataacagcgggggtaaatgcctctgcggctgg
aaattatggtattgcgatggggtataagagtaatgctagtgcttatgctgttgctcttggtagtgaatcgaaaggtgctggggcagattcca
ttgcgataggtaatcttgcgagaacaacaggggtagactctgttgttgtgggtgcccatatcaatgtgacaggtcaaaaatcagtggcag
ttggacgccaagcaaatgctagagattattctactgccttgggttatatggcctctgccagtggtacgtactctgttgctgtgggtgaaaat
gccacgataaatgtaaatgctgctagagcaaccgcacttggtcataataccgttgtcaccgtgggtggcggtgtggcattaggttatgga
tctaatgcaaatatagctggcggtgtagaggggttaaaacaagctcattctgtcacaacgggaacaagcactgaagctaacggctttaa
atccacacaaaaggttgatggtaataatattggtgcagtttctgtgggtggtacaaccatcaaacgccaaatcgtcaatgtggcggctggt
acacaagataccgatgcggtaaacgtggcacagcttaaatctttgacgatgaaaattggtggtaataccaatgctaatacacagccaaa
agtggggttgtgggatggtattcttaaagtgctaggcacaaatggcgaaatcaagaccaatgcgtctggctcaaccattacaatatcact
agacgatacgattaaaaataaattagctgatgccagagcaggaagtttgatattcaagggcgaaaaaacagataacggtacaacaaat
gatgtttcgggtcaaaaatggaaggccaacgaagataagaccgttaccattacaagtaacgaaacataccaaaacggtggtgttcgata
caaaggcgataacattgaaatttatcgtagaaatctagaattccatgtgttgatgaaagagacaccaacattcagcagcgttcaatatggc
gataatggacctaagattaccagcaccactgacggtaatctaaaagtaacaggtacagacggcacttcctcagttaagatcaccaattta
gcacgaggtacacaaaatagcgatgcggtgaactacatgcaattttcaaatgctggttggaaacttgcaattgctccaggaacggggg
gtcaagcaactccacctggggcacatcttatcaaaataaacgataccgtaacctttaccgctggaaataatattaaattagaacaagcgg
gcggaaatattacgatttctacgattggtaagttaattaaaacgactgaaagcctagaaaatggcgatctaaaaattatctatacagataatt
cgcataacattatcaagaaaggtgaaaaaggagatcgtggcgagcgaggtctaagaggtgaaacaggccctgcgggtccgattggtc
cagtgggtccagcaggggctaggggtgagcgaggccctgcaggtgtagctggtcctaagggtgagaaaggtgatccaggaccaga
gggccctcaaggtcctagaggtgagcaaggtctaagaggtgaacgaggcctagcgggtcctagaggcgaaactggtccgaggggt
ccagtgggaccacaaggagcgcaaggaatgccgggtgctcagggacagaagggtgaccgaggagaaactggccctgcgggtcct
agaggcgaagctggtcctgctggagcaacaggaccacaaggtgaaaaaggagatcaaggtccgatgggtccggcaggcccagcg
ggagagcgaggcgagcggggtcctaaaggagatagaggtccaaaaggtgatacaggtgagagaggtgcaactggccctgcgggt
ccgatgggtccagcaggcccagcgggagagcgaggagaaactggacctgcaggtgtacctggtcctaggggtccagagggtccta
gaggcgaaactggtttaacaggtccgaggggtccagtgggaccacaaggaccgcaaggaacaccgggtactccgggacagaagg
gggataaaggcgatccaggacaagcgggtccagcaggacctaggggtccagtgggtccgaaaggtgaaacaggagctcaaggtc
cgataggtccggcaggcccagcgggagagcgaggcgagcagggtcctagaggcgaagctggtccaacaggtccaacaggtccg
aggggtccagtgggaccacaaggaccgcagggaacagcgggtgctcagggacctaagggtgagcgaggccctgcaggtgcagct
ggccctgcgggtccagtaggtccagcgggaccacaaggtgaaaaaggagatcaaggtccgatgggtccggcaggcccagcgggt
gctcagggcatacaaggtccaaaaggtgatagaggcgaacaagggcttccaggggtagcaggtcctaagggcgatagaggcgaag
ctggtccagtgggtccagcaggaccagcgggagagcgaggcgagcagggtcctaggggggaacaaggtgcaactggccctgcg
ggtccaacggggcctaggggtgaaccgggtccaacgggaccacaaggaccgcaaggaacaccgggtactccgggacagaaggg
ggataaaggcgatccaggacaagcgggtccagcaggacctaggggtccagtgggtccgaaaggtgaaacaggagctcaaggtcc
gataggtccggcaggcccagcgggagagcgaggcgagcagggtcctagaggcgaagctggtccaacaggtccaacaggtccga
ggggtccagtgggaccacaaggaccgcagggaacagcgggtgctcagggacctaagggtgagcgaggccctgcaggtgcagct
ggccctgcgggtccagtaggtccagcgggaccacaaggtgaaaaaggagatcaaggtccgatgggtccggcaggcccagcgggt
gctcagggcatacaaggtccaaaaggtgatagaggcgaacaagggcttccaggggtagcaggtcctaagggcgatagaggcgaag
ctggtccagtgggtccagtaggaccacgaggaccacagggaacagcgggtgctcagggacctaagggtgagcgaggccctgcag
gtgaaactggacctaagggtgagaaaggtgatccaggaccaaaaggcgaaactggtccaacaggtccagtgggtccagcaggacc
agcgggagagcgaggcgagcagggtcctaggggggaacaaggtgcaactggccctgcgggtccaacggggcctaggggtgaac
cgggtccaacgggaccacaaggaccgcaaggaacaccgggtactccgggacagaagggggataaaggcgatccaggacaagcg
ggtccagcaggaccacgaggccctgcaggtgcagctggacctgcgggcccagcagggcctaggggtgaccgaggagaaactggt
cctgcgggtccaacaggggctaagggtgaacagggtcaaaaaggagatacaggtccaatggggcctgctggtccaaaaggtgatgc
aggtcctagaggcgaagctggtcctgctggagcaacaggaccacaaggtccaaaaggagataatggagctacaggtcctagggga
gagaaaggtgaacctggccctgcgggtccgattggtccagtgggtccagcaggggctgctggtccagcaggcccagcgggagagc
gaggccctacgggtcctaaaggagatgcaggtccaaaaggagatacaggtcagaaaggagaaactggccctgcgggtccagcagg
ggctaagggtgaaccgggtcctagaggtgagcaaggtattcaaggacctacgggtccaacgggaccacaaggaccgcagggaaca
gcgggtattcagggacctaagggtgagcgaggaaatgtgagtgtcagcggtttaccgatcgagtatgcaacggaagacggcaaatca
attatcaatatgggcggtaatttctatttggaagaacctgctaaagatggttcgattaagttaattccagtggtgaatgttaaaggtaaattct
caaccaaaacgcaaaatccagatggcagtattacgcttaagtcattagcagtaaaagtgaatttggcaaatgaaactccgatggtattag
gtaatgtcgctgaaggggtagcagatacggacgctgttaatgtgaaacagttgaaatctgcgaaaactgaagtggaatctaccgatcac
agtgtggtgattaaagagcgtcagggcgataatcagcaaatcgtgtatgatttggcggttgctaaaacgaaactcactgcctctaaggat
aaacgcaccattagtgcagcagataaaggcaaccattttgcgacaggagatgaagtcgcagtagcaattaataccgcaacagcagcc
gcaagaaccgaagttgaagcgggtaaaaatgtgaaagtgacttcaaaaacgggggcaaatggtcagaatatttacaatgtgagcgtgt
ttggagatttaagcgacattacttcaattagtaatggcgatacgaaagtatctttaggtaaagataagcaaggaaatccagttgtaaatatg
aatggtgccagaattaccaacgttggagatggtagtgctgagggcgatattgtgaatgttcgtcagctcaacaaagtggtttcttctgtga
atacaggatttaatcaattatcaagagatattgttaatgcaagagcgggtattgcttctgctggggcgatggctaatttgccacaaatttcttt
accaggtaaaagtgctatttctgtttctaatgcacaatatcgcgggcaatctgcctatgctataggttattccaaaatttctgataatggcaaa
tggcttattcgagcgtctgttagcagtaatactcagcgggatactatgattggaggaggggtaggttttgtgtggtaa;
SEQ ID NO: 329:
aagcgaaagtagcagcaggtgaagctggtggtattactcagcatatcggtgcatatcacgttgaaaccgacgacggtaagatgattacc
ttcttagatacaccaggacacgcggcatttacctcaatgcgtgcgcgtggtgcgaaagcaacggatatcgttgttcttgtagtagcagct
gacgatggcgtaatgccacaaaccattgaagcaatccaacacgcgaaagcagctggtgcgccgatcgtggttgcggtaaacaaaatt
gataaaccagaagcaaacctagagcgtgtagaacaagagttattacaacacgaagtgatttctgagaaattcggtggtgatgttcaattt
gttcctgtttcagcgaaaaaaggaatggggattgacgacttacttgaagccattcttcttcaatcggaagtattagaattaagtgcggtaaa
agagggtatggcaagcggtgtggttatcgaatcttacctcgataaaggtcgtg;
SEQ ID NO: 330:
atgaaacaaatcgttttaaaaacaagcttattgatgaccctctcttcattattagttgcatgtagcggcggtggcggtagcgctggaaatcg
tgctgaccgtgtagaggaaaaagcacaaccggttcaatcaaatagtgagccttcttccgctccaatcaaaaatcctactaataccgctac
gaatgattctcttcatgacaaactttcaatgtcttctcatgacacatccaaagaaaatagtcaacaatcctcctttaaagcccctctagaaca
agaaaaaaaccaacctgcacaagaaaatctcacttggacaggttatcatgtttcagaagtgggaaatgcgagtaataatgtagataaag
ataacgttacggtattcactttcgtaaaatataattctcaatacaatgatgatccagtttttgataaaacaaaaacacaaagtaaaacaatatc
attagttgacggaaaaaatgagaataaagaggattattataactttacgttaaaagacgctttattttattatggaagttatggacaaccttca
gcagattacaaaaaagtagaaaaaaattatatttatgcaattaaaccagatgcaataaataatgagaacctcaatgcactaactgcaactt
attatcaagaagatggttttatatattccgtattaagtgatgtaaatcgagttggttcagaatatattcctcagtatggcaatgtgactcttacttt
ccgaaatggcaagatttatggtgaaatctacagatataatagaggacgtgatgatttgtttcagctctcaggagaaggacaaaacttaact
ataacaccacacaaggacaatccccataaactatcccctacaggacccgacaacatggcaatggagctgaattttatcaacgcagaaa
aaactgataaaaaatacgttgttggtgtaggaaaagctgaaaaatattatgggttattatttgctgaaaaaagtcaccaagcacaataa;
SEQ ID NO: 331:
atgcattcaagtaaaccttatttttggggaagtgtgtttcttttgttcacgtttttaggtgcctatcttgcttcggcaatagatcgtgattttggtc
gaattgaggtgagccatgtgagctttatgacagaagaaatgcaaccgatggtcgcgaagctatatcgtccagtttctgctacggcagac
acaccgaagccaggacttttggcattgcatggctatcaaagtgataaagaagcaactagtacatttggtgcgttggagctggctaagcg
cggttttgtggtgctagcgatcgatcattttggacatggctattctactaagctacccgcttcaaataaaaatatgagtggagcgaataatg
gttatcaatatttaaaaacgttgccgtttgtagataacacacggttaggtctctttggtcactctactggcgcgttaaatgcaattcgcgtggc
aaaactcaatccagatcacaaagctgtcaatggattaagtagcaatggtggtgagatgacattgaataactacttattgacacaaggattg
tacgaagaaattggtggctatcgtgaaagaaccttccctgtaaaaagtttgattcatcatcctaaccgcctgaaagcatttgggttaggcg
aaaatgaaactttgcagtgggatcatacctatggtgactttaacaccggttctgcacgtcgtgcggcaatggtcgatggtacccatcttgg
ggtgatgattgcttcccagagtaataaagaagccattctttggtttaatcaagcattgcaacatggcgaaaaaggtgcggattggattgat
cctgaccaacagacctattggtataaagaacttacagggttatttgccttagcttgtgctttgcttgccacattatgttttgctagcggactttt
aaaaacgacttattttggcgtgatgaatcaagcggtcacagaaaagacagcaatttcgagcaaacaatggtggggctttacgctgattaa
tattgtgctgacattactgctctatccgctatttacccagtggggcggtgcgaatgaaccgattgcagcgaaattgagttttatgccacttga
aatgggaaatggcattattttatggctcgttgtgagtgggcttgtcggtagtcttttatttggcttgtggcaaagaaaagcacagttttgttgg
gcggagtttggtgtgttgagccaatctgcttccttgacaacggcgcaactgattggacgttatttattactcagcttattgttatttgccggttt
atatttccttgtcaatctgatttatcaatatttccatgttgagttacgtttcttatggccattattgaagccattaacgacagagcggtttaattta
tttatcgtgtattggttacctattttggtctttttcttcgtgttcaacggtttgatcgtgtcagtccaaatgaaacaaaaagtagcgagttcgtttac
ggcaacattgctgatctggagtttcaaaaccgcactttttgctactggtggtttaatcattttatggttattccattttgttcccggttttatgcaa
atcggtccgggatttgatgtggtaggactgccacaatttggtggacgttggatgatgatgttagccgtcattattccacagtttattgtcttca
ccgtgatcaatcactggtgctatttaaaaacaggctatatttatttaggggtatttttcacctctctgttaatgacttgggtgttagtcggaggt
caagtcattgggcgattcttagcctaa;
SEQ ID NO: 332:
atgaatattgcaacaaaattaatggctagcttagtcgctagtgtagtgcttaccgcatgtagtggcggcggctcatcgggttcatcgtctaa
accaaattcggaacttacacctaaggttgatatgtccgcaccaaaagcggagcagccaaaaaaagaggaagttccacaagcggataa
ttcgaaagcggaagaaccaaaagagatggctccgcaagtagatagcccgaaagcggaagaaccaaaaaatatggctccacaaatgg
gtaatccaaaactaaatgacccacaagtaatggctccgaaaatggataatccgcaaaaagatgccccaaaaggagaagaactaagta
aggataaaagtaatgcggaaattcttaaggaattaggggttaaggatattaattcaggtatcattaataatgctgatgtagttctgaatttaaa
aatagatgaaaaagatcacattacagtcgtattagataagggtaagattaatcgtaatcatctaaaagtaactaatacaatttctgctcaaga
cattaaaaccttaaaagattcttcaggcaaattgttgggttactatggatatatgcagttaaatcaagttcgacaagatgaaaattatagcga
tgaaaaagttagtttgaatgaatattatttattatcaatgaacgatgccgataaaatacgtccgactaaatctatatcatataagggagacat
gttttatagttacaaagatgtaggaaatcagaaattaaaggcttctgtagaagcttcttatgatgatgtaacaaaaaaagtatcaatgaaagt
atttggtgagaataatgattactggaaattaggtgagtttggtagaactaatttattagaaaatcaagtgactggagcaaaagttggcgaag
atggtaccattataaatggaactttatattctaaaatagataattttcctttaaaactaactcctgacgcaaacttctctgggggtattttcggta
aaaatggcgaagtattagccggaagtgctattagtgaaaaatggcaaggcgtaatcggtgctacggcaaccacaaaagaagataaaa
aataa;
SEQ ID NO: 333:
atgacaaaattaactatgcaagatgtgaccaatttatatttatataaaacgaaaactctacctaaagatagattggatgattcacttatttctga
aataggaaaaggagatgatgatattgatagaaaagaatttatggggggccgggacgttttgtgaccgctgataactttagcgttgtaag
agatttttttaatgctgggaaatcacgcattattgcgccgcaagtcccgcctattcgttcacagcaggaaaaaatcttggtcggtttaaaac
cgggcaaatattccaaagcgcagatattggaaatgctgggttatacgaaaggcggagaagtggtaaatggcatgtttgccggtgaagt
ccagacattaggcttttatgacgatggcaaaggggatttactcgaacgcgcctatatctggaataccacaggatttaaaatgagcgacaa
tgccttttttgttatagaagaatcaggcaaacgctatattgaaaactttggtattgaacctcttggtaagcaagaagattttgattttgtcggcg
gcttttggtctaacttagtgaatcgtggtttggaaagtattatcgacccatccggtatcggtggaacggtaaaccttaactttaccggcgag
gtggaaacctacacgttagacgaaacaaggtttaaagcggaagcggcgaagaaaagccattggagtttagtgaatgcggcgaaagta
tacggcggtttagaccaaattattaaaaaactatgggacagtggctcaattaagcatttatatcaagataaagatacgggcaaattaaaac
cgattatttacggcacggccggcaacgacagtaagattgaaggcactaaaatcacccgtaggattgcgggtaaagaagttacgcttgat
attgccaatcagaaaattgaaaaaggcgtgttagagaaattggggctgtctgttagtggttcggatatcattaaattgttgtttggagcattg
actccaactttaaatagaatgttgctatcacaacttatccagtctttttccgatagcttggctaaacttgataatcccttagccccttacactaa
aaatggcgtggtttatgtcaccggcaaagggaatgatgtgcttaaaggaactgaacatgaggatttgtttctcggtggtgaggggaatga
tacttattatgcgagagtaggcgatacaattgaagacgccgacggcaaaggtaaagtctattttgtgagagaaaaaggggtacctaagg
cggatcctaagcgggtagagtttagcgagtacataacgaaagaagaaataaaagaggttgaaaaggggttattaacttacgcagtttta
gaaaattataattgggaagagaaaacggcgactttcgctcatgcgactatgcttaatgagctttttactgattatactaattatcgttatgaag
ttaaaggactaaaattgcccgccgttaaaaagttaaaaagtccgttggtggagtttacagctgatttattaactgttacgcctattgacgaaa
acggaaaagcacttagcgaaaaaagtattacggttaaaaattttaaaaatggtgatttaggaataaggttgttggatcctaatagctattatt
atttccttgaaggccaagatacgggtttttatggtcctgctttttatattgaacgaaaaaacggtggcggcgctaaaaataactcgtcggga
gcaggaaatagcaaagattggggcgggaacgggcatggaaatcaccgaaataatgcctccgacctgaataaaccggacggaaataa
tgggaataaccaaaataacggaagcaatcaagataatcatagcgatgtgaatgcgccaaataacccgggacgtaactatgatatttacg
atcctttagctttagatttagatggagatgggcttgaaaccgtgtcgatgaacgggcgacaaggcgcgttattcgatcatgaaggaaaag
gtattcgtaccgcaacgggctggctcgctgcggatgacggttttttagtgttagatcgtaaccaagacggcattattaatgatataagcga
gttatttagtaataaaaatcaactttccgacggcagtatttctgcacacggttttgcgacattagccgatttggatacaaaccaagatcagc
gtatcgaccaaaatgataagctgttttctaaactccaaatttggcgggatttaaatcaaaacggttttagtgaagcgaatgagctgtttagct
tagaaagtttgaatattaaatctttacataccgcctatgaagagcgtaatgattttctagcgggcaataatatccttgctcagcttgggaagt
atgaaaaaacggacggtacttttgcacaaatgggcgatttaaatttcagttttaacccgttttatagccgatttaccgaagcgttaaatttaac
cgagcaacaacgtcgcacaattaatctaaccggcaccggtcgggttcgggatttgcgtgaagccgccgcactttctgaggagttggct
gctttattacaacagtacactaaggcctccgattttcaggcacaacgagaattattgcctgccattttagataaatgggcggcaacggattt
acagtatcaacattatgataaaacattacttaaaacggtagaaagtaccgatagtagtgcttctgtcgttagagtcacgccttctcaattaag
tagtatacgcaatgcaaagcatgatcctaccgttatgcaaaactttgaacagagtaaggcaaaaattgcgactttaaattcgctctacggg
ttaaatatcgatcaactttattacacgacggataaagacattcgctatattactgataaagtgaataatatgtatcaaacaaccgtagaactt
gcctaccgttctttacttttacaaacgcgtttgaagaaatatgtttatagcgttaatgcgaaacaattcgaagggaaatgggtaaccgattat
tctcgtactgaagccttatttaactctacttttaaacaatcgcctgaaaatgcattatatgatttaagcgaatacctttctttctttaacgatccta
cggaatggaaagaagggctattactgttaagccgttatatagattatgctaaagcacaaggattttatgaaaactgggcggctacttctaa
cttaactattgcccgtttaagagaggctggagtaatttttgcagaatcgacggatttaaaaggcgatgaaaaaaataatattttgttaggtag
ccaaaaagataataacttatcgggtagtgcaggtgatgatctacttatcggcggagagggtaatgatacgttaaaaggcagctacggtg
cagacacctatatctttagcaaaggacacggacaggatatcgtttatgaagataccaataatgataaccgcgcaagagatatcgacacct
taaaatttaccgatgtgaattatgcggaagtgaagtttcgacgagtagataatgacttaatgttattcggttatcatgatacggattcggtca
cggtaaaatccttctacagccatgtagattatcaatttgacaaattggagtttgctgaccgcagtataactcgcgatgaactgattaaagca
gggcttcatctatacggcaccgatggcaatgatgatataaaggatcatgcggattgggacagcattttggaaggcggcaaaggcaacg
atattctaagaggtggctacggtgcggacacctatatctttagcaaaggacacggacaggatatcgtttatgaagataccaataatgataa
ccgcgcaagagatatcgacaccttaaaatttactgatgtgaattatgcggaagtgaaattccgacgagtagataatgacttaatgttattcg
gttatcatgatacggattcggtcacgataaaatccttctacaaccatgtagattatcaatttgacaaattggaatttgctgaccgcagtataa
ctcgtgatgaactaggtaaacaaggtatggcattatttggcactgacggtgatgataatatcaacgactggggacgtaactcggtgattg
atgccggtgcgggtaatgatacggttaatggcggtaatggcgatgacaccctcatcggcggcaaaggtaatgatattctaagaggtgg
ctacggtgcggacacctatatctttagcaaaggacacggacaggatatcgtttatgaagataccaataatgataaccgcgcaagagatat
cgacaccttaaaatttaccgatgtgaattatgcggaagtgaaattccgacgagtagataatgacttaatgttattcggttatcatgatacgga
ttcggtcacggtaaaatccttctacagccatgtagattatcaatttgacaaattggagtttgctgaccgcagtataactcgcgatgaactgat
taaagcagggcttcatctatacggcaccgatggcaatgatgatataaaggatcatgcggattgggacagcattttggaaggggcaaa
ggcaacgatattctaagaggtggctacggtgcggacacctatatctttagcaaaggacacggacaggatatcgtttatgaagataccaat
aatgataaccgagcaagagatatcgacaccttaaaatttactgatgtgaattatgcggaagtgaaattccgacgagtagataatgacttaa
tgttattcggttatcatgatacggattcggtcacgataaaatccttctacaaccatgtagattatcaatttgacaaattggaatttgctgaccg
cagtataactcgtgatgaactaggtaaacaaggtatggcattatttggcactgacggtgatgataatatcaacgactggggacgtaactc
ggtgattgatgccggtgcgggtaatgatacggttaatggcggtaatggcgatgacaccctcatcggcggcaaaggtaatgatattctaa
gaggtggctacggtgcggacacctatatctttagcaaaggacacggacaggatatcgtttatgaagataccaataatgataaccgcgca
agagatatcgacaccttaaaatttactgatattaatttatccgaactttggtttagccgagaaaataacgatttgattattaaatcattattaagt
gaggataaagtcacggttcaaaattggtattcacaccaagatcataaaatagaaaatattcgtttatcgaatgagcaaacgttggtgagca
ctcaggtggagaagatggttgagtcgatggccggctttgctcagaagcacggaggagagatatctcttgtgtcgcttgaagaggtaaaa
caatatatcaatagcttaacagctgctttataa;
SEQ ID NO: 334:
atgtcaaatgccaaagcttacatccaagcttcttttgaagcagtcaaagcccgcaacccacatgaaacagaattcctccaagctgtagaa
gagctcttctctacacttgagcctgtttttgaagcacacccagaatacatcgaagaaaacatcttggctcgtatcgttgagcctgagcgtac
catcagcttccgtgttccatggacagataaagatggaaatgttcaagtcaaccgtggctaccgtgttcagttcaactcagctgtaggtcctt
ataaaggcggtcttcgcttccacccaactgtaaaccaatccatcttgaagttcctcggttttgagcaaatcttcaaaaacgtcttgactggtc
ttccaatcggcggtggtaaaggtggttcagactttgatcctaaaggaaaaactgatgctgaaatcatgcgcttctgccaaagcttcatgac
tgaattgcaaaaacacatcggaccttcacttgacgtccctgctggtgacatcggtgtcggtggtcgtgagatcggttacatgtacggtca
atacaaacgcctccgccagtttgatgcaggtgtcttgactggtaaacctcttggcttcggtggttcattgatccgcccagaagcaactggt
tacggtttggtttacttcactgataacatgttggcagcaaacggtaaatccttcaaagaccaaactgtccttatctcaggttctggtaacgtt
gcccaatatgctgttcaaaaagcgactgaacttggtgcaaaagttatttctgtttcagactcaaatggttacatcattgacgaaactggtatc
gacttcgacctcttggtggacatcaaagaaaaacgccgcgctcgtttgacagaatacgctgcagaaaaatcaactgctaagtacttcaa
aggttctgtatggaactacgatggcaaggctgatattgcccttccatgtgcgactcaaaatgagatcaacggcaaacaagctgctgccct
tgtaaaaaatggcgtgtactgtgtggctgaaggtgccaacatgccatctgaccttgatgccatcaaagtctacaaggaaaatggcgttct
ctacggactcgcaaaagctgccaacgctggtggtgtagctgtatctgcccttgaaatgagtcaaaacagccttcgcttgtcatggactcg
tgaagaagtagacggccgtcttaaagacatcatggccaacatcttcaacacagccaaagaaactgctgaaaaatacgaccttggtaca
gactaccttgcaggtgctaacatcgcagcctttgaacaaattgcggatagcatgattgcccaaggtttggtataa;
SEQ ID NO 335:
atgttactagaagtgtccattaaaaacttcgccattattgagcaggtatcactgaattttgaaaacgggatgaccatcctatctggtgaaac
aggtgcaggtaaatccattatcattgatgccatgaacctgatgctaggggcgcgtgcgacgacagatgttatcagacatggggccgcc
aaggctgagattgaagggctcttttcctttgaaaatagcagagctctcgagcaaattttgcttgaacaggggattgaagtagctgacgaa
ctcattatccgccgtgaaatcctgcaaaatggccgttcggtcagccgtgtcaatggacaaatggtcaatttatctgtcttgaaacagattgg
gcaatatttagtagatattcatggtcaacatgaccaggaagaattgatgaaatcccagcaccatatccgtcttttggacagtttcggagaa
gatgaattttggagtcttaaagaccgttatcagacaacctttgatgcctatcgtagtcttcgcaaacgagttcttgaaaagcaaaaaaatga
gcaagaacacaaggcgcggattgagatgctagaataccaaattgctgaaatcgaagcagcggatttgaagtctggggaagatattcaa
ctcaatcaggaacgcgataaactgctcaaccacaaacaaattgcagatacactgaccaatgcctatgcactgttagacaatgaagattttt
caagcttgaacaacctacgctcagctatgagtgacttgcaaagtctggaagaatttgatccagactacaaacagctctcttctagcctgac
agaagcttattatgtcgttgaagatataaccaagcgtctcagcgatgtggtcgataatctagattttgacggcaatcgtctcatgcaattgg
aaagtcgtctggatttgctcaataccattacgaagaaatatggtggaactgtcgatgatgttttggactattttagtaaaatcagcgaagaat
acaatctattgacaggcaatgatttatctggagatgatttggaagttcagcttaagaacttagagaaagaattggttgaacgagcaggtca
gctcagccaatcacgccatgaattggctgttgttttagaagatattatccgccaagaattgcaagacttgtatatggagaaagctcgtttcc
aagttcgatttactaaaggaaaatttaatcgagaaggaaatgaaacagtagaattttacatctctaccaacccaggtgaagacttcaagcc
tctggttaaagtggcttctggaggagaattgtctcgcttgatgttggccattaagtcagcctttgcccgtaaagaaggcaagacttccatcg
tctttgacgaggtggatacaggggtgtctggacgtgtggcccaggccattgcccagaaaatttataaaatcggacaatatgggcaagta
ttggcgatttctcatctaccacaagtcattgctattgcagattatcagttctttattgagaaaatatccgacgaacattcgaccgtttctcgtgt
ccgtctcttgaccaaagaagagcgtattgaggaaattgccaagatgttagccggtgacaaaatcacagatgcagcccgtaatcaagca
aaagaattagtagaaaaaggttag;
SEQ ID NO: 336:
atggtagttaaagttggtattaacggtttcggacgtatcggtcgtcttgctttccgtcgtatccaaaacgtagaaggtgttgaagttactcgt
atcaacgaccttacagatccagtaatgcttgcacacttgttgaaatatgacacaactcaaggtcgtttcgacggtactgttgaagttaaaga
cggtggtttcgaagttaacggtaaattcgttaaagtttctgcagagcgcgagccaggaaacatcgactgggctactgatggcgtagatat
cgttttggaagctacaggtttctttgcttctaaagaaaaagctgagcaacacatccacgctaacggtgctaaaaaagttgttatcactgctc
ctggtggtaacgatgttaagactgttgttttcaacactaaccacgacatccttgatggtactgaaacagttatctcaggtgcttcatgtacta
caaactgtttggcaccaatggctaaagctcttcacgatgcatttggcgttcaaaaaggtttgatgactacaatccacggttacactggtga
ccaaatggttcttgacggaccacaccgtggtggtgaccttcgtcgtgcacgtgctgctgctgcaaacatcgttcctaactcaactggtgct
gctaaagctatcggcttggtaatcccagaattgaacggtaaacttgacggtgctgcacaacgtgttccagttccaacaggttctgtaactg
aattggttgcaactcttgacaagaaagtaactgctgaagaagtaaacgctgctatgaaagctgctgctactgaatcatttggttacactga
agaccaactcgtatcttcagatatcgtaggtatctcattcggttcattgtttgatgcaactcaaactaaagttatcgaagttgatggcgagca
attggttaaagttgtttcatggtacgacaacgaaatgtcttacactgcacaacttgttcgtactcttgagtacttcgcaaaaattgctaaataat;
SEQ ID NO: 337:
atgtcaaaaaataatatagtggagtgctttatagaaattgcaaagcactccaatcttaaatatgagtgtgttgatggcaaattgaaactagat
agagtactatttggttcaatggtatatccgcacaactatggatatatttctgatactctagcagaggatggagatcctctagatgtagtagtg
ctatctaatttctctgtaactccaggaacttatttggattgcaaaattcttggttctctagaaatggtagattctggagaacaagattgaaaagt
tattgcaattatggatgcggatccaagactcaagcatataaattctctagatgatgttcctcaacattgaattgctgaattgagaaacttttttg
aaagttacaagcaactagagaataagaaagtttcccttggaaactttatctctctagaatcaactctttctttaattgaagaatcaaaagcta
gatgaagaactcaagggggagaataa;
SEQ ID NO: 338:
atgacaatccacaaagtagcaatcaatggattcggaagaatcggaagattactatttagaaatcttctttcttctcaaggagttcaagttgta
gctgttaatgacgtagttgacattaaagttcttactcaccttttggtttatgacagtgctcaaggaaaactaaaagattgagaagtaagttgtg
attcagaatacataagactaaagaatgtaaatactggagaagttagagaagttagagttttcaacttcaatactgaaaagatttatcactga
ggtgaattagaaattgattgtgttgttgaatgttcaggaagattcttaactaaggaagcagttaagtgtcaccttgatgcaggagctcaaaa
agttcttatttcagctcctgcaaaggatgacactaagacagttgtttacaacgtaaaccatactcaaattaccagctcagacaatgttatttc
aggagcttcatgtacaactaatgcactagctcctatcgtaaaaattattcacagaaaatttggaattaattctggattcatgacaacagttca
cgctttcacttctgaccaaagacttcaagactctcctcactctgacctaagaagagctagagcagctgctggatcaattattcctacaacta
caggagcagctgctgcaattggaagagtaattccagaattgaatggaaaacttgatggaattgcacacagagtgcctgtattaactggtt
ctctagttgacctatgtttaaaaataaataagtctgtttctgcagaagaaatcaatgaagcaattaaggatggagaaaatgaaacccttgctt
atgtagaagatccaattgtatctgctgacattatcggagatacacatggttctgtttttgactcatctctaactaaagtattgccaactggaga
agttaagctgtatgcatgatatgataatgagtcttcttatgtaaatcaacttgcaagaactttgaaatactacatttctctttaa;
SEQ ID NO: 339:
ttcttcccagagtggatttccgttggttccgcctccttcgtccgtcgtaatatcaggccttctgttctgttcgctgtctgtctagggcaccctta
ctgcaagagaagtatttgaggtcatatcgtcccatgaagtcgaccacctgtttcctctcttcactgtcacgtacgacatcgcattcaaggga
agagatccagcagatctcgttcgtgtattcgagacaagagaggtccgcccccacaagacggctgaagaatgcaacattcttgtgctgc
ctcctctcatggcaaatgccagaagaagggtacgtgttgcatcataacaagagctgtatttcccgctggcaaatacaggtgaaatgtacc
tccagaaaagccacctagtatcgtgcggcaatgtgccacctcgcctcttgggagaaaaagaggaagagacgctgccgctgttttgcaa
atgaaaaggattcattttcgcagtacaccaggagttggattttgtagagcgtctctcttcaagcagcgtattgtcgagaaaaaga;
SEQ ID NO: 340:
ctgcagggaggaagacgaaagttgtttttttatttttttttctttttgtttttctgatttttgttttttttgactcgggcccagctgcgtctgtcg
ggatgagaccgcggagccgaagtgcgttttctttttttgacttttttttgttttttcacaggcaagctcgcctgtgcttggagccacagaagggac
agaagtcgaaggggactacagacgcgatgccgctcctccagccgtcttggaggagagatatcaggactgtagatgaaggcgagggtg
aggatgagggggtggcgtggttgggaagcgacgagagtcggagagggagaagatgtttccggcttggctgcttttcctggagggtg
gaaaaagagacaccggaatgcgatccagacgagacgacgctttcctcgtggtgatggcggagagaattgaagagtggagaagagg
gcgagggagacagagtcggaggcttggacgaagggaggaggaggggtaggagaggaatccagatgcactgtgtctgcag;
SEQ ID NO: 341:
ttgctcaaaaaatttaaaaattttattctattttcatctatattttcaccaatagcatttgctatatcatgttctaatacaggagtagtcaagcaa
gaggatgtatcagttagtcaaggtcaatgagataaaagtataacatttggtgtttcagaagcttggttaaacaagaaaaaaggagataaaga
agttaacaaagaagttattaatacatttttagaaaatttcaaaaaagaatttaataaactcaaaaatgcaaatgataaaaccaaaaacttcga
tgacgttgattttaaagtaactccaattcaagactctactgtgttgttaaacaatttatctactgacaatcctgaattagattttggaattaatgct
tcaggaaaattggtagaatttctaaaaaataatcctggtataataactcctgcattagaaacaacaactaattcttttgtatttgacaaagaaa
aagataaattttatgttgatggtacagattcagatccacttgtaaaaattgctaaagaaattaataaaatttttgttgaaactccatatgcaagtt
gaactgatgaaaatcataagtgaaatggtaatgtttatcaaagtgtttacgatccaactgttcaagctaatttttatagaggaatgatttgaat
aaaaggtaatgatgaaactctagctaaaattaaaaaagcttgaaatgataaagattgaaatacatttagaaattttggaattttacacggtaa
agataattcttcttctaaattcaagttagaagaaactatattaaaaaaccactttcaaaataaatttacaacactaaatgaagacagaagcgc
acatccaaacgcatataaacaaaaatctgcagatacattgggaactttagatgatttccatattgctttttcagaagaaggttcttttgcttga
acacataacaaatcagcaacaaaaccttttgaaactaaagcaaatgaaaagatggaagcacttatagtaactaatccaattccgtatgatg
ttggagtgtttagaaaaagtgttaaccaattagaacaaaatttaattgttcaaacattcattaatttagctaaaaataaacaagatacatatggt
ccacttttagggtataatggttataaaaaaattgacaatttccaaaaagagattgtagaagtttatgaaaaagccattaaataa;
SEQ ID NO: 342:
gatgaacgctcgctgtgtgcctaatacatgcatgttgaacgggatgtagcaatacattcagtagcgaatgggtgagtaacacgtacctaa
cctacctttaagactgggataactattggaaacaatagctaataccggatatagttatttatcgcatgatgagtaatagaaaggagcttcac
agcttcacttaaaaatgggggtgcggaacattagttagttggtagggtaatggcctaccaagacgatgatgtttagccgggccgagagg
ctgtacggccacactgggactgagatacggcccagactcctacgggaggcagcagtaaggaattttccacaatgagcgaaagcttga
tggagcgacacagcgtgcaggatgaagttcttcggaatgtaaactgctgttataagggaagaaaaaatagaataggaaatgattttatctt
gacggtaccttattagaaagcgacggcaaactatgtgccagcagccgcggtaatacataggtcgcgagcgttatccggaattattggg
cgtaaagcgtccgtaggttttttgctaagtctggagttaaatgctgaagctcaacttcagtccgctttggatactggcaaaatagaattataa
agaggttagcggaattcctagtgaagcggtggaatgcgtagatattaggaagaacaccaataggcgaaggcagctaactggttatatat
tgacactaagggacgaaagcgtggggagcaaacaggattagataccctggtagtccacgccgtaaacgatgatcattagttggtggaa
taatttcactaacgcagctaacgcgttaaatgatccgcctgagtagtatgctcgcaagagtgaaacttaaaggaattgacgggaacccg
cacaagcggtggagcatgtggtttaatttgaagatacgcgtagaaccttacccactcttgacatcttctgcaaagctatagagatatagtg
gaggttaacagaatgacagatggtgcatggttgtcgtcagctcgtgtcgtgagatgttaggttaagtcctgcaacgagcgcaacccttttc
tttagttactaatattaagttaaggactctagagatactgcctgggtaaccaggaggaaggtggggacgacgtcaaatcatcatgcctctt
acgagtggggcaacacacgtgctacaatggtcggtacaaagagaagcaatatggtgacatggagcaaatctcaaaaaaccgatctca
gttcggattgaagtctgcaactcgacttcatgaagtcggaatcgctagtaatcgtagatcagctacgctacggtgaatacgttctcgggttt
t;
SEQ ID NO: 343:
atgaaacctattaaaatagctctaattggtgctggaaatgtcggaaattctttcctttatgcagcaatgaatcaaggacttgcatccgagtat
ggaattattgatattaaccctgattttgccgatggtaatgcttttgattttgaagatgcctcagcttctttgccttttccgattagtgtctcccgt
tatgaatataaagatctaaaagatgctgattttattgtaattacagcgggaagaccacaaaaaccgggtgaaactcggcttgaattagtagct
gataacatccgaattatccgggaaattgcactaaaagtcaaagaaagtggctttagtggaataagtattattgttgctaatcctgttgatata
attacaagggcttaccgggatgcttctggattttccgatcaaaaagttatcggtagtggaactgttttagatacagcaaggcttcaatttgca
atcgcaaaaagagcaaaagtatcgcctaattcggttcaggcctacgtgatgggtgaacatggtgattcatcttttgttgcttattcaaatatt
aaaattgccggtgaatgtttctgtgcttattctaaactaaccggaattgatagctcaaattacgaaaaagaacttgaatatccagtttctcgc
cgggcttatgaaattattaatcgtaaaagggcaacattttatggaattggtgcagctattgccaaaatagtttctaatattatcaaagatacaa
aaaatattatgattgccggagcaaatttacgaggagaatacggatttcacggagtaaatatcggagttccagttgttttaggggcaaacg
gaattgaaaaaattattgagattagttttaatgataaagaaaaagaaaaatttgccaaatcagttgcaatcattgataaaatttatcaggatgc
aattaaaaatatttaa;
SEQ ID.NO. 344:
ttactttttaacatagtttctaatcaacccgttaattaattcacttctttttgttaaacttcctttctctgcaaatgatttatcattaa
atattgccagcgtttttaatataattgtaggttcttttttagcgttttgttgtggttttttattttgttcgcctgggccgatttgataccaatcaa
gatgaaatcattctaaagtaaagaaatttggcaaggtgtctttagggttaatctcgaatttaagacgttttacacgttttgggtactttgttggaa
aaaaataagaatttaccttaatcgaatccttgccagcaagaatttcatataaataggtaccattctcaacaatttctttggttttaggtacattat
tatttgaattttgaactttaattacaagcccttttccttttctatcggctaaaagattaatccaattaccatcggaaatattttttgcatcaaaaa
ctaaataaattgctccggtatataaagaagttgatatttctttttctggattagacgattttgactgtaatttcaaaggtttctttagctctaaac
cttgatcagtttgaattgcatcttctgatttaaatattaatttattatcacttaaatcttttaggataaattcttttttatcttttccttgatttt
gataattaattgttccatctaaaaacacatcgggattgaatttcttaattatatcataggcagatttattagaattaataatattattaagactaa
ttttggactgagccaaaattagattttgattagttttatcaactaatttaaaatcaaaaaatagcttatttcctaaagttgagatctcatattcgc
tttttgtattttcaagattataaatattagcgaaattaaaagctgttcaaatgcttgtcccaaatttttcattttctaatacagaatttgagctgc
ttgtggaattaggaagtgaattttgactagtttgaggcgatgaagtggaaactggttgtgtttgctgactttgactttgacccttagcttcttctg
caaaagtgcttaccttgactgtttttgctgaattttctgttgttttagctagtgaaacctctggttttttcggttgtgtggggctatttttttctg
ttttttgagtatttgcctttgattttaagccctcaatcacggtttttagctcttgttttactttttcctggtcttggtcgactaaatattttcccg
tatttaataaataaatgcttttaagaacttcttcacttggattttccaaattagtcactgtttttgctagttcaaaaagtcgatcaactcgctgtt
ttatttcatttgaggataaagtttttgcatcttgatcttcaggcattgaaaattctttaactgcttttatcgttccaaacatttgggcaacagcgg
cttggtatttattaaaatcaacttttagatcaacaaaaactggaagatcctggatgataaattttttatcaatagcttttcgttttaaagaagcag
taatttcaaagcgaattttttgattttcaactagttttttcttatcaacaacagaaatctgacttggatctaaaagtttgggaataatacttaaat
agacagttccgattttggtattaagaaaaaaatttgtctcacttaaatcaaaaagatctttaaattctttactttttgttaaaatttgggatgtat
ttacttttgaattcggagatccatatcaataatcagagaggctaattttatcttttactagtgccatttgcaaatcatctttggtttttaatttgc
cttgaagatttgtttcaagtcaggattttagaattttagaaatttcctgatttgaaatcgcgccgcgaacttcaagatttaaattcatagtttttt
tagttgaaatttgcactaaattaagtccaagaactaattttccatctttatttaacaaagttaataatttttttcggtccaaaactgggtctaatt
gataatctgggcctaaaaatacgggttctcctaaggaattataaagtgaaagactcgcttttgatgcaaccaaagctctttcaagtcgtgaaaat
caattacttcctctttcattttcaaaattttgggcaagcaaaatttggaattccatcaaagttagatttgctcttgaaagatcaacaaaagacttg
cttgaatcaatttgactaaaatcaaaattagttctagcttgttctgtatttccaaagcctttaagtacaatttgttttgaataagttatttgatcc
ttatcagattttgcataaataacaacatttttaattgaattttgatcaactactgcattttcaagatcaaaacttactaaataatcattgtttat
tagttcacttaaatcaaaagcagaaacaagattataacgataaagatcacttttggcagcagttaaaactgtttttgccgatcattttttaaaatt
atctttaattttaagatttaaaacaattgaatcaaatttttcctgactaattttagcattttttgctacttttagctgactcgggatctgatttag
ataagaataatatgatttattataagaattaattccgacaactgtaccaacagcggctgataaactaagactaattccggcaattgttgttattaa
tcaaaaaggttttttaagtaattttgctaacttcat

Preferably, the primer group comprises a primer group 1 and a primer group 2, wherein the primer group 1 comprises primer sequences set forth in singular sequence numbers in SEQ ID NO: 1 to SEQ ID NO: 302, and the primer group 2 comprises primer sequences set forth in double sequence numbers in SEQ ID NO: 1 to SEQ ID NO: 302.

The present invention further provides a multiplex PCR reagent for simultaneous detection of 15 porcine pathogens through high-throughput targeted amplicon sequencing, comprising a PCR reagent 1 and a PCR reagent 2, wherein the PCR reagent 1 comprises the primer group 1, and the PCR reagent 2 comprises the primer group 2.

The present invention mainly aims at a specific primer of the pathogenic gene. However, because the sequencing platform requirements of each sequencing company are inconsistent, other corresponding reagents can be added according to different platforms when the primer sequence is synthesized. The PCR reagent 1 and the PCR reagent 2 further comprise necessary reagents required in the PCR amplification process, and the requirements on an amplification system are different and can be adjusted adaptively.

Preferably, each primer in the primer group 1 has a concentration of 10 nmol/μL in the PCR reagent 1, and each primer in the primer group 2 has a concentration of 10 nmol/μL in the PCR reagent 2.

The present invention further provides a kit for simultaneous detection of 15 porcine pathogens through high-throughput targeted amplicon sequencing, comprising the primer group or the multiplex PCR reagent.

The present invention further provides use of the primer group or the multiplex PCR reagent or the kit in constructing a high-throughput amplicon sequencing library for simultaneous detection of 15 porcine respiratory pathogens.

The present invention further provides use of the primer group or the multiplex PCR reagent or the kit in high-throughput amplicon sequencing for simultaneous detection of 15 porcine respiratory pathogens.

The present invention further provides use of the primer group or the multiplex PCR reagent or the kit in detecting 15 porcine respiratory pathogenic genes in a test sample.

The present invention further provides a method for detecting 15 porcine respiratory pathogenic genes in a test sample, which aims at non-disease diagnosis and treatment and comprises the following steps:
1) performing multiplex PCR reagent on a test sample by using the PCR reagent 1 and the PCR reagent 2 in the multiplex PCR reagent separately to obtain a multiplex PCR amplification product 1 and a multiplex PCR amplification product 2;
2) mixing the multiplex PCR amplification product 1 and the multiplex PCR amplification product 2 to prepare an amplicon library; and
3) sequencing the amplicon library to detect 15 porcine respiratory pathogenic genes in the test sample.

The present invention has the technical effects and advantages as follows.

The present invention provides a primer group for simultaneous detection of 15 porcine pathogens through high-throughput targeted amplicon sequencing and use thereof, wherein the pathogens comprise 7 viral pathogens, namely, African swine fever virus, porcine reproductive and respiratory syndrome virus (North American type NA/European type EU), pseudorabies virus, porcine circovirus (type 2 and type 3), swine fever virus, foot and mouth diseases virus, and swine influenza A virus; 4 bacterial pathogens, namely, haemophilus parasuis, Pasteurella multocida, Actinobacillus pleuropneumoniae, and Streptococcus suis; and 4 other types of pathogens, namely, Eperythrozoon suis, Toxoplasma gondii, Mycoplasma hyorhinis, and Mycoplasma hyopneumoniae. The present invention particularly relates to target genes corresponding to target pathogens, wherein each pathogen comprises 2 to 8 different target genes, and the total number of the target genes is 42, so that the present invention aims to implement the simultaneous and rapid detection of 15 pathogens by sequencing the target genes of the target pathogens, further implements the simultaneous and rapid detection of multiple pathogens of diseased pigs, and closely associate diseased animals, infection pathogens, and the pathogenic target genes.

The present invention comprises a primer combination that is called Primer Pool and comprises 151 pairs of primers in total, a size range of amplicon fragments corresponding to each pair of primers is 100-150 bp, which can efficiently and specifically amplify pathogenic target genes; in addition, the present invention further provides an amplicon sequencing detection analysis method for 42 target genes of 15 porcine respiratory pathogens, which has the technical advantages of rapidness, high efficiency, high targeting, strong specificity, and the like, and is suitable for rapid and accurate simultaneous diagnosis of multiple pathogens of diseased pigs in pig farms.

### BRIEF DESCRIPTION OF THE DRAWINGS

FIG. 1 is a simplified flow chart of high-throughput amplicon sequencing;

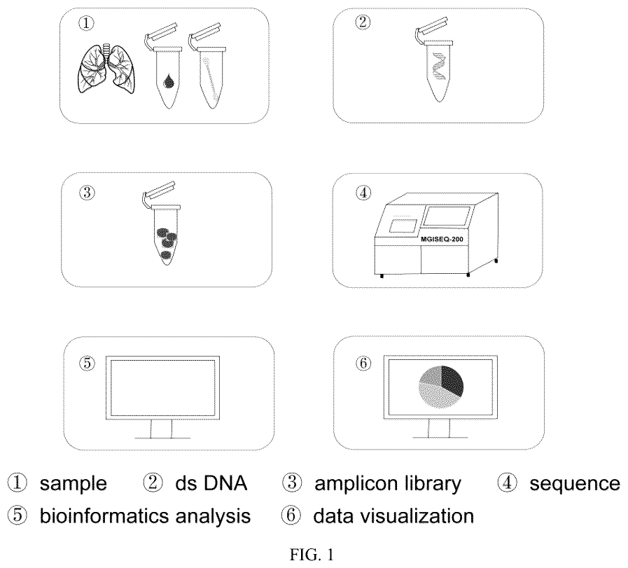

FIG. 2 is a schematic diagram of amplicon sequencing library construction;

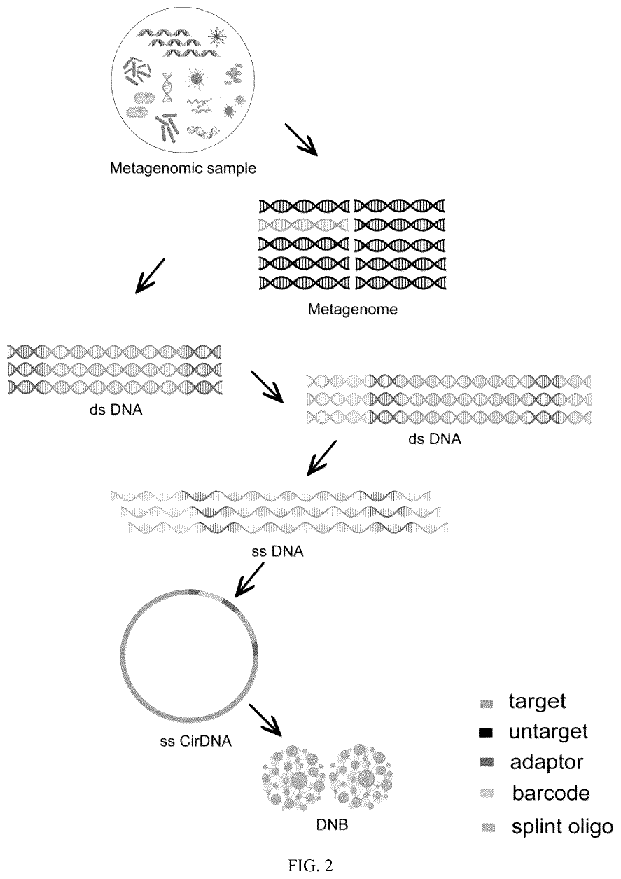

FIG. 3 shows details of pathogens and target genes thereof contained in the amplicon sequencing panel;

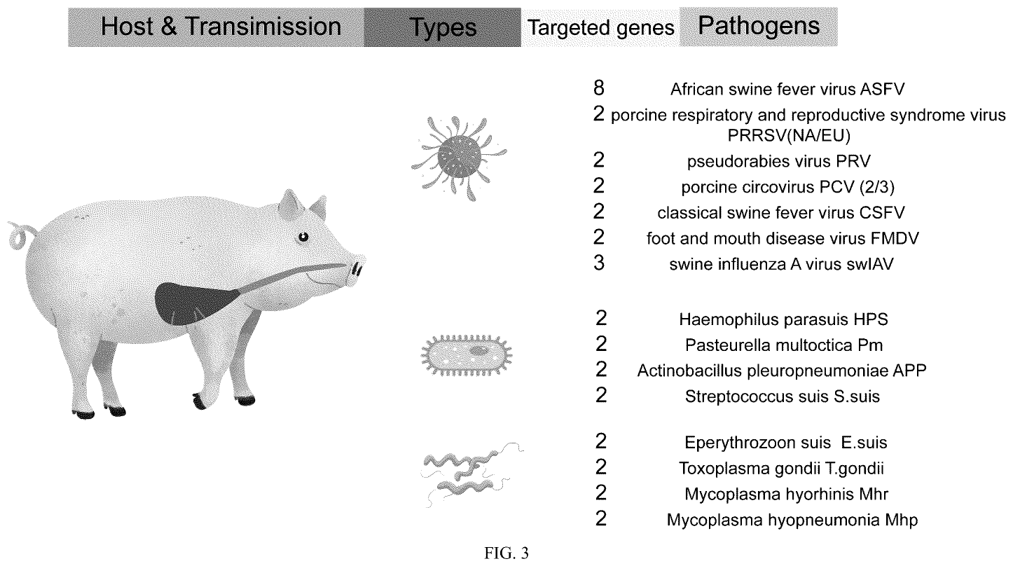

FIG. 4 shows distribution of target genes detected in Embodiment 1; and

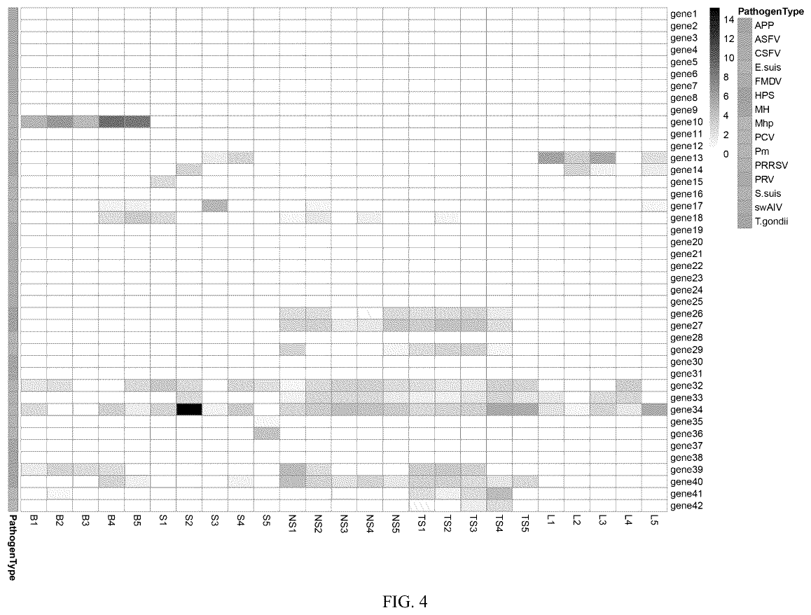

FIG. 5 shows detection results of pathogens in Embodiment 1.

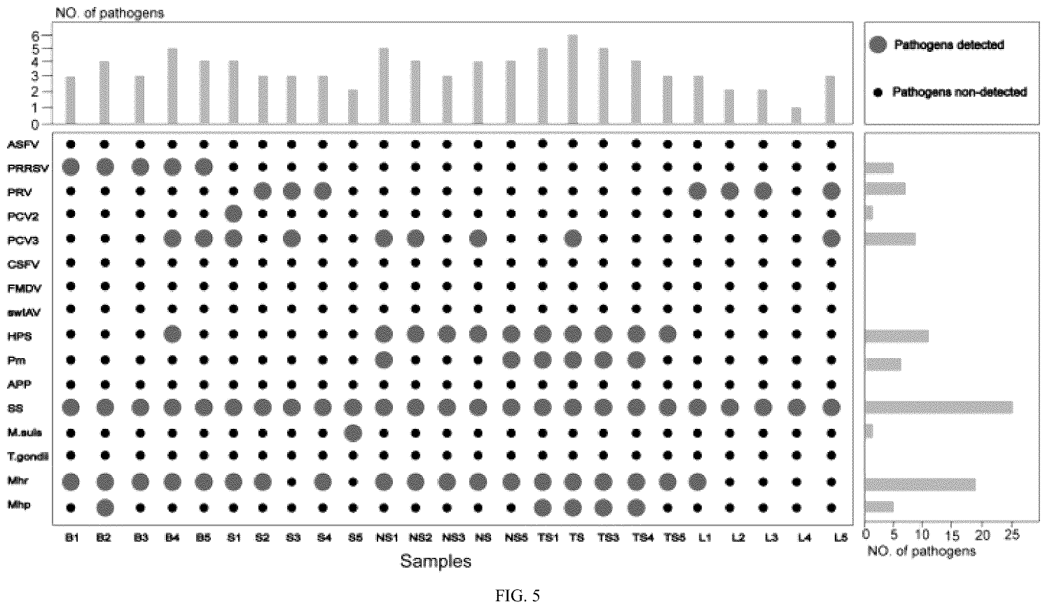

### DETAILED DESCRIPTION OF THE EMBODIMENTS

Unless otherwise stated, the methods used in the following examples are conventional methods.

The materials, reagents, and the like used in the following examples can be commercially available unless otherwise stated.

The technical solutions provided by the present invention will be described in detail below with reference to the examples, which, however, should not be construed as limiting the scope of the present invention.

TABLE 1
Main reagents and instruments
Main reagents

| Name | Supplier | Cat. No. |
|---|---|---|
| HiPure MicroBiome DNA Kit | Magen | D3148-03 |
| TIANamp Virus DNA/RNA Kit | TIANGEN | DP315 |
| GoScript ™ Reverse | Promega | A2801 |
| Transcription Mix, Random | | |
| Primers | | |
| ATOPlex customized Panel | MGI | Customization |
| ATOPlex DNA multiplex PCR | MGI | 1000021191 |
| universal library constructing | | |
| module | | |
| ATOPlex single-label primer | MGI | 1000024934 |
| module (01-96) | | |
| MGIEasy circularization module | MGI | 1000005260 |
| MGIEasy DNA purification | MGI | 1000007325 |
| magnetic bead kit | | |
| MGISEQ-200RS high-throughput | MGI | 1000019846 |
| sequencing reagent set | | |
| (FCS PE100) | | |

Instruments

| NAMES | BRANDS |
|---|---|
| MGISEQ-200 | MGI |
| PCR instrument | ABI |
| Qubit ™ 4 Fluorometer | Thermo Fisher |
| NanoDrop | Thermo Fisher |
| Minute Centrifuge (MINI-6KC) | MIULAB |
| Vortex Mixers | MIULAB |
| DynaMag ™-2 | Invitrogen by Fisher Scientific |
| Pipettor | Eppendorf |

### Embodiment 1

1. Preparation of Primer Group for Simultaneous Detection of 15 Porcine Pathogens Through High-Throughput Targeted Amplicon Sequencing

According to literature reference, common porcine pathogens were selected through statistics and analysis, pathogenic target genes were determined, a complete gene sequence was downloaded on NCBI (https://www.ncbi.nlm.nih.gov/) according to the names of the target genes, a specificity of the target genes was further determined through NCBI Blast, and finally the sequences of the pathogenic target genes were obtained, wherein the nucleotide sequences of the pathogenic target genes were set forth in SEQ ID NO: 303 to SEQ ID NO: 344.

Finally, designing, analysis and comparison were performed many times according to the target gene sequences, wherein each gene was designed with 2-4 pairs of specific primers, and the total number of primers was 151 pairs of primers, and the nucleotide sequences of the primers were set forth in SEQ ID NO: 1 to SEQ ID NO: 302. The primers were synthesized by MGI Tech Co., Ltd., according to the company's library construction kit combined with this group of specific primers, the specific adapter sequence “GACATGGCTACGATCCGACTT” (SEQ ID NO: 345) was added to a 5′-end of each forward primer (F primer), the specific adapter sequence “CGCTTGGCCTCCGACTT” (SEQ ID NO: 346) was added to a 5′-end of each reverse primer (R primer), and finally the primer pool sequence for constructing the amplicon sequencing library was obtained.

TABLE 2
Sequences of a primer group for simultaneous detection of 15 porcine pathogens
through high-throughput targeted amplicon sequencing

| | F ID | F seq |
|---|---|---|
| SEQ ID NO: 1 | ASFV-p72-F-1 | GACTCAAAGTGGGTTCGGG |
| SEQ ID NO: 3 | ASFV-p72-F-2 | AAAATGACTGGATATAAGCACTTGGTTG |
| SEQ ID NO: 5 | ASFV-p72-F-3 | AGCGAACGCGTTTTACAAAA |
| SEQ ID NO: 7 | ASFV-p72-F-4 | CGCAGCACAGCTGAACC |
| SEQ ID NO: 9 | ASFV-p54-F-1 | AAACTGTATATCTTCCTCCTCAATAGCA |
| SEQ ID NO: 11 | ASFV-p54-F-2 | CGTAACTGGGTTGTCCGTAA |
| SEQ ID NO: 13 | ASFV-p54-F-3 | GCAGACCGGCAACAAACAG |
| SEQ ID NO: 15 | ASFV-p54-F-4 | ATGCGTATAGGTGTTTCTTTGTCG |
| SEQ ID NO: 17 | ASFV-EP402R-F-1 | TGACATTGTTCTTCTTCATTAGATTCAG |
| SEQ ID NO: 19 | ASFV-EP402R-F-2 | GGACATGGTTTGGGTGGAG |
| SEQ ID NO: 21 | ASFV-EP402R-F-3 | TACATGCGTCCCTCAACACA |
| SEQ ID NO: 23 | ASFV-EP402R-F-4 | GTGAATAAGCGAAATATTTTGGGTAGA |
| SEQ ID NO: 25 | ASFV-MGF505-1R-F-1 | ACAGTTGTTTCCTGCTCGTTGAATA |
| SEQ ID NO: 27 | ASFV-MGF505-1R-F-2 | TGTTGCCACTTACAATTCAACAA |
| SEQ ID NO: 29 | ASFV-MGF505-1R-F-3 | AGGTAAAAACGCTCATGATGCTA |
| SEQ ID NO: 31 | ASFV-MGF505-1R-F-4 | TTATGGGCAATAGCACATTCAAAGGTA |
| SEQ ID NO: 33 | ASFV-MGF505-2R-F-1 | TGAGATCCCGTCGTATGAGT |
| SEQ ID NO: 35 | ASFV-MGF505-2R-F-2 | GCAGAAAAATATCTTACCATCTTTTCCA |
| SEQ ID NO: 37 | ASFV-MGF505-2R-F-3 | TTGCTCAAAACGCGGCCA |
| SEQ ID NO: 39 | ASFV-MGF505-2R-F-4 | CTTTAGAACGTTTTTGTGGTGTTTCATG |
| SEQ ID NO: 41 | ASFV-MGF360-13L-F-1 | CCGCTCTCTCTGCAGACC |
| SEQ ID NO: 43 | ASFV-MGF360-13L-F-2 | TTTGGATATTTACAGCTCAGATGCTTC |
| SEQ ID NO: 45 | ASFV-MGF360-13L-F-3 | CCAAAGAAACTCTAGAGGACAACG |
| SEQ ID NO: 47 | ASFV-MGF360-13L-F-4 | GACATATCATGTCATGGACGTCT |
| SEQ ID NO: 49 | ASFV-MGF360-14L-F-1 | AGTGACTATGATTATACGTTGCAGC |
| SEQ ID NO: 51 | ASFV-MGF360-14L-F-2 | GTTTCCAAGAGTGGATTATGACAAAA |
| SEQ ID NO: 53 | ASFV-MGF360-14L-F-3 | CCCTGGCATTACGACATAATTTTACAAA |
| SEQ ID NO: 55 | ASFV-K205R-F-1 | TTGAGCCACGCGAACAGT |
| SEQ ID NO: 57 | ASFV-K205R-F-2 | AAAAACAACGATTAAGGAGGCTATGC |
| SEQ ID NO: 59 | ASFV-K205R-F-3 | CGAGAAAAGTTTTTAACACCAGAAATTC |
| SEQ ID NO: 61 | PRRSV(NA)-orf6-F-1 | CGTCTCTAGACGACTTCTGC |
| SEQ ID NO: 63 | PRRSV(NA)-orf6-F-2 | TGAAAGCCCGCGGCAC |
| SEQ ID NO: 65 | PRRSV(NA)-orf6-F-3 | CAAGGTTTACCACTCCCTGCT |
| SEQ ID NO: 67 | PRRSV(NA)-orf7-F-1 | AACAACGGCAGACAGCAAA |
| SEQ ID NO: 69 | PRRSV(NA)-orf7-F-2 | CCATTTCCCTCTAGCGACTGAAG |
| SEQ ID NO: 71 | PRRSV(EU)-orf6-F-1 | AGTCGGCCGCGTGACA |
| SEQ ID NO: 73 | PRRSV(EU)-orf6-F-2 | CTGTGGGGTATTTATAGCTTCACAGA |
| SEQ ID NO: 75 | PRRSV(EU)-orf6-F-3 | GGTAACCGAGCATACGCTGTG |
| SEQ ID NO: 77 | PRRSV(EU)-orf7-F-1 | TCAGGCTTTTTCTTTTTGGCCTG |
| SEQ ID NO: 79 | PRRSV(EU)-orf7-F-2 | ACCTTCCCGCTGGATGAA |
| SEQ ID NO: 81 | PRV-gE-F-1 | CCGTCCACGTAGATCGGG |
| SEQ ID NO: 83 | PRV-gE-F-2 | GAGGGCCGAGCCGAAG |
| SEQ ID NO: 85 | PRV-gE-F-3 | CAGACGTCGAAGCCGGAG |
| SEQ ID NO: 87 | PRV-gE-F-4 | GGCGAGAAGAGCTGCG |
| SEQ ID NO: 89 | PRV-gB-F-1 | TGGCGGTCTTTGGCGC |
| SEQ ID NO: 91 | PRV-gB-F-2 | ACCTCCTCGACGATGCAG |
| SEQ ID NO: 93 | PRV-gB-F-3 | CTGCAGTTCACCTACGACCAC |
| SEQ ID NO: 95 | PRV-gB-F-4 | CCGCAACCCCATGAAGG |
| SEQ ID NO: 97 | PCV2-cap-F-1 | TCCAAGGAGGCGTTACCGAA |
| SEQ ID NO: 99 | PCV2-cap-F-2 | TCAATAGTGGAATCTAGGACAGGT |
| SEQ ID NO: 101 | PCV2-cap-F-3 | TCCTACCACTCCCGGTACTTTAC |
| SEQ ID NO: 103 | PCV2-rep-F-1 | GCAAGAAGAGTGGAAGAAGCG |
| SEQ ID NO: 105 | PCV2-rep-F-2 | GCAGTATTCTGATTACCAGCAATCA |
| SEQ ID NO: 107 | PCV2-rep-F-3 | CTTACTGATAGAATGTGGAGCTCCTAGA |
| SEQ ID NO: 109 | PCV2-rep-F-4 | GCCAGCCATAAAAGTCATCAATAAC |
| SEQ ID NO: 111 | PCV3-cap-F-1 | TGGTGGAGTATTTCTTTGTGTAGTATGT |
| SEQ ID NO: 113 | PCV3-cap-F-2 | GATACTAAAGATGAAAGTTACACTCAGC |
| SEQ ID NO: 115 | PCV3-cap-F-3 | CCGTAGAAGTCTGTCATTCCAGTTTT |
| SEQ ID NO: 117 | PCV3-rep-F-1 | GAGGGAAAGCCCGAAACAC |
| SEQ ID NO: 119 | PCV3-rep-F-2 | GGAGTGGTATTCATCGGAGAATATTCG |
| SEQ ID NO: 121 | PCV3-rep-F-3 | CCCGACGTCTCCGTCAG |
| SEQ ID NO: 123 | PCV3-rep-F-4 | GTAAACCGCGTGATTTTAAAACTG |
| SEQ ID NO: 125 | CSFV-5′UTR-F-1 | GCAAGTCACAGCGCACT |
| SEQ ID NO: 127 | CSFV-5′UTR-F-2 | TTGATGATATTGAGTTTTGCTCCCATAC |
| SEQ ID NO: 129 | CSFV-5′UTR-F-3 | GGACCAACTTCATCTCAAGACTTGTA |
| SEQ ID NO: 131 | CSFV-5′UTR-F-4 | TGGATTTTGTGACATGATCTCCAT |
| SEQ ID NO: 133 | CSFV-E2-F-1 | AGTGCTTGATCGGTAACACAACT |
| SEQ ID NO: 135 | CSFV-E2-F-2 | GTTCTGTTAGAACTATGTAGGTCACTAT |
| SEQ ID NO: 137 | CSFV-E2-F-3 | GAAGTGAGTGAAGCCTCCTTAT |
| SEQ ID NO: 139 | CSFV-E2-F-4 | TGTGGTGGTCGCACAATC |
| SEQ ID NO: 141 | FMDV-polyprotein-F-1 | CCCAGTGGCCAATTCCTCA |
| SEQ ID NO: 143 | FMDV-polyprotein-F-2 | TTCAAAGCGACAGGCTTCTT |
| SEQ ID NO: 145 | FMDV-polyprotein-F-3 | ACACTTCCACATGGATTATGGAACTG |
| SEQ ID NO: 147 | FMDV-polyprotein-F-4 | GGTTGTGTCTGTGGATCCC |
| SEQ ID NO: 149 | FMDV-5′UTR-F-1 | GAAAGGCGCCGGCCTC |
| SEQ ID NO: 151 | FMDV-5′UTR-F-2 | GGAACCGATGGACTTTCGTTCA |
| SEQ ID NO: 153 | FMDV-5′UTR-F-3 | TTCTTGTCCGAAGTTAGAGGGCT |
| SEQ ID NO: 155 | FMDV-5′UTR-F-4 | ACCATGCTCCGCTACGA |
| SEQ ID NO: 157 | swIAV-M-F-1 | GGATTGGTCTTGTCTTTAGCCA |
| SEQ ID NO: 159 | swIAV-M-F-2 | GACTGCAGCGTAGACGCTTT |
| SEQ ID NO: 161 | swIAV-M-F-3 | GATCCAGCCATTTGTTCCATG |
| SEQ ID NO: 163 | swIAV-M-F-4 | CATTCGTTTCTGATAGGCCTGTA |
| SEQ ID NO: 165 | swIAV-H1-F-1 | ACTGTTACATTCTTTTCTAGTACTGTGT |
| SEQ ID NO: 167 | swIAV-H1-F-2 | ATACCATCCATCTATCATTCCTGTCC |
| SEQ ID NO: 169 | swIAV-H1-F-3 | TGGTAAATCCTTGTTGATTCCAGCTTTA |
| SEQ ID NO: 171 | swIAV-H1-F-4 | TCTGGTAGAGACTTTGTTGGTCAGT |
| SEQ ID NO: 173 | swIAV-H3-F-1 | AAACTGTAATCCCGAATATCGGATCTAG |
| SEQ ID NO: 175 | swIAV-H3-F-2 | GTACGGTTTCAGGCATCAAAATTCTG |
| SEQ ID NO: 177 | swIAV-H3-F-3 | ATTCCTTATTTTGGAAGCCATCAC |
| SEQ ID NO: 179 | HPS-vtaA-F-1 | TTCTACGATAAATTTCAATGTTATCGCC |
| SEQ ID NO: 181 | HPS-vtaA-F-2 | AGAACAATCTATTGTCTTAGGCACAGG |
| SEQ ID NO: 183 | HPS-vtaA-F-3 | CTTGTCCTGGATCGCCTTTATC |
| SEQ ID NO: 185 | HPS-vtaA-F-4 | ATACCCGCTCTTGCATTAACAATATC |
| SEQ ID NO: 187 | HPS-infB-F-1 | AGTAGCAGCAGGTGAAGCT |
| SEQ ID NO: 189 | HPS-infB-F-2 | ACGGATATCGTTGTTCTTGTAGTAGC |
| SEQ ID NO: 191 | HPS-infB-F-3 | AACAAGAGTTATTACAACACGAAGTGAT |
| SEQ ID NO: 193 | Pm-plpE-F-1 | AAGAAGGCTCACTATTTGATTGAACC |
| SEQ ID NO: 195 | Pm-plpE-F-2 | TTGTCGGGTCCTGTAGGG |
| SEQ ID NO: 197 | Pm-plpE-F-3 | CCTTTAAAGCCCCTCTAGAACAAGAAA |
| SEQ ID NO: 199 | Pm-kmt1-F-1 | TTCGCTGCAATCGGTTCAT |
| SEQ ID NO: 201 | Pm-kmt1-F-2 | TGATAGCCATGCAATGCCA |
| SEQ ID NO: 203 | Pm-kmt1-F-3 | TTTGATGTGGTAGGACTGCCA |
| SEQ ID NO: 205 | Pm-kmt1-F-4 | AGTTTCATTTTCGCCTAACCCA |
| SEQ ID NO: 207 | APP-om1A-F-1 | GGACATATCAACCTTAGGTGTAAGTTCC |
| SEQ ID NO: 209 | APP-om1A-F-2 | GGTATTTTCGGTAAAAATGGCGAAGTA |
| SEQ ID NO: 211 | APP-om1A-F-3 | AAAAATATGGCTCCACAAATGGGTAATC |
| SEQ ID NO: 213 | APP-om1A-F-4 | CTTCTACAGAAGCCTTTAATTTCTGATT |
| SEQ ID NO: 215 | APP-apxIVA-F-1 | AATAAACCGGACGGAAATAATGGGAATA |
| SEQ ID NO: 217 | APP-apxIVA-F-2 | CGATAAGTTATTATCTTTTTGGCTACCT |
| SEQ ID NO: 219 | APP-apxIVA-F-3 | GGATTTACTCGAACGCGCCTATA |
| SEQ ID NO: 221 | APP-apxIVA-F-4 | TGCTCATTCGATAAACGAATATTTTCT |
| SEQ ID NO: 223 | S.suis-gdh-F-1 | GCTTCAAAAACAGGCTCAAGTGTA |
| SEQ ID NO: 225 | S.suis-gdh-F-2 | GTATCGACTTCGACCTCTTGGTG |
| SEQ ID NO: 227 | S.suis-gdh-F-3 | GTTAGCACCTGCAAGGTAGT |
| SEQ ID NO: 229 | S.suis-gdh-F-4 | GATCGGTTACATGTACGGTCAATACAA |
| SEQ ID NO: 231 | S.suis-recN-F-1 | CCTGCTCGTTCAACCAATTCTTTC |
| SEQ ID NO: 233 | S.suis-recN-F-2 | AAAGGAAAAGAGCCCTTCAATCTCA |
| SEQ ID NO: 235 | S.suis-recN-F-3 | GAGACGGACACGAGAAACG |
| SEQ ID NO: 237 | S.suis-recN-F-4 | GAGCAGTTTATCGCGTTCCTGA |
| SEQ ID NO: 239 | S.suis-gapdh-F-1 | TATTTCAACAAGTGTGCAAGCATTACTG |
| SEQ ID NO: 241 | S.suis-gapdh-F-2 | GAAGTACTCAAGAGTACGAACAAGTTG |
| SEQ ID NO: 243 | S.suis-gapdh-F-3 | CCTGAGATAACTGTTTCAGTACCAT |
| SEQ ID NO: 245 | S.suis-gapdh-F-4 | ACAGAACCTGTTGGAACTGGA |
| SEQ ID NO: 247 | E.suis-ppa-F-1 | TACAGAGAAATTAGATAGCACTACTACA |
| SEQ ID NO: 249 | E.suis-ppa-F-2 | ATGGATATATTTCTGATACTCTAGCAGA |
| SEQ ID NO: 251 | E.suis-ppa-F-3 | AAAAGTTTCTCAATTCAGCAATTCAAT |
| SEQ ID NO: 253 | E.suis-g1-F-1 | TGAGTAAGAACTTTAATGTCAACTACGT |
| SEQ ID NO: 255 | E.suis-g1-F-2 | AATGAAACCCTTGCTTATGTAGAAGATC |
| SEQ ID NO: 257 | E.suis-g1-F-3 | GGTAATTTGAGTATGGTTTACGTTGTA |
| SEQ ID NO: 259 | E.suis-g1-F-4 | AATTAATTCTGGATTCATGACAACAGTT |
| SEQ ID NO: 261 | T.gondii-B1-F-1 | AGGAAACAGGTGGTCGACT |
| SEQ ID NO: 263 | T.gondii-B1-F-2 | GCATTTGCCATGAGAGGAGG |
| SEQ ID NO: 265 | T.gondii-B1-F-3 | GCAGCGTCTCTTCCTCTTTT |
| SEQ ID NO: 267 | T.gondii-B1-F-4 | CACCTAGTATCGTGCGGCAAT |
| SEQ ID NO: 269 | T.gondii-RE-F-1 | TCGGCTCCGCGGTCTC |
| SEQ ID NO: 271 | T.gondii-RE-F-2 | CTTCTCCCTCTCCGACTCTC |
| SEQ ID NO: 273 | T.gondii-RE-F-3 | TGAAGGCGAGGGTGAGGAT |
| SEQ ID NO: 275 | T.gondii-RE-F-4 | CCCTCCTCCTCCCTTCG |
| SEQ ID NO: 277 | Mhr-p37-F-1 | TTCAAAAGGTTTTGTTGCTGATTTGTTA |
| SEQ ID NO: 279 | Mhr-p37-F-2 | AAAAGTGGACCATATGTATCTTGTTTAT |
| SEQ ID NO: 281 | Mhr-p37-F-3 | CATTTGGTGTTTCAGAAGCTTGGTTAA |
| SEQ ID NO: 283 | Mhr-16SrRNA-F-1 | GCTCGCTGTGTGCCTAATAC |
| SEQ ID NO: 285 | Mhr-16SrRNA-F-2 | GTCGTCCCCACCTTCCTC |
| SEQ ID NO: 287 | Mhr-16SrRNA-F-3 | CGCCTATTGGTGTTCTTCCTA |
| SEQ ID NO: 289 | Mhp-p36-F-1 | AGATCTTTATATTCATAACGGGAGACAC |
| SEQ ID NO: 291 | Mhp-p36-F-2 | GAATTATCCGGGAAATTGCACTAAAAGT |
| SEQ ID NO: 293 | Mhp-p36-F-3 | GGCGAGAAACTGGATATTCAAGTT |
| SEQ ID NO: 295 | Mhp-p102-F-1 | TGGACTGAGCCAAAATTAGATTTTG |
| SEQ ID NO: 297 | Mhp-p102-F-2 | ACTTTAGAATGATTTCATCTTGATTGGT |
| SEQ ID NO: 299 | Mhp-p102-F-3 | TTAGCCTCTCTGATTATTGATATGGATC |
| SEQ ID NO: 301 | Mhp-p102-F-4 | TTTGGCAGCAGTTAAAACTGTT |
| | R ID | R seq |
| SEQ ID NO: 2 | ASFV-p72-R-1 | GCATCAGGAGGAGCTTTTTGTCTTA |
| SEQ ID NO: 4 | ASFV-p72-R-2 | GCTGCGTATCATTTTCATCGG |
| SEQ ID NO: 6 | ASFV-p72-R-3 | GCTTTAAACCATGGTTTATCCCAGG |
| SEQ ID NO: 8 | ASFV-p72-R-4 | CCAGTGGTCATATTAACGTATCCAGAG |
| SEQ ID NO: 10 | ASFV-p54-R-1 | CCAGTCACTACACCAAGCTTCTT |
| SEQ ID NO: 12 | ASFV-p54-R-2 | ATCAGCAGTGGGTAGAAGTCACT |
| SEQ ID NO: 14 | ASFV-p54-R-3 | CGGATGAGCAGGAGCACT |
| SEQ ID NO: 16 | ASFV-p54-R-4 | ATGGCAACTGGCGGGC |
| SEQ ID NO: 18 | ASFV-EP402R-R-1 | ATATTCCATTAAGCATCATAATTGGGAT |
| SEQ ID NO: 20 | ASFV-EP402R-R-2 | CATACATGAACCATCTCCCAGAG |
| SEQ ID NO: 22 | ASFV-EP402R-R-3 | CTAGGTAGTGGTTTGGGTGGAGA |
| SEQ ID NO: 24 | ASFV-EP402R-R-4 | CCTAAACCATGTCCTTCAGCTGAAT |
| SEQ ID NO: 26 | ASFV-MGF505-1R-R-1 | GTTCTCTCTCCAGAACTTATGTCG |
| SEQ ID NO: 28 | ASFV-MGF505-1R-R-2 | GAAAATGTATCCATACATCGAACGACA |
| SEQ ID NO: 30 | ASFV-MGF505-1R-R-3 | TTAGAATAAATGGATGGATAGTCCTGTA |
| SEQ ID NO: 32 | ASFV-MGF505-1R-R-4 | GAGGATGATTTGCCCTTCACTCATT |
| SEQ ID NO: 34 | ASFV-MGF505-2R-R-1 | TTTCCCTTCAAGACCTTTGCCG |
| SEQ ID NO: 36 | ASFV-MGF505-2R-R-2 | AACTACTCCGTGAAACGCAT |
| SEQ ID NO: 38 | ASFV-MGF505-2R-R-3 | AGATTTCCAGGATACTATAGCAAGATTT |
| SEQ ID NO: 40 | ASFV-MGF505-2R-R-4 | CATAGGCATGCTAGTTCAAATAGGGT |
| SEQ ID NO: 42 | ASFV-MGF360-13L-R-1 | GATTTTATTTGTATTCGGCCACGAT |
| SEQ ID NO: 44 | ASFV-MGF360-13L-R-2 | AGAATATTCCCAAATGATGGATAAGTTT |
| SEQ ID NO: 46 | ASFV-MGF360-13L-R-3 | GCCCCTTAATTGTATAACAAATTTGTTT |
| SEQ ID NO: 48 | ASFV-MGF360-13L-R-4 | GTAAGGACGCCATTTGTTTTCT |
| SEQ ID NO: 50 | ASFV-MGF360-14L-R-1 | CTTTACTAGCGCCGCTTCGA |
| SEQ ID NO: 52 | ASFV-MGF360-14L-R-2 | CTTAACGCCTGTATCCGAACCC |
| SEQ ID NO: 54 | ASFV-MGF360-14L-R-3 | TGCAAATCTCTTTTTCACGACACAAC |
| SEQ ID NO: 56 | ASFV-K205R-R-1 | TATCGTAACGTTCTATAAAGTTTTCGAA |
| SEQ ID NO: 58 | ASFV-K205R-R-2 | CGCTACGCGTGAAGAACAT |
| SEQ ID NO: 60 | ASFV-K205R-R-3 | ATCATCTCTTTGACCATTTTCTTCTGTT |
| SEQ ID NO: 62 | PRRSV(NA)-orf6-R-1 | ATTCAGAAAGATCAAAAGGTGCAG |
| SEQ ID NO: 64 | PRRSV(NA)-orf6-R-2 | CTTCTTTGGGGAGTGTACTCAGC |
| SEQ ID NO: 66 | PRRSV(NA)-orf6-R-3 | TTGTCGTCCGGCGTCC |
| SEQ ID NO: 68 | PRRSV(NA)-orf7-R-1 | GGCTTCTCCGGGTTTCTATTCTT |
| SEQ ID NO: 70 | PRRSV(NA)-orf7-R-2 | TGTAACTTATTCTCCCTGAGTCTG |
| SEQ ID NO: 72 | PRRSV(EU)-orf6-R-1 | AAGCCTAGATGATTTCTGCTTCG |
| SEQ ID NO: 74 | PRRSV(EU)-orf6-R-2 | GGAGACCTGCAGCACTTTC |
| SEQ ID NO: 76 | PRRSV(EU)-orf6-R-3 | GGCCATACTTGACGAGGTTAA |
| SEQ ID NO: 78 | PRRSV(EU)-orf7-R-1 | CGGTAAGAACCAGAGCCAGAAG |
| SEQ ID NO: 80 | PRRSV(EU)-orf7-R-2 | CTGAAGATGACATCCGGCACC |
| SEQ ID NO: 82 | PRV-gE-R-1 | CCACCTGGGACTACACGCT |
| SEQ ID NO: 84 | PRV-gE-R-2 | TCTCCGCCGAGACGAC |
| SEQ ID NO: 86 | PRV-gE-R-3 | GGGTCGTACGCGAGCC |
| SEQ ID NO: 88 | PRV-gE-R-4 | CCTCTACACGCTGCACGAC |
| SEQ ID NO: 90 | PRV-gB-R-1 | CGAGCGCCAGCAGCAG |
| SEQ ID NO: 92 | PRV-gB-R-2 | CCTGAACGCGCTCGGC |
| SEQ ID NO: 94 | PRV-gB-R-3 | CAGGCGCGACATCTCG |
| SEQ ID NO: 96 | PRV-gB-R-4 | GATGGACATGTACCGGATCATGT |
| SEQ ID NO: 98 | PCV2-cap-R-1 | GGGAGAGGCGGGTGTTGA |
| SEQ ID NO: 100 | PCV2-cap-R-2 | TAGATGATAACTTTGTAACAAAGGCCAC |
| SEQ ID NO: 102 | PCV2-cap-R-3 | TGGTCTACATTTCCAGCAGTTT |
| SEQ ID NO: 104 | PCV2-rep-R-1 | CTCCTCATTGCCTTCCTCG |
| SEQ ID NO: 106 | PCV2-rep-R-2 | GTGGATTGTTCTGTAGCATTCTTC |
| SEQ ID NO: 108 | PCV2-rep-R-3 | GCGGAAATTTCTGACAAACGT |
| SEQ ID NO: 110 | PCV2-rep-R-4 | GAAAAAGCAAATGGGCTGCTAATTTTG |
| SEQ ID NO: 112 | PCV3-cap-R-1 | ATGAGACACAGAGCTATATTCAGAAG |
| SEQ ID NO: 114 | PCV3-cap-R-2 | GGAGCCAAGTGTTTGTGGT |
| SEQ ID NO: 116 | PCV3-cap-R-3 | GGAACTACCAGCGCTCACC |
| SEQ ID NO: 118 | PCV3-rep-R-1 | TTCCAACCTCTTTGCCGATAATAA |
| SEQ ID NO: 120 | PCV3-rep-R-2 | TGTCTCCATGTCTTTCTTTACCC |
| SEQ ID NO: 122 | PCV3-rep-R-3 | AGGCCAGCGAGTATTGCAAG |
| SEQ ID NO: 124 | PCV3-rep-R-4 | CCCCATTATAACCATCCCACCAAG |
| SEQ ID NO: 126 | CSFV-5′UTR-R-1 | AACTATACGTGCTGTAAGTTACAGAG |
| SEQ ID NO: 128 | CSFV-5′UTR-R-2 | ATGGTACCCCTCTCACCAC |
| SEQ ID NO: 130 | CSFV-5′UTR-R-3 | CCTCACTTTGTGTTTGATGATCAG |
| SEQ ID NO: 132 | CSFV-5′UTR-R-4 | AGCGTTGTCATATTGAGTACCGC |
| SEQ ID NO: 134 | CSFV-E2-R-1 | ACAGGAAGTTTTCCTTACAGGTC |
| SEQ ID NO: 136 | CSFV-E2-R-2 | GGCGAGTATCAGTACTGGTTTGA |
| SEQ ID NO: 138 | CSFV-E2-R-3 | CAACTGAATGACGGGACCGTTA |
| SEQ ID NO: 140 | CSFV-E2-R-4 | GGTGGACGGGTGTCATAGAGT |
| SEQ ID NO: 142 | FMDV-polyprotein-R-1 | TTCTTCTCTGACGTCAGGTCAAAC |
| SEQ ID NO: 144 | FMDV-polyprotein-R-2 | GGAGGGACCTTACGCCG |
| SEQ ID NO: 146 | FMDV-polyprotein-R-3 | CGTCAGGTCCAGAGTGGA |
| SEQ ID NO: 148 | FMDV-polyprotein-R-4 | CTGGGTCACAGAACCAATCA |
| SEQ ID NO: 150 | FMDV-5′UTR-R-1 | CCCGAGGTAACACGCGAC |
| SEQ ID NO: 152 | FMDV-5′UTR-R-2 | CGACCGAATAGGCGGAGTG |
| SEQ ID NO: 154 | FMDV-5′UTR-R-3 | CCATTTCTCATGAAAACGGGC |
| SEQ ID NO: 156 | FMDV-5′UTR-R-4 | AAAACGTGCAACTTGAAATCCCG |
| SEQ ID NO: 158 | swIAV-M-R-1 | GTCTTCTAACCGAGGTCGAAACG |
| SEQ ID NO: 160 | swIAV-M-R-2 | CAGCAGAGTAACTGAGCGC |
| SEQ ID NO: 162 | swIAV-M-R-3 | CCTAGTATGCGCAACCTGTGA |
| SEQ ID NO: 164 | swIAV-M-R-4 | AGCAGCAGAGGCTATGGAAATTG |
| SEQ ID NO: 166 | swIAV-H1-R-1 | ATGAAGGCAATACTAGTAGTCTTGCTAT |
| SEQ ID NO: 168 | swIAV-H1-R-2 | TGTCCAAAATATGTCAAAAGCACAAAAT |
| SEQ ID NO: 170 | swIAV-H1-R-3 | CCACAAATGTGATGACACGTGC |
| SEQ ID NO: 172 | swIAV-H1-R-4 | TCTACAGAAACTTAATATGGCTGGT |
| SEQ ID NO: 174 | swIAV-H3-R-1 | AATTAGATTCCCTGTGCTGTTAATC |
| SEQ ID NO: 176 | swIAV-H3-R-2 | TGATGGAATTTCTCGTTCGTTTTC |
| SEQ ID NO: 178 | swIAV-H3-R-3 | TTGAAGTAACTAATGCTACTGAGCTGG |
| SEQ ID NO: 180 | HPS-vtaA-R-1 | CAGATAACGGTACAACAAATGATGTTTC |
| SEQ ID NO: 182 | HPS-vtaA-R-2 | CCCTGCGCTTGGGTATTTG |
| SEQ ID NO: 184 | HPS-vtaA-R-3 | CGAGCAGGGTCCTAGGG |
| SEQ ID NO: 186 | HPS-vtaA-R-4 | AATGGTGCCAGAATTACCAACGT |
| SEQ ID NO: 188 | HPS-infB-R-1 | ATCCGTTGCTTTCGCACCA |
| SEQ ID NO: 190 | HPS-infB-R-2 | TTCTACACGCTCTAGGTTTGC |
| SEQ ID NO: 192 | HPS-infB-R-3 | TTACCGCACTTAATTCTAATACTTCCG |
| SEQ ID NO: 194 | Pm-plpE-R-1 | ATGAAACAAATCGTTTTAAAAACAAGCT |
| SEQ ID NO: 196 | Pm-plpE-R-2 | AATGGCAAGATTTATGGTGAAATCTACA |
| SEQ ID NO: 198 | Pm-plpE-R-3 | GAATACCGTAACGTTATCTTTATCTACA |
| SEQ ID NO: 200 | Pm-kmt1-R-1 | ATGAATCAAGCGGTCACAGAAAAGA |
| SEQ ID NO: 202 | Pm-kmt1-R-2 | GGCAATAGATCGTGATTTTGGTCG |
| SEQ ID NO: 204 | Pm-kmt1-R-3 | CAGAGAGGTGAAAAATACCCCTAAATAA |
| SEQ ID NO: 206 | Pm-kmt1-R-4 | TGACATTGAATAACTACTTATTGACACA |
| SEQ ID NO: 208 | APP-om1A-R-1 | ATGAATATTGCAACAAAATTAATGGCTA |
| SEQ ID NO: 210 | APP-om1A-R-2 | TTTTTATCTTCTTTTGTGGTTGCCGTAG |
| SEQ ID NO: 212 | APP-om1A-R-3 | TTTCCGCATTACTTTTATCCTTACTT |
| SEQ ID NO: 214 | APP-om1A-R-4 | GCGATGAAAAAGTTAGTTTGAATGAATA |
| SEQ ID NO: 216 | APP-apxIVA-R-1 | CATCTAAATCTAAAGCTAAAGGATCGTA |
| SEQ ID NO: 218 | APP-apxIVA-R-2 | CAAGGATTTTATGAAAACTGGGCGG |
| SEQ ID NO: 220 | APP-apxIVA-R-3 | CCAAGAGGTTCAATACCAAAGTTTT |
| SEQ ID NO: 222 | APP-apxIVA-R-4 | ACTGATATTAATTTATCCGAACTTTGGT |
| SEQ ID NO: 224 | S.suis-gdh-R-1 | ATGTCAAATGCCAAAGCTTACATCC |
| SEQ ID NO: 226 | S.suis-gdh-R-2 | GCCATCGTAGTTCCATACAGAA |
| SEQ ID NO: 228 | S.suis-gdh-R-3 | CTTGTCATGGACTCGTGAAGAAGT |
| SEQ ID NO: 230 | S.suis-gdh-R-4 | AGTAAACCAAACCGTAACCAGT |
| SEQ ID NO: 232 | S.suis-recN-R-1 | ACTGTCGATGATGTTTTGGACTATTTTA |
| SEQ ID NO: 234 | S.suis-recN-R-2 | GAAAACGGGATGACCATCCT |
| SEQ ID NO: 236 | S.suis-recN-R-3 | AGGCCATTGCCCAGAAAAT |
| SEQ ID NO: 238 | S.suis-recN-R-4 | AAAAATGAGCAAGAACACAAGGC |
| SEQ ID NO: 240 | S.suis-gapdh-R-1 | ATGGTAGTTAAAGTTGGTATTAACGGT |
| SEQ ID NO: 242 | S.suis-gapdh-R-2 | AGTTATCGAAGTTGATGGCGAG |
| SEQ ID NO: 244 | S.suis-gapdh-R-3 | CATCCACGCTAACGGTGCTA |
| SEQ ID NO: 246 | S.suis-gapdh-R-4 | CTGCAAACATCGTTCCTAACTCA |
| SEQ ID NO: 248 | E.suis-ppa-R-1 | CTTAAATATGAGTGTGTTGATGGCA |
| SEQ ID NO: 250 | E.suis-ppa-R-2 | CTTTTCAATCTTGTTCTCCAGAATCTA |
| SEQ ID NO: 252 | E.suis-ppa-R-3 | TTGCAAAATTCTTGGTTCTCTAGAAAT |
| SEQ ID NO: 254 | E.suis-g1-R-1 | ATGACAATCCACAAAGTAGCAATCAATG |
| SEQ ID NO: 256 | E.suis-g1-R-2 | GAAGACTCATTATCATATCATGCATACA |
| SEQ ID NO: 258 | E.suis-g1-R-3 | GTTCAGGAAGATTCTTAACTAAGGAAG |
| SEQ ID NO: 260 | E.suis-g1-R-4 | TGCTCCTGTAGTTGTAGGAATAATT |
| SEQ ID NO: 262 | T.gondii-B1-R-1 | CCAGAGTGGATTTCCGTTGGT |
| SEQ ID NO: 264 | T.gondii-B1-R-2 | TCTCTTCACTGTCACGTACGACA |
| SEQ ID NO: 266 | T.gondii-B1-R-3 | GAAGAAGGGTACGTGTTGCATCATA |
| SEQ ID NO: 268 | T.gondii-B1-R-4 | GAGAGACGCTCTACAAAATCCA |
| SEQ ID NO: 270 | T.gondii-RE-R-1 | AGGGAGGAAGACGAAAGTTGT |
| SEQ ID NO: 272 | T.gondii-RE-R-2 | ACAGAAGGGACAGAAGTCGAA |
| SEQ ID NO: 274 | T.gondii-RE-R-3 | TCGTCTCGTCTGGATCGCA |
| SEQ ID NO: 276 | T.gondii-RE-R-4 | GCTGCTTTTCCTGGAGGG |
| SEQ ID NO: 278 | Mhr-p37-R-1 | AATGAAGACAGAAGCGCACATC |
| SEQ ID NO: 280 | Mhr-p37-R-2 | GAAAAGATGGAAGCACTTATAGTAACTA |
| SEQ ID NO: 282 | Mhr-p37-R-3 | ACGTCATCGAAGTTTTTGGTTTTAT |
| SEQ ID NO: 284 | Mhr-16SrRNA-R-1 | GCGATAAATAACTATATCCGGTATTAGC |
| SEQ ID NO: 286 | Mhr-16SrRNA-R-2 | CTCGTGTCGTGAGATGTTAGGTTAAG |
| SEQ ID NO: 288 | Mhr-16SrRNA-R-3 | TTTTTGCTAAGTCTGGAGTTAAATGCTG |
| SEQ ID NO: 290 | Mhp-p36-R-1 | TGCAGCAATGAATCAAGGACT |
| SEQ ID NO: 292 | Mhp-p36-R-2 | ACAGTTCCACTACCGATAACTTTT |
| SEQ ID NO: 294 | Mhp-p36-R-3 | GGTGAACATGGTGATTCATCTTTTGTT |
| SEQ ID NO: 296 | Mhp-p102-R-1 | GCATTTGAACAGCTTTTAATTTCGC |
| SEQ ID NO: 298 | Mhp-p102-R-2 | AAACTTCCTTTCTCTGCAAATGATT |
| SEQ ID NO: 300 | Mhp-p102-R-3 | ACTTAAATAGACAGTTCCGATTTTGGTA |
| SEQ ID NO: 302 | Mhp-p102-R-4 | TATTCTTATCTAAATCAGATCCCGAGTC |

2. Amplicon Ds DNA Library Construction

1). Sample Processing

The sample types mainly include, but are not limited to, porcine lung tissue, whole blood or serum, nasal swab, or pharyngeal swab, and the sample microbial genome was extracted by using, but not limited to, HiPure MicroBiome DNA Kit (Magen Biotechnology Limited, http://www.magentec.com.cn/), TIANamp Virus DNA/RNA Kit (Tiangen Biochemical Technology Co., Ltd, http://www.tiangen.com.cn/), and GoScript™ Reverse Transcription Mix, Random Primer (Promega Corporation, http://www.promega.com.cn/), and the like. The microbial DNA extracted from a single sample was quantified on ds DNA by using a Qubit™ 4 Fluorometer, and the concentration of each sample was controlled to be within 100 ng/μL.

TABLE 3
Sample information

| No. | Sample No. | Sample type | Source |
|---|---|---|---|
| 1 | B1 | Whole blood | Inner Mongolia, China |
| 2 | B2 | Whole blood | Inner Mongolia, China |
| 3 | B3 | Whole blood | Inner Mongolia, China |
| 4 | B4 | Whole blood | Inner Mongolia, China |
| 5 | B5 | Whole blood | Inner Mongolia, China |
| 6 | S1 | Serum | Inner Mongolia, China |
| 7 | S2 | Serum | Inner Mongolia, China |
| 8 | S3 | Serum | Inner Mongolia, China |
| 9 | S4 | Serum | Inner Mongolia, China |
| 10 | S5 | Serum | Inner Mongolia, China |
| 11 | NS1 | Nasal swab | Inner Mongolia, China |
| 12 | NS2 | Nasal swab | Inner Mongolia, China |
| 13 | NS3 | Nasal swab | Inner Mongolia, China |
| 14 | NS4 | Nasal swab | Inner Mongolia, China |
| 15 | NS5 | Nasal swab | Inner Mongolia, China |
| 16 | TS1 | Pharyngeal swab | Inner Mongolia, China |
| 17 | TS2 | Pharyngeal swab | Inner Mongolia, China |
| 18 | TS3 | Pharyngeal swab | Inner Mongolia, China |
| 19 | TS4 | Pharyngeal swab | Inner Mongolia, China |
| 20 | TS5 | Pharyngeal swab | Inner Mongolia, China |
| 21 | L1 | Lung tissue | Inner Mongolia, China |
| 22 | L2 | Lung tissue | Inner Mongolia, China |
| 23 | L3 | Lung tissue | Inner Mongolia, China |
| 24 | L4 | Lung tissue | Inner Mongolia, China |
| 25 | L5 | Lung tissue | Inner Mongolia, China |

2). First Cycle of PCR Amplification

The total DNA of the microorganism of a single sample was taken as a template, first cycle of PCR amplification was performed by using a primer pool of an amplicon sequencing library synthesized by MGI Tech Co., Ltd., and the reaction system was as follows:

TABLE 4
First cycle of PCR reaction system

| Reaction system | | |
|---|---|---|
| PCR Enzyme Mix | 12.5 | μL |
| PCR Clean Enzyme | 0.5 | μL |
| Primer pool | 2 | μL |
| DNA (100 ng) | [100 ng/C (ng/μL)] μL | |
| NF water | (10-DNA) μL | |
| Total | 25 | μL |

The first cycle of PCR reaction solution was vortexed 3 times for 3 s each time, instantaneous centrifugation was performed, the reaction solution was collected to a bottom of a tube, then first cycle of PCR amplification was performed, and the reaction procedures were as follows: heated lid at 105° C.; 37° C. for 5 min, 95° C. for 10 min, 1 cycle; 95° C. for 20 s, 64° C. for 1 min, 60° C. for 1 min, 72° C. for 30 s, 13 cycles; and 12° C. Hold. After the reaction was completed, the reaction solution was centrifuged instantaneously and collected to the bottom of the tube.

3). First Cycle of PCR Product Purification

DNA Clean Beads were taken 30 min in advance and placed at room temperature, the DNA Clean Beads were fully shaken and uniformly mixed before used, 30 μL of the DNA Clean Beads were put in 25 clean 1.5 mL centrifuge tubes according to 25 sample volumes, and sample numbers were marked; all 25 μL products corresponding to the numbers of the first cycle of PCR were transferred to centrifuge tubes, the products were gently pipetted by a pipettor at least 10 times until the products were completely mixed, and it was ensured that all liquid and magnetic beads in a suction head were injected into the tubes for the last time, and incubated at room temperature for 5 min.

After instantaneous centrifugation, a 1.5 mL centrifuge tube was placed on a magnetic rack and left to stand for 2-5 min until the liquid was clear, the liquid was carefully sucked by using a pipettor, the supernatant was discarded; a 1.5 mL centrifuge tube was kept on a magnetic rack, 200 μL of freshly prepared 80% ethanol was added to rinse the magnetic beads and the tube wall, the liquid was left to stand for 30 s and carefully sucked, the supernatant was discarded, and this process was repeated again to ensure the liquid in the tube to be thoroughly sucked for the second time; a 1.5 mL centrifuge tube was kept on a magnetic rack, a tube cover was opened and dried at room temperature until the surface of the magnetic beads had no reflection and no crack; a 1.5 mL centrifuge tube was taken and added with 6.5 μL TE Buffer for DNA elution, and the buffer was gently pipetted by a pipettor until the buffer was completely mixed, then incubated at room temperature for 5 min, and subjected to instantaneous centrifugation. In this step, the magnetic beads were not removed.

6.5 μL of the purified product (containing magnetic beads) was stored in a refrigerator at −20° C.

4). Second Cycle of PCR Amplification

The first cycle of PCR purified product was taken as a template, a customized panel provided by MGI Tech Co., Ltd. was subjected to second cycle of PCR amplification, and the reaction system was as follows:

TABLE 5
Second cycle of PCR reaction system

| Reaction system | | |
|---|---|---|
| PCR Enzyme Mix | 12.5 | μL |
| PCR Clean Enzyme | 0.5 | μL |
| PCR Additive | 0.5 | μL |
| Custom Panel PCR Block | 1 | μL |
| PCR Barcode Primer Mix | 4 | μL |
| DNA | 6.5 | μL |
| Total | 25 | μL |

The prepared second cycle of PCR reaction solution was vortexed 3 times for 3 s each time, instantaneous centrifugation was performed, the reaction solution was collected to a bottom of a tube, then second cycle of PCR amplification was performed, and the reaction procedures were as follows: heated lid at 105° C.; 37° C. for 5 min, 95° C. for 10 min, 1 cycle; 95° C. for 20 s, 64° C. for 1 min, 60° C. for 1 min, 72° C. for 30 s, 27 cycles; and 12° C. Hold. After the reaction was completed, the reaction solution was centrifuged instantaneously and collected to the bottom of the tube.

5). Second Cycle of PCR Product Purification

DNA Clean Beads were taken 30 min in advance and placed at room temperature, the DNA Clean Beads were fully shaken and uniformly mixed, 25 μL of the DNA Clean Beads were put in 25 clean 1.5 mL centrifuge tubes according to 25 sample volumes, and sample numbers were marked; all 25 μL products corresponding to the numbers of the second cycle of PCR in the step 4 were transferred to centrifuge tubes, the products were gently pipetted by a pipettor at least 10 times until the products were completely mixed, and it was ensured that all liquid and magnetic beads in a suction head were injected into the tubes for the last time, and incubated at room temperature for 5 min.

After instantaneous centrifugation, a 1.5 mL centrifuge tube was placed on a magnetic rack and left to stand for 2-5 min until the liquid was clear, the liquid was carefully sucked by using a pipettor, the supernatant was discarded; a 1.5 mL centrifuge tube was kept on a magnetic rack, 200 μL of freshly prepared 80% ethanol was added to rinse the magnetic beads and the tube wall, the liquid was left to stand for 30 s and carefully sucked, the supernatant was discarded, and this process was repeated again to ensure the liquid in the tube to be thoroughly sucked for the second time; a 1.5 mL centrifuge tube was kept on a magnetic rack, a tube cover was opened and dried at room temperature until the surface of the magnetic beads had no reflection and no crack; a 1.5 mL centrifuge tube was taken and added with 25 μL TE Buffer for DNA elution, and the buffer was gently pipetted by a pipettor until the buffer was completely mixed, then incubated at room temperature for 5 min, and subjected to instantaneous centrifugation; the 1.5 mL centrifuge tube was placed on the magnetic rack and left to stand for 2-5 min until the liquid was clear, and 23 μL supernatant was transferred to a new PCR tube. The magnetic beads need to be removed clean in this step.

23 μL of the purified product was stored in a refrigerator at −20° C., at this point, amplicon ds DNA library construction was completed.

3. Amplicon Ss DNA Library Construction

1). Quality Inspection of Ds DNA Library and Construction of Ds DNA Mixed Library

The constructed single ds DNA library was quantified by using a Qubit™ 4 Fluorometer according to an operation instruction, wherein the library required that the yield of a final PCR product was greater than or equal to 5 ng/μL; and then according to the quantitative result of the DNA library, the samples to be sequenced were mixed according to the quality of the barcode numbers and the like, wherein the total amount of the mixed final library was required to be 400 ng, the total volume was less than or equal to 48 μL, and the sampling volume of a single library was ensured to be greater than or equal to 1 μL.

After quantification by a Qubit™ 4 Fluorometer, 25 samples all met the quality requirement, and then 25 samples were mixed by calculation. Sampling was performed, wherein the sampling quantity of a single library was enlarged by 20 times, and finally, 25 amplicon ds DNA libraries were constructed into 1 amplicon ds DNA mixed library.

2). Denaturation of Ds DNA Library to Ss DNA Library

The single ds DNA mixed library obtained in the step 1 was taken as a template, 400 ng of the ds DNA mixed library was placed in a PCR tube, the PCR tube was supplemented with a TE Buffer to a total volume of 48 μL, and a denaturation reaction was performed. The reaction conditions were as follows: heated lid at 105° C., 95° C. for 3 min, and 95° C. Hold. After the reaction was finished, the PCR tube was quickly placed on ice for an ice bath for 2 min, and then ds DNA mixed library in the tube was subjected to instantaneous centrifugation to obtain an ss DNA initial library.

3). Amplicon Ss DNA Library Circularization

The above denatured amplicon ss DNA library was taken as a template, Amplicon library circularization was performed by using MGIEasy circularization kit, and the reaction system was as follows:

TABLE 6
Reaction system for ss DNA amplicon library circularization

| Reaction system | | |
|---|---|---|
| Splint Buffer | 11.5 | μL |
| DNA Rapid Ligase | 0.5 | μL |
| ss DNA | 48 | μL |
| Total | 60 | μL |

The prepared PCR reaction solution was vortexed 3 times for 3 s each time, instantaneous centrifugation was performed, the reaction solution was collected to a bottom of a tube, then PCR amplification was performed, and the reaction procedures were as follows: heated lid at 105° C.; 37° C. for 30 min, and 4° C. Hold. After the reaction was completed, the PCR tube was instantaneously centrifuged and placed on ice to immediately proceed to the reaction in step 4.

4). Ss DNA Library Enzyme Digestion

The circularized amplicon ss DNA library was taken as a template, the reaction in step 3 was followed by the reaction in step 4, and the reaction system was as follows:

TABLE 7
Reaction system for ss DNA library enzyme digestion

| Reaction system | | |
|---|---|---|
| Digestion Buffer | 1.4 | μL |
| Digestion Enzyme | 2.6 | μL |
| ss DNA | 60 | μL |
| Total | 66 | μL |

The prepared enzyme digestion solution was vortexed 3 times for 3 s each time, instantaneous centrifugation was performed, the reaction solution was collected to a bottom of a tube, then PCR amplification was performed, and the reaction procedures were as follows: heated lid at 105° C.; 37° C. for 30 min, and 4° C. Hold. After the reaction was completed, the reaction solution was centrifuged instantaneously and collected to the bottom of the tube. Then 7.5 μL of Digestion Stop Buffer was immediately added to the PCR tube, vortexed 3 times for 3 s each time, the reaction solution was collected to the bottom of the tube after instantaneous centrifugation, and the whole reaction solution was sucked and transferred to a new 1.5 mL centrifuge tube.

5). Purification of Enzyme Digestion Products

The DNA Clean Beads were taken out in advance during the enzyme digestion reaction and placed at room temperature, and the DNA Clean Beads was fully shaken and mixed uniformly before use; and 170 μL of the mixture was put into the 1.5 mL centrifuge tube filled with the reaction solution and gently pipetted by a pipettor at least 10 times until the mixture was completely mixed, and it was ensured that all liquid and magnetic beads in a suction head were injected into the tubes for the last time, and incubated at room temperature for 10 min.

After instantaneous centrifugation, a 1.5 mL centrifuge tube was placed on a magnetic rack and left to stand for 2-5 min until the liquid was clear, the liquid was carefully sucked by using a pipettor, the supernatant was discarded; a 1.5 mL centrifuge tube was kept on a magnetic rack, 500 μL of freshly prepared 80% ethanol was added to rinse the magnetic beads and the tube wall, the liquid was left to stand for 30 s and carefully sucked, the supernatant was discarded, and this process was repeated again to ensure the liquid in the tube to be thoroughly sucked for the second time; a 1.5 mL centrifuge tube was kept on a magnetic rack, a tube cover was opened and dried at room temperature until the surface of the magnetic beads had no reflection and no crack; a 1.5 mL centrifuge tube was taken and added with 22 μL TE Buffer for DNA elution, and the buffer was gently pipetted by a pipettor until the buffer was completely mixed, then incubated at room temperature for 10 min, and subjected to instantaneous centrifugation; the 1.5 mL centrifuge tube was placed on the magnetic rack and left to stand for 2-5 min until the liquid was clear, and 20 μL supernatant was transferred to a new PCR tube. The magnetic beads need to be removed clean in this step.

20 μL of the purified product was stored in a refrigerator at −20° C., at this point, amplicon ss DNA library was successfully constructed.

4. Amplicon DNB Library Construction

1). Amplicon Ss DNA Equilibrium Library Construction

The conversion formula of the concentration is as follows: C(fmol/μL)=3030*C(ng/μL)/N.

According to the quantitative result of the Qubit™ 4 Fluorometer, the concentration of the amplicon ss DNA library constructed in this embodiment was greater than or equal to 2 fmol/μL, and the quality control requirement was met. After ss DNA quantification was performed on standard products provided in the Qubit® ssDNA Assay Kit, the standard products were calculated using the following two formulas:

ss DNA input (μL)=44/C(fmol/μL); (1)

the standard ss DNA input (μL)=16/C(fmol/μL). (2)

The ss DNA input and the standard ss DNA input were mixed to construct an amplicon ss DNA equilibrium library.

2). Amplicon DNB Library Construction

The amplicon ss DNA equilibrium library in the step 1 was taken as a template, DNB preparation was performed by using an MGISEQ-200RS high-throughput sequencing kit (FCS PE 100), and two cycles of reactions were performed in total, wherein the first cycle of reaction was PCR primer hybridization, and the reaction system was as follows:

TABLE 8
DNB preparation reaction system 1

| Reaction system | |
|---|---|
| ss DNA equilibrium library | V μL |
| TE Buffer | 20-V μL |
| DNB preparation buffer | 20 μL |
| Total | 40 μL |

The prepared DNB reaction mixed solution 1 was vortexed, uniform mixed, centrifuged for 5 s by using a mini centrifuge, and then placed in a PCR instrument for primer hybridization, wherein the reaction conditions were as follows: heated lid at 105° C., 95° C. for 1 min, 65° C. for 1 min, 40° C. for 1 min, and 4° C. Hold. After the reaction was completed, the solution was subjected to a second cycle of reaction, namely, a DNB rolling circle amplification reaction. Firstly the PCR reaction tube was taken out, centrifuged by a mini-centrifuge for 5 s, and then placed on ice to prepare a second cycle of reaction solution by DNB, wherein the second cycle of reaction system was as follows:

TABLE 9
DNB preparation reaction system 2

| Reaction system | | |
|---|---|---|
| DNB reaction mixed solution 1 | 40 | μL |
| DNB polymerase mixed solution I | 40 | μL |
| DNB polymerase mixed solution II (LC) | 4 | μL |
| Total | 84 | μL |

The prepared DNB mixed reaction solution 2 was vortexed by using a vortex oscillator and uniformly mixed, centrifuged for 5 s by using a mini centrifuge, and immediately placed in a PCR instrument for DNB rolling circle amplification, wherein the reaction conditions were as follows: heated lid at 35° C., 30° C. for 25 min, 4° C. Hold. After the reaction was completed, the solution was immediately added with 20 μL of DNB termination buffer solution, and the mixture was slowly pipetted for 5 to 8 times to be well mixed by using a wide-mouthed pipette tip without vortexing or violently pipetting.

3). DNB Quality Inspection

After DNB preparation was completed, 2 μL of DNB mixed reaction solution was subjected to concentration detection by using Qubit® ssDNA Assay Kit and Qubit® Fluorometer instruments, and the concentration of the amplicon DNB=15.5 ng/μL, which met the quality control requirement. DNB was stored at 4° C. for future use and prepared for on-line sequencing.

5. On-Line Sequencing

The prepared DNB was prepared for on-line sequencing according to the instructions of the high-throughput (rapid) sequencing reagent set in MGI Tech, which was the MGISEQ-200RS high-throughput rapid sequencing reagent set (FCS PE100).

The sequencing data were extracted for analysis and comparison, a total summary report was generated after sequencing was completed, the summary report was opened by a webpage and viewed, the summary report shows that the sequencing quality is high, the data result of greater than or equal to Q30 is more than 90%, the Barcode can be normally identified, all samples can be correctly distinguished, and the capture rate of a pathogenic target gene area has certain difference according to the difference of pathogen load in the samples. The sequencing result is accurate and reliable, and the designed amplification primer has high efficiency and good specificity.

According to the sequencing results of the target gene, after data homogenization and calculation of the pathogen load, the detection of the pathogen is judged according to the calculation result of the pathogenic target gene. It has been proved by practice that the pathogenic genes in the panel are detected in the above 25 samples, thus successfully implementing the rapid, accurate, and simultaneous diagnosis of 15 pathogens in the porcine respiratory tract.

Therefore, the method for simultaneous detection of 15 porcine pathogens through high-throughput targeted amplicon sequencing provided by the present invention is successfully established.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

1. A primer group for a simultaneous detection of 15 porcine pathogens through high-throughput targeted amplicon sequencing, wherein the primer group is configured to amplify pathogenic target genes, the pathogenic target genes are as follows:

| Pathogen name | Target gene | Sequence No. |
|---|---|---|
| African swine fever | ASFV-p72 | SEQ ID NO: 303 |
| virus | ASFV-p54 | SEQ ID NO: 304 |
| | ASFV-EP402R | SEQ ID NO: 305 |
| | ASFV-MGF505-1R | SEQ ID NO: 306 |
| | ASFV-MGF505-2R | SEQ ID NO: 307 |
| | ASFV-MGF360-13L | SEQ ID NO: 308 |
| | ASFV-MGF360-14L | SEQ ID NO: 309 |
| | ASFV-K205R | SEQ ID NO: 310 |
| Porcine reproductive | PRRSV-NA-orf6/M | SEQ ID NO: 311 |
| and respiratory | PRRSV-NA-orf7/N | SEQ ID NO: 312 |
| syndrome virus | PRRSV-EU-orf6/M | SEQ ID NO: 313 |
| | PRRSV-EU-orf7/N | SEQ ID NO: 314 |
| Pseudorabies virus | PRV-gE | SEQ ID NO: 315 |
| | PRV-gB | SEQ ID NO: 316 |
| Porcine circovirus | PCV2-cap | SEQ ID NO: 317 |
| | PCV2-rep | SEQ ID NO: 318 |
| | PCV3-cap | SEQ ID NO: 319 |
| | PCV3-rep | SEQ ID NO: 320 |
| Classical swine fever | CSFV-5′UTR | SEQ ID NO: 321 |
| virus | CSFV-E2 | SEQ ID NO: 322 |
| Foot and mouth | FMDV-3D-1C2B(polyprotein) | SEQ ID NO: 323 |
| diseases virus | FMDV-5′UTR | SEQ ID NO: 324 |
| Swine influenza A | swIAV-M | SEQ ID NO: 325 |
| virus | swIAV-H1 | SEQ ID NO: 326 |
| | swIAV-H3 | SEQ ID NO: 327 |
| Haemophilus parasuis | HPS-vtaA | SEQ ID NO: 328 |
| | HPS-infB | SEQ ID NO: 329 |
| Pasteurella multocida | Pm-plpE | SEQ ID NO: 330 |
| | Pm-kmt1 | SEQ ID NO: 331 |
| Actinobacillus | APP-omlA | SEQ ID NO: 332 |
| pleuropneumoniae | APP-apxIVA | SEQ ID NO: 333 |
| Streptococcus suis | S.suis-gdh | SEQ ID NO: 334 |
| | S.suis-recN | SEQ ID NO: 335 |
| | S.suis-gapdh | SEQ ID NO: 336 |
| Eperythrozoon suis | E.suis-ppa | SEQ ID NO: 337 |
| | E.suis-g1 | SEQ ID NO: 338 |
| Toxoplasma gondii | T.gondii-B1 | SEQ ID NO: 339 |
| | T.gondii-RE | SEQ ID NO: 340 |
| Mycoplasma hyorhinis | Mhr-p37 | SEQ ID NO: 341 |
| | Mhr-16SrRNA | SEQ ID NO: 342 |
| Mycoplasma | Mhp-p36 | SEQ ID NO: 343 |
| hyopneumoniae | Mhp-p102 | SEQ ID NO: 344; |

nucleotide sequences of the pathogenic target genes are set forth in SEQ ID NO: 303 to SEQ ID NO: 344;
the primer group comprises a first primer group and a second primer group, wherein the first primer group comprises forward primer sequences set forth in singular sequence numbers in SEQ ID NO: 1 to SEQ ID NO: 302, an adapter sequence set forth in SEQ ID NO: 345 is added at a 5′-end of each of forward primers, the second primer group comprises reverse primer sequences set forth in double sequence numbers in SEQ ID NO: 1 to SEQ ID NO: 302, and an adapter sequence set forth in SEQ ID NO: 346 is added at a 5′-end of each of reverse primers.

2. A multiplex PCR reagent for a simultaneous detection of 15 porcine pathogens through high-throughput targeted amplicon sequencing, comprising a first PCR reagent and a second PCR reagent, wherein the first PCR reagent comprises the first primer group of the primer group according to claim 1, and the second PCR reagent comprises the second primer group of the primer group according to claim 1.

3. The multiplex PCR reagent according to claim 2, wherein each primer in the first primer group has a concentration of 10 nmol/μL in the first PCR reagent, and each primer in the second primer group has a concentration of 10 nmol/μL in the second PCR reagent.

4. A kit for a simultaneous detection of 15 porcine pathogens through high-throughput targeted amplicon sequencing, comprising the primer group according to claim 1.

5. Use of the primer group according to claim 1 in constructing a high-throughput amplicon sequencing library for a simultaneous detection of 15 porcine respiratory pathogens.

6. Use of the primer group according to claim 1 in high-throughput amplicon sequencing for a simultaneous detection of 15 porcine respiratory pathogens in non-diagnostic and therapeutic purposes.

7. Use of the primer group according to claim 1 in detecting 15 porcine respiratory pathogenic genes in a test sample for non-diagnostic and therapeutic purposes.

8. A kit for a simultaneous detection of 15 porcine pathogens through high-throughput targeted amplicon sequencing, comprising the multiplex PCR reagent according to claim 2.

9. The kit for the simultaneous detection of the 15 porcine pathogens through the high-throughput targeted amplicon sequencing according to claim 8, wherein in the multiplex PCR reagent, each primer in the first primer group has a concentration of 10 nmol/μL in the first PCR reagent, and each primer in the second primer group has a concentration of 10 nmol/μL in the second PCR reagent.

10. Use of the multiplex PCR reagent according to claim 2 in constructing a high-throughput amplicon sequencing library for a simultaneous detection of 15 porcine respiratory pathogens.

11. The use according to claim 10, wherein in the multiplex PCR reagent, each primer in the first primer group has a concentration of 10 nmol/μL in the first PCR reagent, and each primer in the second primer group has a concentration of 10 nmol/μL in the second PCR reagent.

12. Use of the kit according to claim 4 in constructing a high-throughput amplicon sequencing library for a simultaneous detection of 15 porcine respiratory pathogens.

13. Use of the multiplex PCR reagent according to claim 2 in high-throughput amplicon sequencing for a simultaneous detection of 15 porcine respiratory pathogens in non-diagnostic and therapeutic purposes.

14. The use according to claim 13, wherein in the multiplex PCR reagent, each primer in the first primer group has a concentration of 10 nmol/μL in the first PCR reagent, and each primer in the second primer group has a concentration of 10 nmol/μL in the second PCR reagent.

15. Use of the kit according to claim 4 in high-throughput amplicon sequencing for a simultaneous detection of 15 porcine respiratory pathogens in non-diagnostic and therapeutic purposes.

16. Use of the multiplex PCR reagent according to claim 2 in detecting 15 porcine respiratory pathogenic genes in a test sample for non-diagnostic and therapeutic purposes.

17. The use according to claim 16, wherein in the multiplex PCR reagent, each primer in the first primer group has a concentration of 10 nmol/μL in the first PCR reagent, and each primer in the second primer group has a concentration of 10 nmol/μL in the second PCR reagent.

18. Use of the kit according to claim 4 in detecting 15 porcine respiratory pathogenic genes in a test sample for non-diagnostic and therapeutic purposes.
